(19) United States
(12) Patent Application Publication
(10) Pub. No.: US 2026/0357285 A1
(43) Pub. Date: Nov. 12, 2026

(54) Flavonoid-Dependent Fertility

(71) Applicant: Syngenta Crop Protection AG, Basel (CH)

(72) Inventors: Rachel Egger, RTP, NC (US); Jared Carter, RTP, NC (US); Julie Leonard Green, RTP, NC (US)

(73) Assignee: Syngenta Crop Protection AG, Basel (CH)

(21) Appl. No.: 18/997,559

(22) PCT Filed: Jul. 26, 2023
(86) PCT No.: PCT/US2023/071016

§ 371 Date: Jan. 22, 2025

Related U.S. Application Data
(60) Provisional application No. 63/369,975, filed on Aug. 1, 2022.

(51) Int. Cl. C12N 15/82; A01N 25/12; A01N 43/16; A01P 21/00

(52) U.S. Cl. CPC C12N 15/8287; A01N 25/12; A01N 43/16; A01P 21/00

(57) ABSTRACT

Flavonoid-dependent fertility provides a safe and convenient method to control fertilization between plants. Shown previously in maize and petunia by mutations to chalcone synthase, quercetin, kaempferol, and other flavonoids restore fertility when applied to the pollen or flowers. Here, novel gene targets, and mutations thereto, have been discovered in wheat, rice, and maize. By mutating these novel genes in each of the three genomes of wheat, we provide for flavonoid-dependent fertility in wheat. Also provided are a rice FDF gene and an improved FDF system in maize.

### RELATED APPLICATION INFORMATION

This application claims priority under 35 U.S.C. § 119 to U.S. Application No. 63/369,975, filed Aug. 1, 2022, the contents of which are incorporated herein by reference in their entirety.

### FIELD OF THE INVENTION

This invention relates to methods for the regulation of fertility in plants, and more particularly to the creation of flavonoid-dependent fertility in wheat and maize plants by mutations in novel genes, and to the restoration of fertility in these plants by providing fertility restoring flavonoids at the plant pollen sites.

### SEQUENCE LISTING

This application is accompanied by a sequence listing entitled 82692PCT.xml, created Jul. 24, 2023, which is approximately 200 kilobytes in size. This sequence listing is incorporated herein by reference in its entirety. This sequence listing is submitted herewith and complies with 37 C.F.R. §§ 1.831-1.835.

### BACKGROUND

Control of male fertility in crop plants is a key factor required to maintain seed purity and control cost of goods in hybrid seed production. In maize, control of male fertility is frequently accomplished through removal of male reproductive tissues (detasseling). In both cereals and maize, male fertility can also be controlled using Cytoplasmic Male Sterility (CMS). Detasseling is challenged by high costs in hybrid seed production, while CMS systems are challenged by high cost of goods in CMS line increase, added complexity in breeding, and unstable male fertility in hybrids. A class of alternative hybrid seed production technologies that are designed to overcome these challenges associated with detasseling and CMS are termed “conditional male sterility”. In plants bred for conditional male sterility, growers can control whether pollen is viable or sterile through either selection of specific growing conditions or exogenous application of chemistries that impact pollen viability.

One form of conditional male sterility in maize and wheat is Flavonoid-Dependent Fertility (FDF). Flavonoids are a class of secondary metabolites in plants that fulfill diverse biological roles including tissue pigmentation, acting as signaling compounds, and mediating reactive oxygen species (ROS) homeostasis. Flavonoids are believed essential to reproduction in maize and wheat due to their role in mediating ROS homeostasis during pollen tube germination that controls pollen tube bursting. These essential flavonoids are accumulated in the pollen grains prior to pollen maturity so that the compounds are immediately available when the pollen is shed and lands on a receptive stigma. Pollen tube bursting is normally a tightly regulated process that maintains pollen tube structural integrity during growth through a receptive stigma. When pollen tubes reach the synergids, pollen tube bursting is initiated to release sperm cells and allow for the double fertilization necessary as part of plant sexual reproduction. For maize and wheat plants in which the pollen grains are deficient in these essential flavonoids, pollen tube growth fails prematurely. Pollen tubes either never successfully grow into the stigma or the tubes burst prematurely before reaching the synergids. This failed pollen tube growth completely prevents fertilization, thus maize and wheat species plants in which the pollen is deficient in flavonoids are male-sterile.

Use of flavonols to restore fertility has been previously observed in maize (see, e.g., E. H. Coe, et al., White pollen in maize, J. HEREDITY 72:318-320 (1981)). An early example was the result of random mutations impacting two copies of chalcone synthase. One copy of chalcone synthase (WHITE POLLEN 1, WHP1) on maize chromosome 2 is expressed in anthers during late stages of pollen development. A plant homozygous for the whp1 mutant allele will produce and shed pollen that is less yellow in color (colloquially called “white pollen”) than typical pollen. If this white pollen is applied to a standard wild-type maize silk, normal pollen tube growth and fertilization can occur. The second chalcone synthase, (COLORLESS2, C2), on maize chromosome 4 is expressed in numerous tissue types, including silks. If mutations to both whp1 and c2 are homozygous in the same plant, the plant will produce and shed pollen that is less yellow in color and is incapable of growing a normal pollen tube during self-pollination or fertilizing another maize plant through cross-pollination. It was later discovered that this white pollen is conditionally sterile and can be restored to fully fertile through the addition of exogenous flavonoids at the point of pollen and silk interaction (Y. Mo, et al., Biochemical complementation of chalcone synthase mutants defines a role for flavonols in functional pollen, PROC. NAT'L ACAD. SCI. USA 89:7213-7217 (1992)). This exogenous flavonoid application can be accomplished by coating the mature white pollen grains with dry powdered flavonoids or by applying flavonoids directly to silks prior to applying the white pollen.

Discovery of the c2/whp1 conditional male sterility system prompted patent applications seeking to commercialize the technology in maize through multiple approaches, including use of GM technology and inducible promoters (potential reference to U.S. Pat. Nos. 5,432,068 and 5,733,759). While theoretically a promising alternative to detasseling or CMS, the technology was not commercialized for maize seed production. The most critical shortcoming is the mutation to c2 that impacts both male and female reproductive tissues other than pollen. Loss of this key chalcone synthase inhibits the downstream synthesis of flavonoids and anthocyanins in silks, anthers, kernel aleurone, and other tissues. This loss of downstream metabolites potentially disrupts ROS signaling, inhibits response to abiotic stress in tissues that impact female receptivity and yield, and may alter plant visual phenotype in ways that are unappealing to growers. In addition, the c2/whp1 system was not easily translated into crops other than maize.

### SUMMARY

Here we report the discovery of a novel FDF system in maize and wheat based on mutations to anther-specific chalcone synthase genes. This novel system, termed Flavonoid Dependent Fertility (“FDF”) demonstrates complete male sterility that can be restored to male fertility through application of exogenous flavonoids, but without the critical shortcomings of c2/whp1. FDF mutant alleles do not impact downstream synthesis of flavonoids and anthocyanins in other tissues beyond the anther tapetal cell layer and therefore cannot impact the color or stress response capacity of female reproductive tissues.

Within the scope of the invention is a method of seed production, comprising: (a) obtaining a first plant which is a flavonoid-dependent fertile (“FDF”) plant with a mutation in an FDF gene; (b) obtaining a second plant, which is male fertile; (c) pollinating the FDF plant with pollen from the second plant; and (d) obtaining progeny seed. An FDF gene may be TaFDF1A, TaFDF1B, TaFDF1D, or a combination thereof (SEQ ID NOs: 1-3), or homologues thereof; or ZmFDF1, or ZmFDF2, or a combination thereof (SEQ ID NOs 4-5), or homologues thereof, such as OsFDF in rice. In one aspect, the first plant and the second plant are monocot plants. In an aspect, the monocot plants are selected from the group consisting of wheat, maize, and rice.

Also within the scope of the invention is a flavonoid-dependent fertile (“FDF”) plant comprising a mutation in an FDF gene. The mutation in the FDF gene may be a knock-out mutation, and it may be homozygous for the knock-out mutation in the FDF gene. FDF gene may be TaFDF1A, TaFDF1B, TaFDF1D, or a combination thereof (SEQ ID NOs: 1-3), or homologues thereof; or ZmFDF1, or ZmFDF2, or a combination thereof (SEQ ID NOs 4-5), or homologues thereof, such as OsFDF in rice. Additionally, the FDF plant further comprises a mutation in C2/WHP or homologues thereof. In one embodiment, the plant is a wheat plant. In an alternative embodiment, the plant is a maize plant.

Another embodiment of the invention is a method of propagating an FDF plant, comprising: (a) obtaining at least one plant, wherein the plant is a flavonoid-dependent fertile (“FDF”) plant comprising a mutation in an FDF gene; (b) applying a composition comprising a flavonoid to the plants of step (a); (c) allowing self-pollination to occur; and (d) obtaining progeny seed thereof.

The flavonoid may be quercetin; the quercetin may be a liquid solution (optionally mixed in water, propylene glycol, or other solvent or solution). The quercetin may be mixed in a solvent or solution at a concentration of 1 mg/L to 100 mg/L, 2.5 mg/L to 50 mg/L, or approximately 5 mg/L; alternatively, the liquid solution is saturated with quercetin. Alternatively, the composition comprising quercetin is a powder which may be a mixture of quercetin and a carrier compound. The mixture of quercetin and carrier compound may be at a ratio between 1000:1 to 1:1000. The carrier compound may be crystalline silica, talc, metallic powder, or mica minerals. In either the liquid formulation or the powder formulation, the flavonoid can be applied to the female organs of a flower of a female parent plant. The flavonoid composition can be applied topically by painting, misting, spraying, and drenching.

Another embodiment of the invention is a method of hybrid seed production, comprising: (a) obtaining at least one inbred female FDF plant, wherein the inbred female FDF plant comprises a mutation in an FDF gene; (b) obtaining pollen from at least one inbred male plant, wherein the inbred male plant produces fertile pollen; (c) pollinating the inbred female FDF plant with the fertile pollen from the inbred male plant; and (d) obtaining hybrid progeny thereof. Another embodiment of the method is a plant made by the previous methods.

Another embodiment of the invention is a method restoring fertility to an FDF plant by applying a composition comprising a flavonoid to the FDF plant. The FDF plant can be maize, wheat, or rice. If wheat, the FDF wheat plant comprises a mutation in a gene selected from the group consisting of SEQ ID NOs: 1-3, and a combination thereof. If maize, the FDF maize plant comprises a mutation in a gene selected from the group consisting of SEQ ID NOs: 4-5 and a combination thereof and optionally further comprising a mutation in C2 (SEQ ID NO: 26) and/or WHP (SEQ ID NO: 28). If the FDF plant is rice, the FDF rice plant comprises a mutation in SEQ ID NO: 57. The flavonoid can comprise quercetin, which can be mixed in liquids such as water, propylene glycol, or other solvents or solutions. Alternatively, the flavonoid can be mixed as a powder with a carrier compound, such as crystalline silica, talc, metallic powder, and mica minerals. This composition can be applied to plant parts such as anthers, silks, stigmas, florets, spikes, leaf whorls, leaf canopy, and/or roots by means of painting, misting, spraying, and root drenching

### BRIEF DESCRIPTION OF THE SEQUENCES IN THE SEQUENCE LISTING

SEQ ID NO: 1 is TraesCS1A02G160300, a gene on Triticum aestivum chromosome 1A (also referred to as TaFDF1A). This gene encodes a functional copy of chalcone synthase that is expressed in the developing anther. Loss of function of this gene is required for FDF in wheat.

SEQ ID NO: 2 is TraesCS1B02G176300, a gene on Triticum aestivum chromosome 1B (also referred to as TaFDF1B). This gene encodes a functional copy of chalcone synthase that is expressed in the developing anther. Loss of function of this gene is required for FDF in wheat.

SEQ ID NO: 3 is TraesCS1D02G157500, a gene on Triticum aestivum chromosome 1D (also referred to as TaFDF1D). This gene encodes a functional copy of chalcone synthase that is expressed in the developing anther. Loss of function of this gene is required for FDF in wheat.

SEQ ID NO: 4 is Zm00001d032662, a gene on Zea mays chromosome 1 (also referred to as ZmFDF1, ZmCHS_Chr1, chalcone synthase2 (chls2), or GRMZM2G380650). The sequence was downloaded from maizeGDB.org and refers to the Zm-B73-REFERENCE-GRAMENE-4.0 genome version. This gene encodes a functional copy of chalcone synthase that is primarily expressed in the meiotic tassel and developing anther with low or zero expression in other tissues. Loss of function of this gene is likely required for FDF in corn.

SEQ ID NO: 5 is Zm00001d013991, a gene on Zea mays chromosome 5 (also referred to as ZmFDF2, ZmCHS_Chr5, chalcone synthase11 (chls11), or GRMZM2G477683). The sequence was downloaded from maizeGDB.org and refers to the Zm-B73-REFERENCE-GRAMENE-4.0 genome version. This gene encodes a functional copy of chalcone synthase that is primarily expressed in the meiotic tassel and developing anther with low or zero expression in other tissues. Loss of function of this gene is likely required for FDF in corn.

SEQ ID NO: 6 is edited TraesCS1A02G160300. It comprises a single base pair deletion relative to SEQ ID NO: 1.

SEQ ID NO: 7 is edited TraesCS1B02G176300. It comprises a two-base pair deletion relative to SEQ ID NO: 2.

SEQ ID NO: 8 is edited TraesCS1D02G157500. It comprises a single base pair deletion relative to SEQ ID NO: 3.

SEQ ID NO: 9 is the DNA sequence encoding the guide RNA sequence targeting SEQ ID NO: 1.

SEQ ID NO: 10 is the DNA sequence encoding the guide RNA sequence targeting SEQ ID NO: 2. We did not find evidence of genome edits with this guide.

SEQ ID NO: 11 is a repeat of SEQ ID NO: 10.

SEQ ID NO: 12 is construct 25206.

SEQ ID NO: 13 is TraesCS2A03G12234, a wheat chromosome A orthologue of maize c2/whp1.

SEQ ID NO: 14 is TraesCS2B03G140060, a wheat chromosome B orthologue of maize c2/whp1.

SEQ ID NO: 15 is TraesCS2D03G11827, a wheat chromosome D orthologue of maize c2/whp1.

SEQ ID NO: 16 is construct 27726.

SEQ ID NO: 17 is construct 27738.

SEQ ID NO: 18 is construct 27769.

SEQ ID NOs: 19-24 are primers LZ311, LZ312, LZ313, LZ314, LZ315, and LZ316, respectively; see Table 5.

SEQ ID NO: 25 is GRMZM2G380650; this is a repeat of SEQ ID NO: 4.

SEQ ID NO: 26 is the reference sequence for C2 in maize.

SEQ ID NO: 27 is the reference sequence for c2 in maize.

SEQ ID NO: 28 is the reference sequence for Whp1 in maize.

SEQ ID NO: 29 is the DNA sequence encoding the guide RNA sequence named ZmFDF1\target37.

SEQ ID NO: 30 is the DNA sequence encoding the guide RNA sequence named ZmFDF1\target74.

SEQ ID NO: 31 is the DNA sequence encoding the guide RNA sequence named ZmFDF2\target72.

SEQ ID NO: 32 is the DNA sequence encoding the guide RNA sequence named ZmFDF2\target74.

SEQ ID NO: 33 is the DNA sequence encoding the guide RNA sequence named ZmFDF2\target2.

SEQ ID NO: 34 is the DNA sequence encoding the guide RNA sequence named ZmFDF2\target16.

SEQ ID NOs: 35-52 are primers and probes listed in Table 9.

SEQ ID NO: 53 is the nucleotide sequence for Os05g0212900, which is presented in reverse orientation (3′-5′).

SEQ ID NO: 54 is the nucleotide sequence for Os07g0214900, which is presented in reverse orientation (3′-5′).

SEQ ID NO: 55 is the nucleotide sequence for Os07g0501100, which is presented in reverse orientation (3′-5′).

SEQ ID NO: 56 is the nucleotide sequence for Os11g0530600, which is presented in reverse orientation (3′-5′).

SEQ ID NO: 57 is the nucleotide sequence for OsFDF (also referred to as Os10g0484800), which is presented in 5′-3′ orientation.

### DEFINITIONS

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

The terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

The term “about” as used herein refers to the usual error range for the respective value readily known to the skilled person in this technical field, for example ±20%, ±10%, or ±5%, are within the intended meaning of the recited value.

The term “allele(s)” means any of one or more alternative forms of a gene, all of which alleles relate to at least one trait or characteristic. In a diploid cell, the two alleles of a given gene occupy corresponding loci on a pair of homologous chromosomes. In some instances (e.g., for QTLs) it is more accurate to refer to “haplotype” (i.e., an allele of a chromosomal segment) instead of “allele”, however, in those instances, the term “allele” should be understood to comprise the term “haplotype”. If two individuals (e.g., two plants) possess the same allele at a particular locus, the alleles are termed “identical by descent” if the alleles were inherited from one common ancestor (i.e., the alleles are copies of the same parental allele). The alternative is that the alleles are “identical by state” (i.e., the alleles appear to be the same but are derived from two different copies of the allele). Identity by descent information is useful for linkage studies; both identity by descent and identity by state information can be used in association studies, although identity by descent information can be particularly useful.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

“Anther specific gene” is defined as a gene with high expression in developing anthers, including in the developing microspores, and with low or zero expression in all other tissues tested including female reproductive tissues.

The term “backcrossing” is understood within the scope of the present disclosure to refer to a process in which a hybrid progeny is repeatedly crossed back to one of the parents.

“Carrier,” as used herein, means a compound, preferably in powdered form, which acts as an agent to accompany collected pollen. Suitable carrier compounds can be, but are not limited to, talc powder, silica powder, and the like.

As used herein, the term “comprising” or “comprise” is open-ended. When used in connection with a subject nucleic acid (or amino acid sequence), it refers to a nucleic acid sequence (or an amino acid sequence) that includes the subject sequence as a part or as its entire sequence.

As used herein, the term “elite line” or “inbred line” refers to any line that has resulted from breeding and selection for superior agronomic performance. An elite line has stable genetics, i.e., it is reasonably or nearly isogenic across its genome. Said another way, an elite line is reasonably or nearly homozygous for all alleles in its genome.

The term “FDF gene” (and also “FDF allele,” which is used interchangeably throughout) refers to a gene that, when mutated or otherwise made defective or inhibited, renders a plant infertile due to a flavonoid deficiency; further, said deficiency is remediable by the application of a flavonol and/or flavonoid. For example, wheat FDF genes include TraesCS1A02G160300 (SEQ ID NO: 1; also referred to as TaFDF1A), TraesCS1B02G176300 (SEQ ID NO: 2; also referred to as TaFDF1B), and TraesCS1D02G157500 (SEQ ID NO: 3; also referred to as TaFDF1D). For example, maize FDF genes include Zm00001d032662 (SEQ ID NO: 4; also referred to as ZmFDF1 or ZmCHS_Chr1) and Zm00001d013991 (SEQ ID NO: 5; also referred to as ZmFDF2 or ZmCHS_Chr5).

The term “gene” refers to a hereditary unit including a sequence of DNA that occupies a specific location on a chromosome and that contains the genetic instruction for a particular characteristic or train in an organism.

“Gene editing” generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e. to be introduced into the plant genome).

Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as without limitation Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540. Alternatively, the Cas12a protein may be from Moraxella bovoculi AAX08_00205 [Mb2Cas12a] or Moraxella bovoculi AAX11_00205 [Mb3Cas12a]. See WO 2017/189308. In certain embodiments, the Cas protein is (modified) C2c2, preferably Leptotrichia wadei C2c2 (LwC2c2) or Listeria newyorkensis FSL M6-0635 °C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

The term “genotype” and variants thereof refers to the genetic composition of an organism, including, for example, whether a diploid organism is heterozygous (i.e., has two different alleles for a given gene or QTL) or homozygous (i.e., has the same allele for a given gene or QTL) for one or more genes or loci (e.g., a SNP, a haplotype, a gene mutation, an insertion, or a deletion). As used herein, the term “at least heterozygous” for a particular allele indicates that at least one copy of the allele is present. For example, a maize plant that is at least heterozygous for a HI allele of a gene has either one or two copies (i.e., is either heterozygous or homozygous) of the HI allele.

The term “germplasm” refers to the totality of the genotypes of a population or other group of individuals (e.g., a species or plant line). The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

The term “haplotype” can refer to the set of alleles an individual inherited from one parent. A diploid individual thus has two haplotypes. The term “haplotype” can be used in a more limited sense to refer to physically linked and/or unlinked genetic markers (e.g., sequence polymorphisms) associated with a phenotypic trait. The phrase “haplotype block” (sometimes also referred to in the literature simply as a haplotype) refers to a group of two or more genetic markers that are physically linked on a single chromosome (or a portion thereof). Typically, each block has a few common haplotypes, and a subset of the genetic markers (i.e., a “haplotype tag”) can be chosen that uniquely identifies each of these haplotypes.

The term “heterosis” refers to hybrid vigor, i.e., the improved or increased function of any biological quality (e.g., size, growth rate, fertility, yield, etc.) in a hybrid offspring relative to its parents. For example, the offspring of a cross between inbred plant lines from different heterotic groups is likely to display more heterosis than its parent lines, as described above. The first-generation offspring of such a cross generally show, in greater measure, the desired characteristics of both parents. This heterosis may decrease in subsequent generations if the first-generation hybrids are mated together.

The terms “heterotic group” and “heterotic pool” are used interchangeably and refer to a group of genotypes or inbred lines that demonstrate similar heterotic response when crossed with genotypes or inbred lines from other genetically distinct germplasm groups. There is a closer degree of genetic relationship of lines contained within a heterotic group versus the more distant degree of genetic relationship of lines compared between heterotic groups. In general, the hybrid of two inbred lines crossed together within the same heterotic group shows much less heterosis than the hybrid of an inbred line from one heterotic group crossed to an inbred line from a different heterotic group. A particular heterotic group can include multiple lines having diverse genetics.

The terms “hybrid”, “hybrid plant”, and “hybrid progeny” in the context of plant breeding refer to a plant that is the offspring of genetically dissimilar parents produced by crossing plants of different lines or breeds or species, including but not limited to the cross between two inbred lines (e.g., a genetically heterozygous or mostly heterozygous individual). The phrase “single cross FI hybrid” refers to an FI hybrid produced from a cross between two inbred lines.

The phrase “inbred line” refers to a genetically homozygous or nearly homozygous population. An inbred line, for example, can be derived through several cycles of brother/sister breedings or of selfing. In some embodiments, inbred lines breed true for one or more phenotypic traits of interest. An “inbred”, “inbred individual”, or “inbred progeny” is an individual sampled from an inbred line. The term “inbred” means a substantially homozygous individual or line. An inbred line may also be referred to as a “parent line” when used in a breeding program.

The terms “introgression”, “introgressed”, and “introgressing” refer to both a natural and artificial process whereby genomic regions of one species, variety or cultivar are moved into the genome of another species, variety or cultivar, by crossing those species. The process may optionally be completed by backcrossing to the recurrent parent.

The term “loss-of-function mutation” is a change in the DNA sequence of a gene (i.e., a “mutation”) that results in the mutated gene product lacking the molecular function of the wild-type gene. There are four main genetic variations that can lead to loss-of-function mutations: 1) a mutation resulting in a premature stop codon producing a truncated protein sequence; 2) a mutation occurring at a canonical splice site that affects splicing (resulting in inclusion of an intron or exclusion of an exon in the mRNA transcript); 3) an insertion or deletion variant with non-integral multiples of three located in the gene coding region, causing frameshifts by disrupting the full-length transcript; and 4) mutations that result in the loss of an initiation codon (transcription start codon, e.g. ATG), which prevent gene transcription if there is no alternative start codon near the mutation. In addition, mutations in the promoter or untranslated regions (UTRs) of a gene can reduce or eliminate gene expression, leading to a loss-of-function. The term “knockout mutation” is used interchangeably.

The terms “nucleic acid” and “polynucleotide” are used interchangeably and as used herein refer to both sense and anti-sense strands of RNA, cDNA, genomic DNA, mitochondrial DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA and/or RNA. In addition, a polynucleotide disclosed herein, e.g., a circular DNA template, a nucleic acid concatemer disclosed herein, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Nucleotide sequences are “complementary” when they specifically hybridize in solution (e.g., according to Watson-Crick base pairing rules). The term also includes codon-optimized nucleic acids that encode the same polypeptide sequence. It is also understood that nucleic acids can be unpurified, purified, or attached, for example, to a synthetic material such as a bead or column matrix.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “offspring” plant refers to any plant resulting as progeny from a vegetative or sexual reproduction from one or more parent plants or descendants thereof. For instance, an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offsprings of second generation (F2) or subsequent generations (F3, F4, etc.) are specimens produced from selfings of F1s, F2s etc. An F1 may thus be a hybrid resulting from a cross between two true breeding parents, while an F2 may be an offspring resulting from self-pollination of said F1 hybrids.

The term “PCR (polymerase chain reaction)” is understood within the scope of the invention to refer to a method of producing relatively large amounts of specific regions of DNA, thereby making possible various analyses that are based on those regions.

“Phenotype” is understood within the scope of the present disclosure to refer to a distinguishable characteristic(s) of a genetically controlled trait. The phrase “phenotypic trait” refers to the appearance or other detectable characteristic of an individual, resulting from the interaction of its genome with the environment.

A “plant” is any plant at any stage of development, particularly a seed plant. In particular, in the context of this disclosure, a plant refers to a maize plant.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

The term “population” means a genetically heterogeneous collection of plants sharing a common genetic derivation.

As used herein, the term “primer” refers to an oligonucleotide that is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

The term “random mutagenesis” refers to alternative methods of mutation. In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See McCallum, et al., Targeted screening for induced mutations, NAT. BIOTECHNOL. 18 (4): 455-57 (2000) and McCallum, et al., Targeting induced local lesions IN genomes (TILLING) for plant functional genomics, PLANT PHYSIOL. 123 (2): 439-42 (2000).

The phrases “sexually crossed” and “sexual reproduction” in the context of the present disclosure refer to the fusion of gametes to produce progeny (e.g., by fertilization, such as to produce seed by pollination in plants). In some embodiments, a “sexual cross” or “cross-fertilization” is fertilization of one individual by another (e.g., cross-pollination in plants). In some embodiments the term “selfing” refers to the production of seed by self-fertilization or self-pollination; i.e., pollen and ovule are from the same plant.

The terms “solvent” and/or “solution” refer to a liquid in which the flavonoid can be mixed. Examples include water, alcohol, propylene glycol, DMSO, Tris buffer, and other liquids available to a person skilled in the art. The flavonoid need not be dissolved in the liquid; a mixture or emulsion can be sufficient.

“Transformable,” “transformability,” and the like, refers to a plant, a line of plants, or a plant cell (such as callus tissue or a protoplast) that is more readily accepting of foreign DNA and can stably integrate the foreign DNA into its genome.

The terms “variety” or “cultivar” mean a group of similar plants that by structural or genetic features and/or performance can be distinguished from other varieties within the same species.

### DETAILED DESCRIPTION

An embodiment of the invention is a method of seed production, comprising: (a) obtaining at least one first plant, wherein the first plant is a flavonoid-dependent sterility (“FDF”) plant comprising a mutation in an FDF gene; (b) obtaining at least one second plant, wherein the second plant is male fertile; (c) allowing pollination by the at least one second plant to the at least one first plant; and (d) obtaining progeny seed thereof. In one aspect, the first plant and the second plant are monocot plants. In a further aspect, the monocot plants are selected from the group consisting of wheat, maize, and rice. In another aspect, the FDF gene is selected from the group consisting of SEQ ID NOs: 1-5 and 57.

Another embodiment of the invention is a flavonoid-dependent fertile (“FDF”) plant comprising a mutation in an FDF gene. In one aspect, the mutation in the FDF gene is a knock-out mutation. In another aspect, the plant is homozygous for the knock-out mutation in the FDF gene. In one aspect, the FDF plant is a wheat plant and the FDF gene is selected from TaFDF1A (SEQ ID NO: 1), TaFDF1B (SEQ ID NO: 2), TaFDF1D (SEQ ID NO: 3), and a combination thereof. In another aspect, the FDF plant is a maize plant and the FDF gene is ZmFDF1 (SEQ ID NO: 4) or ZmFDF2 (SEQ ID NO: 5) or both. In a further aspect, the FDF plant further comprises a mutation in C2/WHP. In another aspect, the FDF plant is a rice plant, and the FDF gene is OsFDF (SEQ ID NO: 57).

Another embodiment of the invention is a method of propagating an FDF plant, comprising: (a) obtaining at least one plant, wherein the plant is a flavonoid-dependent fertile (“FDF”) plant comprising a mutation in an FDF gene; (b) applying a composition comprising a flavonoid to the plants of step (a); (c) allowing self-pollination to occur; and (d) obtaining progeny seed thereof. In one aspect, the flavonoid is quercetin. In another aspect, the composition comprising quercetin is a liquid solution. In another aspect, the liquid solution comprises quercetin mixed in water, propylene glycol, or other solvent or solution. In yet another aspect, the liquid solution comprises quercetin suspended in a solvent or solution at a concentration of 1 mg/L to 100 mg/L, 2.5 mg/L to 50 mg/L, or approximately 5 mg/L. In still another aspect, the liquid solution comprising quercetin is saturated. In an alternative aspect, the composition comprising quercetin is a powder. In one aspect, the powder comprises a mixture of quercetin and a carrier compound. In one aspect, the mixture of quercetin and carrier compound is at a ratio between 1000:1 to 1:1000. In another aspect, the carrier compound is selected from the group consisting of crystalline silica, talc, metallic powder, and mica minerals. In still another aspect, the composition comprising a flavonoid is applied to female organs of a flower on the at least one plant designated as a female plant. In yet another aspect, the composition is applied by a method selected from the group consisting of painting, misting, spraying, and root drenching. In one aspect, the FDF gene is a a wheat gene selected from the group consisting of SEQ ID NOs: 1-3 and a combination thereof. In another, the FDF gene is a maize gene selected from the group consisting of SEQ ID NOs: 4-5 and a combination thereof. In yet another, the FGF gene is a rice gene comprising SEQ ID NO: 57. One embodiment of the invention is a plant made by the method recited above.

Another embodiment of the invention is a method of hybrid seed production, comprising: (a) obtaining at least one inbred female FDF plant, wherein the inbred female FDF plant comprises a mutation in an FDF gene; (b) obtaining pollen from at least one inbred male plant, wherein the inbred male plant produces fertile pollen; (c) pollinating the inbred female FDF plant with the fertile pollen from the inbred male plant; and (d) obtaining hybrid progeny thereof. Another embodiment of the method is a plant made by the previous methods. In one aspect, the inbred female FDF plant is a maize plant, a wheat plant, or a rice plant. In another aspect, the FDF wheat plant comprises a mutation in a gene selected from the group consisting of SEQ ID NOs: 1-3, and a combination thereof. In yet another aspect, the FDF maize plant comprises a mutation in a gene selected from the group consisting of SEQ ID NOs: 4-5 and a combination thereof. In still another aspect, the FDF rice plant comprises a mutation in SEQ ID NO: 57. An embodiment of the invention is a plant made by the method recited above.

Another embodiment of the invention is a method of restoring fertility to an FDF plant comprising a mutation in an FDF gene, the method comprising applying a composition comprising a flavonoid to the FDF plant. In one aspect, the FDF plant is a maize plant, a wheat plant, or a rice plant. In another aspect, the FDF wheat plant comprises a mutation in a gene selected from the group consisting of SEQ ID NOs: 1-3, and a combination thereof. In yet another aspect, the FDF maize plant comprises a mutation in a gene selected from the group consisting of SEQ ID NOs: 4-5 and a combination thereof. In still yet another aspect, the FDF maize plant further comprises a mutation in a chalcone synthase gene. In yet another aspect, the FDF rice plant comprises a mutation in SEQ ID NO: 57. In one aspect, the flavonoid comprises quercetin. In another aspect, the quercetin is mixed in water, propylene glycol, or other solvent or solution. In an alternative aspect, the composition comprising a flavonoid further comprises a carrier compound. In an aspect, the carrier compound is selected from the group consisting of crystalline silica, talc, metallic powder, and mica minerals. In one aspect, the composition comprising a flavonoid is applied to anthers, silks, stigmas, florets, spikes, leaf whorls, leaf canopy, or roots. In an aspect, the composition is applied by a method selected from the group consisting of painting, misting, spraying, and root drenching.

### EXAMPLES

### 1. Identifying FDF Genes in Maize.

Anther-specific chalcone synthase genes were originally identified in maize during research into the causative mutations underlying the publicly available colorless2 (c2) and whitepollen1 (whp1) alleles in the maize mutant line, 224H. In the 224H line, whitepollen1 is homozygous for the recessive whp1 mutant allele and segregating for the mutant colorless2 allele, c2. The male sterility phenotype is observed in individual plants homozygous for c2, while plants heterozygous or homozygous for C2 are male fertile. In the 224H line, zygosity of colorless2 is visually tracked by the presence or absence of purple aleurone. An embryo homozygous or heterozygous for wild type C2 will be capable of synthesizing anthocyanins in the kernel aleurone which will result in a visually purple kernel. Embryos homozygous for mutant c2 are not able to synthesize anthocyanins in the kernel aleurone, resulting in colorless aleurone that reveals the colorless (white) endosperm below (in line 224H the endosperm is always white; endosperm color is controlled by an independent, unrelated gene, Y1). While these mutant alleles have been known to the maize research community, we were not able to identify a source publishing the causative mutations underlying these mutant alleles. To identify the causative mutation underlying colorless2, we planted a population of 224H seed segregating for c2 (mutant) and C2 (wild type). Tissue samples were collected and the entire colorless2 gene was amplified using sets of PCR primers. PCR products were sequenced by Sanger sequencing. A SNP haplotype (Table 1) variant that differed from the C2 in the B73, NP2222, and W22 reference genomes was identified as segregating 1:2:1 in the population of 224H plants. Multiple seedlings derived from white kernels were found to be homozygous for the SNP haplotype. The whp1 allele was also amplified by PCR primers and PCR products were sequenced by Sanger sequencing for comparison to the B73, AX5707, and W22 reference genomes. No putative causal mutations the whp1 mutant allele were identified.

TABLE 1
Haplotype identified in the c2 allele. Individual 224H plants
found to be male sterile were homozygous for this haplotype.

| Position in | | | | |
|---|---|---|---|---|
| Zm00001d052673 | Gene | C2 | c2 | Resulting Amino |
| Gene Model | Region | Sequence | Sequence | Acid Substitution |
| 1887-1888 | Intron | CA | 2 bp | NA |
| | | | deletion | |
| 1928 | Intron | T | G | NA |
| 1973 | Exon 2 | C | T | R72C |

NA = Not Applicable

Further investigation into the c2 SNP haplotype focused on the C1973T SNP that resulted in an R72C amino acid substitution. This amino acid substitution is not part of the chalcone synthase catalytic triad. Without being bound by theory, a radical amino acid substitution from the positively charged side chain on arginine to the thiol side chain on cysteine would likely have a deleterious impact on protein function. The first evidence in favor of the C1973T SNP being causative was found in publications detailing that the arginine at amino acid position 72 in C2 was part of a conserved motif required for coenzyme A binding in chalcone synthase (see, e.g., J-L Ferrer, et al., Structure of chalcone synthase and the molecular basis of plant polyketide biosynthesis, NATURE STRUCTURAL BIOL. 6 (8): 775-784 (1999) and M. B. Austin & J. P. Noel, The chalcone synthase superfamily of type III polyketide synthases, NAT. PROD. REP. 20:79-110 (2003)). Additional evidence was identified by comparing the variant c2 amino acid sequence containing the R72C substitution to the wild type C2 sequence using the PROVEAN software (Y. Choi & A. P. Chan, PROVEAN web server: a tool to predict the functional effect of amino acid substitutions and indels, BIOINFORMATICS 31 (16): 2745-2747 (2015).). The software returned a score of −6.055 for the R72C amino acid substitution and predicted that this mutation would be deleterious to protein function. Finally, the project team identified that a similar amino acid substitution had been identified in a Matthiola incana mutant line (V. Hemleben, et al., Characterization and structural features of a chalcone synthase mutation in a white-flowering line of Matthiola incana R. Br. (Brassicaceae), PLANT MOL. BIOL. 55:455-465 (2004)). Hemleben, et al. characterized an R72S amino acid substitution that eliminated chalcone synthase protein activity and resulted in a colorless flower phenotype in Matthiola incana, further highlighting that a substitution at the R72 position will result in loss of function and subsequent inability to synthesize the flavonoids required for pigment synthesis.

Based on identification of a causative mutation for the c2 mutant allele in maize, we began to search for an ortholog of c2 in wheat that could be targeted using gene editing, for example but not limited to CRISPR-based editing. During this search in wheat, additional chalcone synthase genes in maize were identified, including chalcone synthase2 (chls2) GRMZM2G380650 (SEQ ID NO: 4). Inclusion of chls2 in the analysis led the project team to identify additional chalcone synthase genes with anther specific expression patterns (TraesCS1A02G160300 (SEQ ID NO: 1), TraesCS1B02G176300 (SEQ ID NO: 2), and TraesCS1D02G157500 (SEQ ID NO: 3)). These additional chalcone synthase genes were found to have anther-specific expression patterns and were included as candidate genome editing targets to generate FDS in wheat. In maize there are 6 chalcone synthases that catalyze the same enzymatic step in the flavonoid biosynthetic pathway, (this step is designated EC 2.3.1.74), according to the Kyoto Enzyme Gene and Genomes (KEGG) database (www.genome.jp/kegg/annotation/enzyme.html). These 6 chalcone synthases include C2, WHP1, and CHLS2. To determine if orthologues other than CHLS2 should be included as candidates for editing to create the FDF system in maize, the expression pattern across cell tissues was analyzed using RNA-seq data from a range of developmental stages in 2 datasets: the RNA expression hosted publicly at MaizeGDB.org and an internal RNA-seq atlas. The qualitative results for the 6 maize genes that catalyze EC step 2.3.1.74 are summarized in Table 2; four of these genes (C2, WHP1, CHLS2, and CHLS11) all had very high expression in anther tissues. A BLAST search showed that CHLS11 was found to be the closest hit to CHLS2, with 92% sequence identify at the nucleotide level, and even higher when only the coding sequences are compared. Because of the expression pattern, enzymatic profile, and sequence similarity, CHLS11 was also included as a potential editing candidate for maize FDF.

TABLE 2
Maize gene identifiers and summary of relative expression for CHS genes
that catalyze enzymatic step 2.3.1.74 in flavonoid biosynthesis

| | | | | | Qualitative |
|---|---|---|---|---|---|
| | | | | | Relative |
| | | | | | Expression |
| | | | | | Intensity |
| | | | Chromosome | | (High, Med, |
| | | | Coordinates | | Low) and |
| GRMZM ID | | Chromosome | (B73 | Gene | Tissue- |
| Number | Zm Gene ID | Number | RefGen_v4) | Name | Specificity |
| GRMZM2G009348 | Zm00001d052676 | Chr4 | 192838961- | chls7 - | High: silks; |
| | | | 192840700 | chalcone | Med: |
| | | | | synthase7 | youngest fully |
| | | | | | mature leaf, |
| | | | | | primary ear, |
| | | | | | cob, spikelets |
| GRMZM2G131529 | Zm00001d040483 | Chr3 | 46573878- | chls12 - | Med: |
| | | | 46575942 | chalcone | Anthers; |
| | | | | synthase 12 | Low: |
| | | | | | Spikelets, |
| | | | | | youngest fully |
| | | | | | mature leaf |
| GRMZM2G151227 | Zm00001d007403 | Chr2 | 224558005- | whp1 - | Very High: |
| | | | 224561990 | white | spikelet, |
| | | | | pollen1 | anthers, silks, |
| | | | | | stem |
| | | | | | internode, |
| | | | | | youngest fully |
| | | | | | mature leaf |
| | | | | | middle; Med: |
| | | | | | most other |
| | | | | | places |
| GRMZM2G380650 | Zm00001d032662 | Chr1 | 229958886- | chls2 - | Very High: |
| | (ZmFDF1) | | 229962459 | chalcone | spikelet, |
| | | | | synthase2 | anthers; High: |
| | | | | | Most other |
| | | | | | places |
| GRMZM2G422750 | Zm00001d052673 | Chr4 | 192758391- | c2 - | Very High: |
| | | | 192761788 | colorless2 | spikelet, |
| | | | | | anthers, silks, |
| | | | | | primary ear, |
| | | | | | stem |
| | | | | | internode; |
| | | | | | High: leaf, |
| | | | | | root, cob |
| GRMZM2G477683 | Zm00001d013991 | Chr5 | 26991391- | chls11 - | Very High: |
| | (ZmFDF2) | | 26995651 | chalcone | spikelet, |
| | | | | synthase11 | anthers |

### 1A. Identifying Candidate Genes for FDF in Rice

After identification of Zmchls2 and Zmchls11 as targets for FDF, we sought to identify chalcone synthase genes in rice that could be targets for creation of an FDF system. In rice (Oryza sativa), there is only a single orthologue to ZmC2 and ZmWHP: OsCHS1 (Os11g0530600). Mutation in OsCHS1 has been previously reported to be male sterile (Wang, Lanxiang, Pui Ying Lam, Andy C. W. Lui, Fu-Yuan Zhu, Mo-Xian Chen, Hongjia Liu, Jianhua Zhang, and Clive Lo. 2020. “Flavonoids Are Indispensable for Complete Male Fertility in Rice.” Journal of Experimental Botany 71 (16): 4715-28. doi.org/10.1093/jxb/eraa204). However, no reference to the orthologue(s) of ZmCHLS2 and ZmCHLS11 has been previously reported. Using a BLAST search to compare genomic DNA sequences, spliced RNA sequence, and protein amino acid sequences between the corn ZmCHLS2 and ZmCHLS11 and the rice genome (NCBI, genome assembly IRGSP-1.0) the best match is rice gene OsFDF with 85% sequence similarity to the maize orthologues (Table 3).

TABLE 3
Sequence similarities between maize chalcone
synthase orthologues and OsFDF.

| | Genomic DNA | Spliced | Amino Acid |
|---|---|---|---|
| | Sequence | Sequence | Sequence |
| Zm Gene Name | Similarity | Similarity | Similarity |
| ZmCHLS2 | 85% | 89% | 85% |
| ZmCHLS11 | 85% | 79% | 85% |

Rice gene OsFDF is not assigned to any metabolic step in the KEGG database, so it is not clear if it catalyzes KEGG enzymatic step 2.3.1.74, the step catalyzed in flavonoid biosynthesis by maize genes ZmC2, ZmWHP1, ZmCHLS2, and ZmCHLS11. But it is predicted to be a “chalcone and stilbene synthase” by Uniprot based on protein domains and structure (www.uniprot.org/uniprotkb/Q7X795/entry); this is the same protein class the maize FDF genes are assigned to. There are 4 other rice genes predicted to catalyze step 2.3.1.74 (Table 4), however when their expression patterns were compared with OsFDF in an internal RNA-seq dataset, only 1 of these 4 genes exhibited the high, male-reproductive-tissue-enriched expression pattern required for FDF (Os07g0411300). OsFDF is most strongly expressed in male tissues (Table 4). The combination of its sequence similarity, the presence of chalcone stilbene synthase protein domains, and the necessary male-enriched gene expression pattern make OsFDF an ideal candidate for mutation to establish an FDF system in rice.

TABLE 4
Relative tissue expression patterns of Oryza sativa chalcone synthase genes

| | | | | | | Qualitative |
|---|---|---|---|---|---|---|
| | | | | | | Relative |
| | | | Os Gene | | | Expression |
| | | | Name (Rice | | | Intensity (High, |
| | SEQ | | Annotation | KEGG | | Med, Low) and |
| | ID | FDF | Project | Pathway | Chromosome: | Tissue- |
| Os Gene ID | NO: | Candidate | Database) | Step | Coordinates | Specificity |
| Os05g0212900 | 53 | NO | OsCHS6 | 2.3.1.74 | 5: 6993662- | High: Leaf |
| | | | | | 6991465 | maturation and |
| | | | | | | elongation |
| | | | | | | zones, root tip; |
| | | | | | | Medium: |
| | | | | | | Seedling, radicle |
| | | | | | | tip, roots |
| Os07g0214900 | 54 | NO | OsCHS2, | 2.3.1.74 | 7: 6296305- | Medium: Root |
| | | | OsCHS8 | | 6294479 | tip, roots, |
| | | | | | | seedling |
| Os07g0501100 | 55 | NO | OsCHS12 | 2.3.1.74 | 7: 18875926- | Medium: Root |
| | | | | | 18873790 | tip |
| Os11g0530600 | 56 | YES | OsCHS1, | 2.3.1.74 | 11: 19281006- | High: Leaf, |
| | | | OsCHS24 | | 19277597 | shoot, panicle, |
| | | | | | | seedling, flag |
| | | | | | | leaf; Medium: |
| | | | | | | Anther, root tip, |
| | | | | | | coleoptile, pistil, |
| | | | | | | embryo, roots, |
| | | | | | | zygote |
| Os10g0484800 | 57 | YES | OsPKS1, | Not | 10: 18316329- | High: Anther; |
| (“OsFDF”) | | | OsLAP6 | assigned | 18318605 | Medium: Ovary |
| | | | | | | wall, pistil, |
| | | | | | | ovule, embryo, |
| | | | | | | spikelet, panicle |

### 2. Wheat Transformation.

A binary vector that would generate loss of function alleles at the TraesCS1A02G160300, TraesCS1B02G176300, and TraesCS1D02G157500 genome editing targets was designed. To generate double stranded cuts in the targeted genes, a wheat codon optimized version of the Cas9 enzyme derived from Streptococcus pyogenes was selected. This version of Cas9 contained L1181V and L1196V amino acid substitutions and a nuclear localization sequence at both the N-terminal and C-terminal ends of amino acid sequence. Cas9 transcription was driven by a constitutive sugarcane ubiquitin 4 promoter derived from Saccharum officinarum. This promoter was enhanced by three elements; a nopaline synthase enhancer derived from Agrobacterium tumefaciens, a figwort mosaic virus enhancer derived from Figwort mosaic caulimovirus, and a cauliflower mosaic virus 35S enhancer region derived from Cauliflower mosaic caulimovirus. A terminator sequence based on a maize metallothionin-like gene was included after Cas9 to terminate transcription.

Two separate dual-guide RNAs targeting the first exon in TraesCS1A02G160300, TraesCS1B02G176300, and TraesCS1D02G157500 were designed based on a consensus sequence derived from Chinese Spring wheat. One guide RNA targeted the fourth codon in each of the three chalcone synthase gene targets and was designated as TaGHSGTarget1. The second guide RNA targeted the fifty-first codon in each of the three chalcone synthase gene targets and was designated as TaGHSGTarget2. Guide RNA transcription was driven by a constitutive sugarcane ubiquitin 4 promoter derived from Saccharum officinarum with no enhancer elements. For efficient guide RNA processing, TaGHSGTarget1 and TaGHSGTarget2 were separated from the trans-activating crRNA sequences derived from Streptococcus pyogenes to by a HammerHead RNA sequence. After transcription, these units form a combined guide RNA and crRNA sequence for each of the two guide RNAs (this is herein referred to as a “dual-guide” system). These combined guide RNA and crRNA sequences were designated as rCrRNATaCHSG-01 and rCrRNATaCHSG-02. Additional sequences of trans-activating crRNA were included in the design and were designated as rTracrRNA. To further facilitate efficient guide RNA processing, the design included multiple copies of a self-cleaving hammerhead RNA derived from Tobacco etch potyvirus, designated as rHH, and hammerhead elements with pairing sequences specific to each guide RNA and crRNA complex, designated as rHHTaCHSG-01 and rHHTaCHSG-02, respectively. Multiple copies of self-cleavable ribozyme derived from Hepatitis delta virus, designated as rHDV. These elements were linked together into a continuous sequence in order of: rHH, rTracrRNA, HDV, rHH, rHH, rTracrRNA, rHV, rHH, rHHTaCHSG-01, rCrRNATaCHSG-01, rHDV, rHHTaCHSG-02, rCrRNATaCHSG-02, rHDV. Transcription of this guide RNA and processing element complex was terminated by a nopaline synthase terminator derived from Agrobacterium tumefaciens.

Phosphomannose-isomerase (PMI; see U.S. Pat. No. 5,767,378) was chosen as the selectable marker for stable plant transformation. PMI transcription was driven by a ubiquitin promoter and transcription was terminated by a ubiquitin terminator, both derived from Zea mays. The Cas9 expression cassette, guide RNA expression and processing cassette, and PMI selectable marker cassette were linked to a standard binary vector containing left and right border elements derived from Agrobacterium tumefaciens and backbone elements. Backbone elements included a copy of the aminoglycoside 3′adenyltransferase gene that confers resistance to spectinomycin and streptomycin for maintenance of the vector in E. coli and Agrobacterium, derived from Escherichia coli. The backbone also included a bacterial expression cassette of two Agrobacterium tumefaciens genes driven by the VirG promoter, including the VirG transcriptional activator virulence factor and the RepA plasmid portioning protein. The backbone included the oVS1 origin of replication element derived from Pseudomonas sp. for plasmid replication in Agrobacterium tumefaciens and the ColE1 origin of replication element for plastic replication in Escherichia coli. All elements described above were assembled into a single binary vector in September and October 2019, validated by sequencing, and designated as Syngenta construct 25206.

Syngenta construct 25206 was stably transformed into the Fielder wheat variety using Agrobacterium-mediated plant transformation. Thirty-four TO wheat plants were generated from transformation experiments. All TO plants were allowed to self-pollinate to produce T1 seed. T1 seed lots from low-copy number TO parents were planted and screened for editing activity at the TraesCS1A02G160300, TraesCS1B02G176300, and TraesCS1D02G157500 target sites. Screening was accomplished by amplifying the region of each gene around the rCrRNATaCHSG-01 and rCrRNATaCHSG-02 cutting sites using PCR and sequencing the resulting PCR products. Primers used for PCR amplification are described in Table 5. PCR conditions for all primers used an annealing of 66 °C, a 60 second extension time, and 40 cycles of amplification. This PCR screening identified edits at the rCrRNATaCHSG-01 cutting site in TraesCS1A02G160300 and TraesCS1B02G176300 in separate T1 plants. All genome edits in this study were generated at the rCrRNATaCHSG-01 cutting site and no evidence of editing was found at the rCrRNATaCHSG-02 cutting site throughout the duration of the study. Pollen was collected from the T1 plant containing an edit at the TraesCS1B02G176300 target and was used to pollinate two T1 plants with edits at the TraesCS1A02G160300 target. The T2 seed produced by this cross between sibling T1 plants was planted and screened for edits at all targets using the same methods. This screening confirmed that edits at TraesCS1A02G160300 and TraesCS1B02G176300 had been combined into one T2 plant and that a sperate T2 sibling plant had both the edit at the TraesCS1A02G160300 target and a new edit at the rCrRNATaCHSG-01 cutting site within TraesCS1D02G157500. The T2 plant with genome edits to the TraesCS1A02G160300 and TraesCS1B02G176300 editing targets was self-pollinated for phenotyping in the T3 generation. T3 progeny of this plant were selected for individuals homozygous for the genome edits at TraesCS1A02G160300 and TraesCS1B02G176300, grown to maturity, and observed for male fertility. No evidence of male sterility or alterations to plant phenotype were observed in individuals homozygous for genome edits at TraesCS1A02G160300 and TraesCS1B02G176300. It is later demonstrated that the functional wild type allele of TraesCS1D02G157500 was sufficient to maintain complete male fertility.

TABLE 5
PCR primers and conditions.

| | | | | primer_pairs: | |
|---|---|---|---|---|---|
| | | | | annealing temp | |
| SEQ | | | | (C), | |
| ID | | | | product size | |
| NO: | Ref | primer | primer_sequence | (bp) | primer_annotation |
| 19 | Chr1A | LZ311 | TCAGTTTACACTCATGCCG | 311-312: 66 | Forward primer to PCR Chr1A |
| | | | | degree, 495 bp | gene editing in wheat |
| 20 | Chr1A | LZ312 | CGAAGTCGACAGAGAACAAC | 311-312: 66 | reverse primer to PCR Chr1A |
| | | | | degree, 495 bp | gene editing in wheat, product |
| | | | | | size 495 bp. |
| 21 | Chr1B | LZ313 | GGTTTTCACTCACACCGTCG | 313-314: 66 | Forward primer to PCR Chr1B |
| | | | | degree, 636 bp | gene editing in wheat |
| 22 | Chr1B | LZ314 | GTTACAACCAGATGGCAACC | 313-314: 66 | reverse primer to PCR Chr1B |
| | | | | degree, 636 bp | gene editing in wheat, product |
| | | | | | size 636 bp. |
| 23 | Chr1D | LZ315 | TGGGAGAACAAACCTGCGC | 315-316: 66 | Forward primer to PCR Chr1D |
| | | | | degree, 578 bp | gene editing in wheat |
| 24 | Chr1D | LZ316 | TTTCGAAGTCGACGGAGAAC | 315-316: 66 | reverse primer to PCR Chr1D |
| | | | | degree, 578 bp | gene editing in wheat, product |
| | | | | | size 578 bp. |

Pollen from a T2 segregant with the genome edit to TraesCS1B02G176300 was used to pollinate the individual with edits to the TraesCS1A02G160300 and TraesCS1D02G157500 targets. The T3 progeny of this cross were planted, screened by PCR and sequencing, and individuals were selected with a genome edit at the rCrRNATaCHSG-01 cutting site in all three targets. Selected T3 progeny were allowed to naturally self-pollinate to produce a T4 generation segregating for individuals with homozygous edits at all three targets. The T4 generation was planted and screened by PCR product sequencing. Segregation distortion was observed in the T4 generation due to poor transmittance of pollen with edits to all three targets (Table 6). One individual T4 plant was identified as having homozygous loss of function edits to all three target sites (Table 6). Additional T4 plants with knockout edited alleles at five of the possible six target copies (six is the total copy number from the three diploid A, B, and D wheat genomes) were selected for phenotyping and use as pollen donors. Additional T4 plants included individuals with one functional copy of TraesCS1B02G176300 and individuals with one functional copy TraesCS1D02G157500. Knockout edited alleles at the TraesCS1A02G160300 target were fixed homozygous in this line by the T4 generation. All edits sequenced from individuals in the T4 generation were those described in Table 6.

TABLE 6
Comparison of expected and observed segregation ratios for the three editing targets in
T4 seedlings. All 192 seedlings screened were the bulked self-pollination progeny of an
individual T3 plant homozygous for the TraesCS1A02G160300 edited allele and heterozygous
for both the TraesCS1B02G176300 and TraesCS1D02G157500 edited alleles. Sequencing confirmed
that edited alleles in all 192 T4 progeny screened were those described in Table 6.

| | Total | Expected Segregation | | | Observed Segregation | | | |
|---|---|---|---|---|---|---|---|---|
| | Progeny | Hom | Het | Hom | Hom | Het | Hom | Chi Square |
| Editing Target | Screened | Edited | Edited | WT | Edited | Edited | WT | Test |
| TraesCS1A02G160300 | 192 | 192 | 0 | 0 | 192 | 0 | 0 | NA |
| TraesCS1B02G176300 | 192 | 48 | 96 | 48 | 15 | 107 | 70 | 4.08 × 10\hat{ }⁻⁰⁸ |
| TraesCS1D02G157500 | 192 | 48 | 96 | 48 | 43 | 106 | 43 | 0.35 |

NA = Not Applicable

TABLE 7
Edited alleles generated at anther-specific chalcone synthase targets
in Triticum aestivum variety Fielder. All deletions occurred
in the fourth codon of exon one the coding sequence and resulted
in a frameshift that impacted all downstream codons.

| Editing Target | Indel Type | Edited SEQ ID NO: |
|---|---|---|
| TraesCS1A02G160300 | 1 bp deletion | 6 |
| TraesCS1B02G176300 | 2 bp deletion | 7 |
| TraesCS1D02G157500 | 1 bp deletion | 8 |

### 3. Phenotyping Homozygous Edited Wheat Plants.

The phenotype at flowering of the single T4 plant homozygous for knockout edited alleles at all six copies of the editing targets shared similarities to phenotypes commonly observed in timopheevii CMS wheat. See, e.g., J. Song, A chimeric gene (orf256) is expressed as protein only in cytoplasmic male-sterile lines of wheat, PLANT MOL. BIOL. 26:535-539 (1994). Outer florets on individual spikes extruded yellow anthers during flowering. These anthers were observed to not dehisce along the lodicules and did not shed pollen as is observed in fertile wheat. Several days after the start of anther extrusion, secondary gaping was observed during which all individual florets opened to enable outside pollen access to stigmas. In contrast to timopheevii CMS wheat, all directly observed florets had the expected three fully formed anthers per floret and the anthers themselves were similar in size to male fertile Fielder plants. No instances of underdeveloped or misshapen anthers commonly observed in timopheevii CMS wheat were recorded. Six spikes showing secondary gaping were pollinated using pollen from sibling segregant plants with one intact copy of the anther specific chalcone synthase (TraesCS1B02G176300 or TraesCS1D02G157500). All individuals with one intact copy of the anther specific chalcone synthase demonstrated normal phenotypes throughout their life cycle and normal seed set at harvest; further, individuals comprising one copy of the functional anther specific chalcone synthase were all observed to shed normal quantities of fertile, yellow pollen during pollen collection. No differences were observed between individuals with a single intact copy of TraesCS1B02G176300 and those with a single intact copy of TraesCS1D02G157500.

TABLE 8
Seed set outcomes from self-pollination and cross-pollinations between siblings
in a population of Triticum aestivum segregating for genome edited loss-of-function
alleles at TraesCS1A02G160300, TraesCS1B02G176300, and TraesCS1D02G157500.

| | Female Parent | | Count of Spikes | Total Seed |
|---|---|---|---|---|
| Pollination Type | Genotype | Male Parent Genotype | Harvested (#) | Count (#) |
| Cross | aabbdd | aaBbdd | 3 | 4 |
| Cross | aabbdd | aabbDd | 3 | 66 |
| Self | aabbdd | aabbdd | NA | 1 |
| Self | aaBbdd | aaBbdd | NA | 1633 |
| Self | aaBbdd | aaBbdd | NA | 1228 |
| Self | aaBbdd | aaBbdd | NA | 1998 |
| Self | aabbDd | aabbDd | NA | 1646 |
| Self | aabbDd | aabbDd | NA | 1724 |
| Self | aabbDd | aabbDd | NA | 1005 |

NA = Not applicable; count of spikes harvested was not recorded for instances where all spikes from a single plant were threshed as a single bulk.
Uppercase letter = intact WT allele, lowercase letter = loss of function edited allele

### 4. Fertility Restoration in Wheat

It is hypothesized that application of exogenous flavonoids to the developing wheat anthers will restore male fertility in full knockout plants (aabbdd genotype). Male fertility restoration treatments may be applied to individual spikes on plants homozygous for knockout edited alleles at all three targets. Treatments may consist of quercetin exogenously applied at one or multiple microspore development stages. Quercetin may be applied exogenously as a dry powder, dissolved in a solvent or solution, or suspended in a formulation. Liquid solutions and suspensions of quercetin dihydrate may include inert ingredients that enhance the penetrance of quercetin into plant tissues. Liquid and dry powdered treatments may be applied to stigmas within individual florets of the wheat spike, the entire wheat spike, the entire tiller from which a spike originates, or the entire plant through soaking or drenching.

### 5. Maize Transformation.

Six guide RNA sequences (gRNAs) targeting the second exon in ZmFDF1 and ZmFDF2 for gene editing were designed based on genomic sequence from line NP2222v5. The design and validation of these gRNAs was conducted by Syngenta in September-November 2021, prior to vector design and assembly. These gRNAs were designed by identifying all the possible PAM recognition sites (TTTN for LbCas12a) in the gene coding sequences and then selecting the guides that were closest to the transcription start site of the target gene, least likely to make any off-target edits (i.e. fewest potential sequence matches elsewhere in the genome particularly in other chalcone synthase genes), and likely to disrupt the conserved catalytic triad in exon 2 known to be essential for chalcone synthase function across several species (M. B. Austin & J. P. Noel, The chalcone synthase superfamily of type III polyketide synthases, NAT. PROD. REP. 20:79-110 (2003)). That list was then further prioritized by screening for optimal gRNA parameters including exclusion of TTTT PAM sequences, exclusion of gRNAs that were not predicted produce desirable hairpin structures, and gRNAs with high GC content (>50%) or long runs of T in the sequence. Editing efficiency of 36 potential gRNAs was tested in a transient protoplast assay by delivering ribonucleoprotein (RNP) complexes (that is synthesized gRNA (purchased from IDT) pre-complexed with purified LbCas12a protein) to maize leaf protoplasts using a poly-ethylene glycol transfection protocol. After RNP delivery, protoplast DNA was extracted and the target editing regions amplified from it via polymerase chain reaction (PCR). The products of that PCR reaction were subsequently analyzed for evidence of editing using both a T7E1-based enzymatic assay and next-gen (Illumina sequencing) to confirm the presence and frequency of edits at the target sites and gRNAs were ranked according to their efficiency. From these data, the six final gRNAs to go into vectors for stable transformation were selection (Table 9): two that targeted ZmFDF1, two that targeted ZmFDF2, and two that targeted both ZmFDF1 and ZmFDF2.

TABLE 9
Guide RNAs designed to target ZmFDF1 and ZmFDF2.

| Syngenta | | | |
|---|---|---|---|
| Construct | | | |
| ID | Editing Target | Guide RNA Name | Guide RNA Sequence |
| 27738 | ZmFDF1 | ZmFDF1\target37 | CTGACCAAGTTCTGCCTCA |
| (SEQ ID | | | GGAG (SEQ ID NO: 29) |
| NO: 17) | | ZmFDF1\target74 | CCATCAATTACCTTATCAG |
| | | | TCCC (SEQ ID NO: 30) |
| 27726 | ZmFDF2 | ZmFDF2\target72 | CGTAGTTCATCAAAGCCTT |
| (SEQ ID | | | CCTG (SEQ ID NO: 31) |
| NO: 16) | | ZmFDF2\target74 | TCAGTGCCAGGTAGGAACT |
| | | | CCTG (SEQ ID NO: 32) |
| | ZmFDF1 | ZmFDF2\target2 | CCTGAGAAGATTGAAAGCC |
| | ZmFDF2 | | GCAT (SEQ ID NO: 33) |
| 27769 | ZmFDF1 | ZmFDF2\target16 | TCCCTACCTGGTTCATGAG |
| (SEQ ID | ZmFDF2 | | AGTC (SEQ ID NO: 34) |
| NO: 18) | | | |

TABLE 10
Primers designed to amplify the edits
made with the guide RNAs above.

| Editing | Forward | Reverse | |
|---|---|---|---|
| Target(s) | Primer | Primer | Probe |
| ZmFDF1 | SEQ ID | SEQ ID | SEQ ID |
| | NO: 35 | NO: 36 | NO: 37 |
| ZmFDF1 | SEQ ID | SEQ ID | SEQ ID |
| | NO: 38 | NO: 39 | NO: 40 |
| ZmFDF2 | SEQ ID | SEQ ID | SEQ ID |
| | NO: 41 | NO: 42 | NO: 43 |
| ZmFDF2 | SEQ ID | SEQ ID | SEQ ID |
| | NO: 44 | NO: 45 | NO: 46 |
| ZmFDF1 and | SEQ ID | SEQ ID | SEQ ID |
| ZmFDF2 | NO: 47 | NO: 48 | NO: 49 |
| ZmFDF1 and | SEQ ID | SEQ ID | SEQ ID |
| ZmFDF2 | NO: 50 | NO: 51 | NO: 52 |

A series of three binary vectors that would generate loss of function alleles at ZmFDF1, ZmFDF2, or both ZmFDF1 and ZmFDF2 genome editing targets was designed by Syngenta between January and March 2022. To generate double stranded cuts in the targeted genes, a maize codon optimized version of the Cas12a enzyme derived from Lachnospiraceae bacterium was selected. This version of Cas12a contained a C965S amino acid substitution, an intron derived from the Arabidopsis thaliana BAF60 gene encoding a protein that belongs to the chromodomain remodeling complex, and a nuclear localization sequence derived from Simian Virus 40 at both the N-terminal and C-terminal ends of the amino acid sequence. Cas12a transcription was driven by a constitutive sugarcane ubiquitin 4 promoter derived from Saccharum officinarum. Transcription of Cas12a was terminated by a nopaline synthase terminator derived from Agrobacterium tumefaciens. In all vectors, guide RNA transcription was driven by an Oryza sativa U3 promoter for pol III dependent transcription of non-coding RNAs. For efficient guide RNA processing, all six guides were individually linked to trans-activating crRNA sequences derived from Streptococcus pyogenes to form a combined guide RNA and crRNA sequence.

For all 3 vectors, phosphomannose-isomerase (PMI) was chosen as the selectable marker for stable plant transformation. PMI transcription was driven by a ubiquitin promoter and transcription was terminated by a ubiquitin terminator, both derived from Zea mays. The Cas9 expression cassette, guide RNA expression and processing cassette, and PMI selectable marker cassette were linked to a standard binary vector containing left and right border elements derived from Agrobacterium tumefaciens and backbone elements. Backbone elements included a copy of the aminoglycoside 3′adenyltransferase gene that confers resistance to spectinomycin and streptomycin for maintenance of the vector in E. coli and Agrobacterium, derived from Escherichia coli. The backbone also included a bacterial expression cassette of two Agrobacterium tumefaciens genes driven by the VirG promoter, including the VirG transcriptional activator virulence factor and the RepA plasmid portioning protein. The backbone included the oVS1 origin of replication element derived from Pseudomonas sp. for plasmid replication in Agrobacterium tumefaciens and the ColE1 origin of replication element for plastic replication in Escherichia coli. All elements described above were assembled into three binary vectors in April 2022, validated by sequencing, and designated as Syngenta constructs 27726, 27738, and 27769.

Constructs 27726, 27738, and 27769 will be separately transformed into maize inbred line NP2222 via agrobacterium-mediated transformation. Transformants that were found to have the selectable marker stably integrated will be regenerated into plantlets and grown to maturity under greenhouse conditions. The presence of edits at the targets will be confirmed first via a Taqman assay designed to detect editing at the cut site for each gRNA, and a second assay deigned to detect the wild-type (uncut allele). In individual TO plants where the presence of a cut allele is detected by Taqman, a follow-up analysis was next generation sequencing (Illumina) to characterize the exact indels present and to select TO plants with the highest likelihood of chalcone synthase loss-of-function. In this analysis, any indel that resulted in a shift to the reading frame within an exon was interpreted as a loss-of-function allele.

### 6. Phenotyping Edited Maize Plants.

T0 maize plants with varying edits were observed for phenotypes associated with loss-of-function alleles sequenced in ZmFDF1 and ZmFDF2. In general, plant male reproductive phenotypes were associated with the total number of loss-of-function alleles to ZmFDF1 and ZmFDF2 out of the maximum possible total of four loss-of-function alleles. No abnormal phenotypes were observed in TO plants with one or two loss-of-function alleles. Interestingly, complete loss-of-function at one of the editing targets due to frameshift alleles in both copies of ZmFDF1 or ZmFDF2 did not result in a deviation from normal phenotype as long as both copies of the other respective gene did not have loss-of-function alleles. This suggests that ZmFDF1 and ZmFDF2 are functional duplicates. Several TO plants showed abnormal anthers that were reduced in size upon extrusion compared to most plants. These abnormally small anthers shed low quantities of pollen compared to most plants. Abnormal pollen shed was observed to be approximately 10 microliters of pollen per tassel per day, compared to the 500-1000 microliters of pollen per tassel per day that is typical of maize. This low quantity of pollen was nevertheless the standard yellow color normally observed in maize. TO plants with abnormal pollen and anthers were those plants with frameshift indels resulting in loss-of-function alleles to three of the four possible gene editing targets. TO plants with abnormal anthers that shed low quantities of pollen were manually self-pollinated and were self-fertile. No TO individuals with four loss-of-function alleles (frameshift indels to both copies of ZmFDF1 and both copies of ZmFDF2) were present in the TO generation. Common phenotypes associated with c2/whp1 maize (colorless aleurone, colorless silks, white pollen, premature cessation of pollen tube growth) were not observed in this population.

TABLE 11
FDF T0 maize plant anther and pollen phenotypes observed in
association with genotype. All loss-of-function alleles were
indels that caused a shift in reading frame in exon 2 of ZmFDF1
and/or ZmFDF2. No T0 individuals with four loss-of-function
alleles (frameshift indels to both copies of ZmFDF1 and both
copies of ZmFDF2) were present in the T0 generation.

| Number of Loss-of- | | |
|---|---|---|
| Function Alleles | Anther Phenotype | Pollen Phenotype |
| 0 | Normal | Normal |
| 1 | Normal | Normal |
| 2 | Normal | Normal |
| 3 | Reduced in size | Reduced pollen shed |
| 4 | ND | ND |

ND = No Data

T1 seed obtained by self-pollinating TO plants was planted to further characterize the association between loss-of-function genotypes and plant reproductive phenotypes. Genotyping selected progeny that were homozygous for loss-of-function alleles to ZmFDF1 or ZmFDF2, progeny with complete loss-of-function to either ZmFDF1 or ZmFDF2 and one functional copy of the other respective gene, and progeny that were homozygous for loss-of-function alleles to both ZmFDF1 and ZmFDF2. As part of pollen viability screening, T1 plants were self-pollinated to produce T2 seed. T2 seed was also planted in a greenhouse and the same panel of genotypes was selected for phenotyping. A similar association between edited allele genotype and plant phenotype was observed across T1 and T2 plants screened. Complete loss-of-function at one of the editing targets due to frameshift alleles in both copies of ZmFDF1 or ZmFDF2 did not result in a deviation from normal phenotype as long as both copies of the other respective gene did not have loss-of-function editing alleles. T1 and T2 plants with three of four possible showing loss-of-function frameshift alleles continued to show abnormal pollen and anther phenotypes. In these individuals, anthers were universally smaller than is standard for the line and these smaller anthers shed lower quantities of pollen. Abnormal pollen shed was observed to range between approximately 10 microliters of pollen per tassel per day to 100 microliters of pollen per tassel per day, compared to the 500-1000 microliters of pollen per tassel per day that is typical of maize. Across the entire range of pollen volume shed per day, pollen was the standard yellow color observed in maize. Pollen samples were self-fertile in all T1 and T2 plants tested.

T1 and T2 plants with homozygous frameshift indels resulting in complete loss-of-function to both ZmFDF1 and ZmFDF2 were observed to show a range of phenotypes. Select progeny showed a similar reduced anther size, reduced pollen shed phenotype as siblings with one intact copy of ZmFDF1 or ZmFDF2. Pollen from these progenies was self-fertile. Other sibling progeny demonstrated a loss of normal anther development in which anthers did not fully mature, failed to extrude from florets, and did not contain any viable pollen grains. These individuals were all functionally male-sterile due to a complete absence of pollen. Further selection for specific indels in ZmFDF1 and ZmFDF2 is required to demonstrate whether the complete loss-of-function phenotype is characterized by a spectrum in anther and pollen phenotypes or is a consistent antherless, pollenless phenotype. Select indels that result in a frameshift to the coding sequence may cause a complete loss-of-function to the gene target, while other indels that result in a frameshift may not completely eliminate function based on the exact position in the target coding sequence. Further phenotyping may demonstrate that the spectrum in anther and pollen phenotypes is influenced by environmental factors independent of the specific indels in ZmFDF1 and ZmFDF2.

It is hypothesized that application of exogenous flavonoids to the developing anthers of antherless, pollenless genotypes will restore male fertility. Male fertility restoration treatments may be applied to developing tassels by delivering the treatment down the leaf whorl, injecting the treatment through the leaf whorl into the air pocket surrounding the developing tassel, or directly to the developing tassel as it emerges from the leaf whorl. Male fertility restoration treatments may also be applied as a foliar treatment to the leaf whorl, the entire leaf canopy, or as a drench. Treatments may consist of quercetin exogenously applied at one or multiple microspore development stages. Quercetin may be applied exogenously as a dry powder, dissolved in a solvent or solution, or suspended in a formulation. Liquid solutions and suspensions of quercetin dihydrate may include inert ingredients that enhance the penetrance of quercetin into plant tissues.

TABLE 12
FDF T1 and T2 maize plant anther and pollen phenotypes
observed in association with genotype. All loss-of-
function alleles were indels that caused a shift in
reading frame in exon 2 of ZmFDF1 and/or ZmFDF2.

| Number of | | |
|---|---|---|
| Frameshift | | |
| Alleles | Anther Phenotype | Pollen Phenotype |
| 1 | Normal | Normal |
| 2 | Normal | Normal |
| 3 | Reduced in size | Reduced pollen shed |
| 4 | Ranged from in | Ranged from reduced |
| | reduced size | pollen shed to absence of |
| | to undeveloped | pollen in undeveloped anthers |

### 7. Construct Information.

TABLE 13
Components found in construct 25206 (SEQ ID NO: 12).

| Name | Type | Minimum | Maximum | Length |
|---|---|---|---|---|
| cRepA-03 | CDS | 17242 | 18315 | 1074 |
| cVirG-01 | CDS | 16487 | 17212 | 726 |
| prVirG-01 | promoter | 16282 | 16412 | 131 |
| cSpec-03 | CDS | 15399 | 16187 | 789 |
| xTAG-02 | misc_feature | 14942 | 14981 | 40 |
| xSTOPS-01 | misc_feature | 14930 | 14941 | 12 |
| tUbi1-01 | terminator | 13852 | 14886 | 1035 |
| cPMI-12 | CDS | 12669 | 13847 | 1179 |
| iUbi1-02-01 | intron | 11648 | 12657 | 1010 |
| TSS | misc_feature | 11565 | 11565 | 1 |
| prUbi1-10 | promoter | 10665 | 12657 | 1993 |
| xSTOPS-01 | misc_feature | 10653 | 10664 | 12 |
| tNOS-05-01 | terminator | 10374 | 10626 | 253 |
| rHDV-01 | misc_RNA | 10306 | 10373 | 68 |
| rCrRNA-10 | misc_RNA | 10284 | 10305 | 22 |
| rCrRNATaCHSG-02 | misc_RNA | 10264 | 10305 | 42 |
| TaCHSGTarget2 | misc_feature | 10264 | 10283 | 20 |
| rHH-05 | misc_RNA | 10227 | 10263 | 37 |
| rHHTaCHSG-02 | misc_feature | 10221 | 10263 | 43 |
| pairing\sequence | misc_feature | 10221 | 10226 | 6 |
| rHDV-01 | misc_RNA | 10146 | 10213 | 68 |
| rCrRNA-10 | misc_RNA | 10124 | 10145 | 22 |
| rCrRNATaCHSG-01 | misc_RNA | 10104 | 10145 | 42 |
| TaCHSGTarget1 | misc_feature | 10104 | 10123 | 20 |
| rHH-05 | misc_RNA | 10067 | 10103 | 37 |
| rHHTaCHSG-01 | misc_feature | 10061 | 10103 | 43 |
| pairing\sequence | misc_feature | 10061 | 10066 | 6 |
| rHDV-01 | misc_RNA | 9977 | 10044 | 68 |
| rTracrRNA-11 | misc_RNA | 9904 | 9976 | 73 |
| rHH-03 | misc_RNA | 9861 | 9903 | 43 |
| TracrRNA\paring\sequence | misc_feature | 9861 | 9866 | 6 |
| rHDV-01 | misc_RNA | 9786 | 9853 | 68 |
| rTracrRNA-11 | misc_RNA | 9713 | 9785 | 73 |
| rHH-03 | misc_RNA | 9670 | 9712 | 43 |
| TracrRNA\paring\sequence | misc_feature | 9670 | 9675 | 6 |
| iSoUbi4-02 | intron | 8299 | 9656 | 1358 |
| u5SoUbi4-02 | 5′UTR | 8234 | 8298 | 65 |
| prSoUbi4-02 | promoter | 7855 | 9656 | 1802 |
| xSTOPS-01 | misc_feature | 7836 | 7847 | 12 |
| tZmMTL-01 | terminator | 6834 | 7835 | 1002 |
| NLS | sig_peptide | 6762 | 6824 | 63 |
| I\to\V\mutation | misc_feature | 6192 | 6194 | 3 |
| L\to\V\mutation | misc_feature | 6147 | 6149 | 3 |
| +5C | misc_feature | 2611 | 2611 | 1 |
| +4G | misc_feature | 2610 | 2610 | 1 |
| NLS | transit_peptide | 2607 | 2657 | 51 |
| cCas9-11 | CDS | 2607 | 6827 | 4221 |
| iSoUbi4-02 | intron | 1235 | 2592 | 1358 |
| u5SoUbi4-02 | 5′UTR | 1170 | 1234 | 65 |
| prSoUbi4-02 | promoter | 791 | 2592 | 1802 |
| e35S-11 | enhancer | 492 | 784 | 293 |
| eFMV-06 | enhancer | 292 | 485 | 194 |
| eNOS-01 | enhancer | 168 | 259 | 92 |

TABLE 14
Components found in construct 27726 (SEQ ID NO: 16).

| Name | Type | Minimum | Maximum | Length |
|---|---|---|---|---|
| cRepA-05 | CDS | 14034 | 15107 | 1074 |
| cVirG-06 | CDS | 13279 | 14004 | 726 |
| prVirG-02 | promoter | 13074 | 13204 | 131 |
| cSpec-03 | CDS | 12191 | 12979 | 789 |
| xTAG-02 | misc_feature | 11734 | 11773 | 40 |
| xSTOPS-01 | misc_feature | 11722 | 11733 | 12 |
| tUbi1-06 | terminator | 10665 | 11699 | 1035 |
| cPMI-14 | CDS | 9477 | 10652 | 1176 |
| iUbi1-07 | intron | 8460 | 9469 | 1010 |
| TSS | misc_feature | 8377 | 8377 | 1 |
| prUbi1-18 | promoter | 7477 | 9469 | 1993 |
| ZmFDF2\target74 | misc_feature | 7439 | 7461 | 23 |
| rLbCrRNA-01 | misc_RNA | 7418 | 7438 | 21 |
| rLbgRNACas12aZmFDF2.74-01 | misc_feature | 7418 | 7461 | 44 |
| prOsU3-01 | promoter | 7042 | 7416 | 375 |
| ZmFDF2\target72 | misc_feature | 7004 | 7026 | 23 |
| rLbCrRNA-01 | misc_RNA | 6983 | 7003 | 21 |
| rLbgRNACas12aZmFDF2.72-01 | misc_feature | 6983 | 7026 | 44 |
| prOsU3-01 | promoter | 6607 | 6981 | 375 |
| xSTOPS-05 | misc_feature | 6590 | 6601 | 12 |
| tNOS-05-01 | terminator | 6327 | 6579 | 253 |
| xSGGSlinker-02 | misc_feature | 6283 | 6294 | 12 |
| xSGGSlinker-02 | misc_feature | 6271 | 6282 | 12 |
| iAtBAF60-01 | intron | 2672 | 3080 | 409 |
| Real ATG | misc_feature | 2151 | 2153 | 3 |
| xLinker-06 | misc_feature | 2061 | 2150 | 90 |
| cLbCas12a-40 | CDS | 2037 | 6318 | 4282 |
| iSoUbi4-02 | intron | 661 | 2018 | 1358 |
| u5SoUbi4-02 | 5′UTR | 596 | 660 | 65 |
| prSoUbi4-02 | promoter | 217 | 2018 | 1802 |
| xSTOPS-01 | misc_feature | 184 | 195 | 12 |
| xTAG-06 | misc_feature | 144 | 183 | 40 |

TABLE 15
Components found in construct 27738 (SEQ ID NO: 17).

| Name | Type | Minimum | Maximum | Length |
|---|---|---|---|---|
| cRepA-05 | CDS | 14034 | 15107 | 1074 |
| cVirG-06 | CDS | 13279 | 14004 | 726 |
| prVirG-02 | promoter | 13074 | 13204 | 131 |
| cSpec-03 | CDS | 12191 | 12979 | 789 |
| xTAG-02 | misc_feature | 11734 | 11773 | 40 |
| xSTOPS-01 | misc_feature | 11722 | 11733 | 12 |
| tUbi1-06 | terminator | 10665 | 11699 | 1035 |
| cPMI-14 | CDS | 9477 | 10652 | 1176 |
| iUbi1-07 | intron | 8460 | 9469 | 1010 |
| TSS | misc_feature | 8377 | 8377 | 1 |
| prUbi1-18 | promoter | 7477 | 9469 | 1993 |
| ZmFDF1\target72 | misc_feature | 7439 | 7461 | 23 |
| rLbCrRNA-01 | misc_RNA | 7418 | 7438 | 21 |
| rLbgRNACas12aZmFDF1.72-01 | misc_feature | 7418 | 7461 | 44 |
| prOsU3-01 | promoter | 7042 | 7416 | 375 |
| ZmFDF1\target37 | misc_feature | 7004 | 7026 | 23 |
| rLbCrRNA-01 | misc_RNA | 6983 | 7003 | 21 |
| rLbgRNACas12aZmFDF1.37-01 | misc_feature | 6983 | 7026 | 44 |
| prOsU3-01 | promoter | 6607 | 6981 | 375 |
| xSTOPS-05 | misc_feature | 6590 | 6601 | 12 |
| tNOS-05-01 | terminator | 6327 | 6579 | 253 |
| xSGGSlinker-02 | misc_feature | 6283 | 6294 | 12 |
| xSGGSlinker-02 | misc_feature | 6271 | 6282 | 12 |
| iAtBAF60-01 | intron | 2672 | 3080 | 409 |
| Real ATG | misc_feature | 2151 | 2153 | 3 |
| xLinker-06 | misc_feature | 2061 | 2150 | 90 |
| cLbCas12a-40 | CDS | 2037 | 6318 | 4282 |
| iSoUbi4-02 | intron | 661 | 2018 | 1358 |
| u5SoUbi4-02 | 5′UTR | 596 | 660 | 65 |
| prSoUbi4-02 | promoter | 217 | 2018 | 1802 |
| xSTOPS-01 | misc_feature | 184 | 195 | 12 |
| xTAG-06 | misc_feature | 144 | 183 | 40 |

TABLE 16
Components found in construct 27769 (SEQ ID NO: 18).

| Name | Type | Minimum | Maximum | Length |
|---|---|---|---|---|
| cRepA-05 | CDS | 14034 | 15107 | 1074 |
| cVirG-06 | CDS | 13279 | 14004 | 726 |
| prVirG-02 | promoter | 13074 | 13204 | 131 |
| cSpec-03 | CDS | 12191 | 12979 | 789 |
| xTAG-02 | misc_feature | 11734 | 11773 | 40 |
| xSTOPS-01 | misc_feature | 11722 | 11733 | 12 |
| tUbi1-06 | terminator | 10665 | 11699 | 1035 |
| cPMI-14 | CDS | 9477 | 10652 | 1176 |
| iUbi1-07 | intron | 8460 | 9469 | 1010 |
| TSS | misc_feature | 8377 | 8377 | 1 |
| prUbi1-18 | promoter | 7477 | 9469 | 1993 |
| ZmFDF2\target16 | misc_feature | 7439 | 7461 | 23 |
| rLbCrRNA-01 | misc_RNA | 7418 | 7438 | 21 |
| rLbgRNACas12aZmFDF2.16-01 | misc_feature | 7418 | 7461 | 44 |
| prOsU3-01 | promoter | 7042 | 7416 | 375 |
| ZmFDF2\target2 | misc_feature | 7004 | 7026 | 23 |
| rLbCrRNA-01 | misc_RNA | 6983 | 7003 | 21 |
| rLbgRNACas12aZmFDF2.2-01 | misc_feature | 6983 | 7026 | 44 |
| prOsU3-01 | promoter | 6607 | 6981 | 375 |
| xSTOPS-05 | misc_feature | 6590 | 6601 | 12 |
| tNOS-05-01 | terminator | 6327 | 6579 | 253 |
| xSGGSlinker-02 | misc_feature | 6283 | 6294 | 12 |
| xSGGSlinker-02 | misc_feature | 6271 | 6282 | 12 |
| iAtBAF60-01 | intron | 2672 | 3080 | 409 |
| Real ATG | misc_feature | 2151 | 2153 | 3 |
| xLinker-06 | misc_feature | 2061 | 2150 | 90 |
| cLbCas12a-40 | CDS | 2037 | 6318 | 4282 |
| iSoUbi4-02 | intron | 661 | 2018 | 1358 |
| u5SoUbi4-02 | 5′UTR | 596 | 660 | 65 |
| prSoUbi4-02 | promoter | 217 | 2018 | 1802 |
| xSTOPS-01 | misc_feature | 184 | 195 | 12 |
| xTAG-06 | misc_feature | 144 | 183 | 40 |

What is claimed is:

1. A method of seed production, comprising:
a. obtaining at least one first plant, wherein the first plant is a flavonoid-dependent fertile plant comprising a mutation in an flavonoid-dependent fertility (“FDF”) gene;
b. obtaining at least one second plant, wherein the second plant is male fertile;
c. allowing pollination by the at least one second plant to the at least one first plant; and
d. obtaining progeny seed thereof.

2. The method of claim 1, wherein the first plant and the second plant are monocot plants selected from the group consisting of wheat, maize, and rice.

3. (canceled)

4. A flavonoid-dependent fertile (“FDF”) plant comprising a mutation in an FDF gene, wherein the mutation is a homozygous knock-out mutation.

5. (canceled)

6. (canceled)

7. The FDF plant of claim 2, wherein the FDF plant is a wheat plant plant and the FDF gene is selected from TaFDF1A (SEQ ID NO: 1), TaFDF1B (SEQ ID NO: 2), TaFDF1D (SEQ ID NO: 3), and a combination thereof.

8. (canceled)

9. The FDF plant of claim 2, wherein the FDF plant is a maize plant, and the FDF gene is ZmFDF1 (SEQ ID NO: 4), ZmFDF2 (SEQ ID NO: 5), and a combination thereof.

10. (canceled)

11. The FDF plant of claim 9, wherein the FDF plant further comprises a mutation in C2 (SEQ ID NO: 26) and/or WHP (SEQ ID NO: 28).

12. The FDF plant of claim 2, wherein the FDF plant is a rice plant, and the FDF gene is OsFDF (SEQ ID NO: 57).

13. (canceled)

14. A method of propagating an FDF plant, comprising:
a. obtaining at least one plant, wherein the plant is a flavonoid-dependent fertile (“FDF”) plant comprising a mutation in an FDF gene;
b. applying a composition comprising a flavonoid to the plants of step a.;
c. allowing self-pollination to occur; and
d. obtaining progeny seed thereof.

15. The method of claim 14, wherein the flavonoid is quercetin and the composition comprising quercetin is a liquid solution or a powder.

16. (canceled)

17. The method of claim 15, wherein the liquid solution comprises quercetin mixed in water, propylene glycol, or other solvent or solution, wherein the quercetin is suspended in a solvent or solution at a concentration of 1 mg/L to 100 mg/L, 2.5 mg/L to 50 mg/L, or approximately 5 mg/L.

18. (canceled)

19. The method of claim 17, wherein the liquid solution comprising quercetin is saturated.

20. (canceled)

21. The method of claim 20, wherein the powder comprises a mixture of quercetin and a carrier compound at a ratio between 1000:1 to 1:1000, wherein the carrier compound is selected from the group consisting of crystalline silica, talc, metallic powder, and mica minerals.

22. (canceled)

23. (canceled)

24. The method of claim 14, wherein the composition comprising a flavonoid is applied to female organs of a flower on the at least one plant designated as a female plant and is applied by a method selected from the group consisting of painting, misting, spraying, and root drenching.

25. (canceled)

26. The method of claim 14, wherein the FDF gene is a wheat gene selected from the group consisting of SEQ ID NOs: 1-3 and a combination thereof, or wherein the FDF gene is a maize gene selected from the group consisting of SEQ ID NOs: 4-5 and a combination thereof, or wherein the FDF gene is a rice gene comprising SEQ ID NO: 57.

27. (canceled)

28. (canceled)

29. A plant made by the method of claim 14.

30. A method of hybrid seed production, comprising:
a. obtaining at least one inbred female FDF plant, wherein the inbred female FDF plant comprises a mutation in an FDF gene;
b. obtaining pollen from at least one inbred male plant, wherein the inbred male plant produces fertile pollen;
c. pollinating the inbred female FDF plant with the fertile pollen from the inbred male plant; and
d. obtaining hybrid progeny thereof.

31. The method of claim 30, wherein the inbred female FDF plant is a maize plant, a wheat plant, or a rice plant, wherein the FDF wheat plant comprises a mutation in a gene selected from the group consisting of SEQ ID NOs: 1-3, and a combination thereof, wherein the FDF maize plant comprises a mutation in a gene selected from the group consisting of SEQ ID NOs: 4-5 and a combination thereof, or wherein the FDF rice plant comprises a mutation in SEQ ID NO: 57.

32. (canceled)

33. (canceled)

34. (canceled)

35. A plant made by the method of claim 30.

36. A method of restoring fertility to an FDF plant comprising a mutation in an FDF gene, the method comprising applying a composition comprising a flavonoid to the FDF plant.

37. The method of claim 36, wherein the FDF plant is a maize plant, a wheat plant, or a rice plant, wherein the FDF maize plant comprises a mutation in a gene selected from the group consisting of SEQ ID NOs: 4-5 and a combination thereof, wherein the FDF wheat plant comprises a mutation in a gene selected from the group consisting of SEQ ID NOs: 1-3, and a combination thereof, or wherein the FDF rice plant comprises a mutation in SEQ ID NO: 57.

38. (canceled)

39. (canceled)

40. The method of claim 37, wherein the FDF maize plant further comprises a mutation in C2 (SEQ ID NO: 26) and/or WHP (SEQ ID NO: 28).

41. (canceled)

42. The method of claim 36, wherein the flavonoid comprises quercetin mixed in water, propylene glycol, or other solvent or solution.

43. (canceled)

44. The method of claim 36, wherein the composition comprising a flavonoid further comprises a carrier compound, wherein the carrier compound is selected from the group consisting of crystalline silica, talc, metallic powder, and mica minerals.

45. (canceled)

46. The method of claim 36, wherein the composition comprising a flavonoid is applied to anthers, silks, stigmas, florets, spikes, leaf whorls, leaf canopy, or roots by a method selected from the group consisting of painting, misting, spraying, and root drenching.

47. (canceled)


SEQUENCE LISTING (57 sequences)

SEQ ID NO: 1 - DNA, 1999 bp, Triticum aestivum
  source 1..1999: mol_type = genomic DNA
  exon 1..199
  intron 200..1025
  exon 1026..1999
atgtctggccttggtggagtcgggagcattggcggaggcggcaacggcagaagccaaggcttggggggcaaggccaagctgctcgcgctcggcaagggactccctgaacaagttctaccccaggagaagctcgtggagacctacctccaggacaccagctgcgacgatcccgccaccagggccaagctcgaacgcctctgtgagttcccttccttcatgtcatttgtagttttacatataacagcttcccgggctgagagttttaagttggcaacaatagttgttctctgtcgacttcgaaaacaaaaatagttgattctccgtaaagagatcattgacactagtgagttgtagtaatactgccatttggttgcagcatcttagtttacagaatctaccatgctttagtataactgtcagttatcataggtgcaaagtgcgacaaatctccactcggtaaataaaatgttaacatggtcattattactaaatcccagttacagtttagcttaagaaaatcagcagtacctaacctattttatagagtatataaattgtgtaaaatggaattagtgtgatgagaaacacaacaaaattccacctagaaacaaaattccacaagaaaagactgtaggaaaactcgcctacagtcattgaccgctcaaaaaaaaatccacctagaaacaacatgcatgcacaactagaattattactctccaattgtacttattttttgctctaggagaaacactaggacacccctcaaaaaagaacactagaacaccatttgtttttgtccttctgtttgtggtgagggctaagcaaataagagttaatagtacataatcacaaactataaactatacttatttgttatgtattccctccgtttggaaataagtgacgtggttttagttcaaatttgaactaaacccacaacacttatttccgaattgaggaagtagtaatttgcatcagtccctccaatgtatgtataatggttcccctgtgatttgatttgacaggcaagaccacaacagtgaggacaaggtacactgtcatgtcgaaggagctcctggatgaatacccagaattgaagacagaaggctctccaacattgacgccacggcttgacatctgcaatgccgcggtgcttgaacttggtgctgctgcagctcgtgccgcccttggtgagtggggccgtccagcagctgatattacccacctggtctacatctcctccagcgagcttcgcctcccagggggtgatcttttcctggcaacccgtcttggcctctctccaaacacagtgcgcacatcccttctcttccttggctgctcaggtggtgctgccgccctccgcactgccaaggacattgcagagaacaacccagggagccgtgtccttgtaatagcggcggagacaactgtgttgggcttccggccgccaagccctgatcgtccttatgaccttgttggtgctgctctgtttggtgatggtgcatcagctgcaattattggagcaagccctatcaaagcagaagaggatcctttcttggagcttgagttctcaacccaggagttcctaccagggacagacaaggtaattgatggcaaaatctcagaggaagggattaacttcaaactggggcgtgatttgcctgaaaagattgaaagccgcatagaaggcttctgcaggatactcatggacaaggttgggataaaggagttcaatgatgtattttgggctgtgcatcccggtggaccagcaatattgaacaggctggaggtttgtcttcaacttgagccagataaactcaagatcagtagaaaggccctgatgaactatgggaatgtgagcagtaacacagtcttctatgtgctggagtatttgagggatgaattgaagaaaggggcaataagggaagagtggggattgatcttggcttttggcccaggcatcacatttgaaggactgctagttcggggcattaactga

SEQ ID NO: 2 - DNA, 1934 bp, Triticum aestivum
  source 1..1934: mol_type = genomic DNA; note = reverse orientation
  exon 1..974: note = reverse orientation
  intron 975..1735: note = reverse orientation
  exon 1736..1934: note = reverse orientation
tcagttagtgccccgaactagcattccttcaaatgtgatgcctgggccaaaagccaagatcaatccccactcttcccttattgcccctttcttcaattcatccctcaaatactccagcacatagaagactgtgttgctgctcacattcccatagttcatcagggcctttctactgatcttgagtttacctggctcaagttcaagacaaacctccagcctgttcaatattgctggtccaccaggatgcacagcccaaaatacatcattgaattcctttatcccaaccttgtccatgagtatcctgcagaagccttctatgcggctttcaatcttttcaggcaaatcacgccccagtttgaagttaatcccttcctctgagatcttgccatcaattaccttgtctgtccctggtaggaactcctgggttgagaactcaagctccaagaaaggatcctcttctgctttgatagggcttgctccaataattgcagctgatgcaccatcaccaaacagagcagcaccaacaaggtcataaggacgatcagggcttggcggccggaagcccaacacagttgtctccgccgctattacaaggacacggctccctgggttgttctctgcaatgtccttggcagtgcggagggcggcagcaccacctgagcagccaaggaagagaagggatgtgcgcactgtgtttggagagaggccaaggcgggttgccaggaaaagatcaccccccgggaggcgaagctcgctggaggagatgtagaccaggtgggtaatatcagctgctggacggccccactcaccaagggcggcacgagctgcagcagcaccaagttcaagcaccgcggcattgcagatgtcaagccgtggtgtcaatgttggagtgccttctgtcttcaattctgggtgttcatccaggagctccttcgacatgacagtgtaccttgtcctcactgttgtggtcttgcctgtcaaatcaaatcacaggggaaccattatacatacattggagggactgatgcaaattactactccctccattcggaaataagtgacgtggttttagttcaaataacaaataagtatagtttatagtttgtgattatgtactattaactcttatttgcttagccctcaccacaaacagaaggaggaaaataaatggtgttctagtgttcttatttgagggggtgtttctcctagagcaaaaaataattacaattggagagtaataattctagttgtgcatgcatgttgtttctaggtggatttttttttgagcggtgaatgaccgtaggagagttctcctagtcttttcttgtggaattttgtttctaggtggcattttgttgtgtttctcatcacactaattccattttacacatgatttatactgtaaaatagtttagatactgctgattttcttaacctaaactgtgaccgggatttagtactaatgaccatgtgaacattttatttatttactgagtggagattgattgtctttcccattgtactaattgcactttgcacctatgataactgatagttaaactaaagcatggtagattctgtacactaagatgttacaaccagatggcaaccttattacaactcactgttgccagtgatctctttaaggagagccaactattgttgccaacttaaatgcctcagcccgagaagcaggtatgtaaaactgcagatgacatgaaggaagggaactcacagaggcgctcgagcttggccctggtggcgggatcgtcgcagctggtgtcctggaggtaggtctcgacgagcttctcctggggtagaacttgctcagggagacccttgccgagcgcgagcagcttggccttgccccccgagccatggcttctgccgttgctgcccccgccgacgctcccgagtccaccaaggccagacat

SEQ ID NO: 3 - DNA, 1975 bp, Triticum aestivum
  source 1..1975: mol_type = genomic DNA
  exon 1..199
  intron 200..1001
  exon 1002..1975
atgtctggccttggtggactcgggagcatcggcgggggcggcaacggcataagccacggctcggggggcaaggccaagctgctcgcactcggcaagggtctccctgaacaagttctaccccaggagaagctcgtcgagacctacctccaggacaccagctgcgacgatcccgccaccagggccaagctcgaacgcctctgtgagcttccttccttcatgtcatttgtagttttacatataactgcttctcgggctgagagttttaagttggcagcaatagttgttctccgtcgacttcgaaaacaaaaatagttgattctccgtaaagagatcattgccatttggttgtagcatcttagtttacagaatctaccatgctttagtataactgtcagttatcataggtgcaaagtgcaacgaatctccactcggtaaataaaatgttcacatggtcattattactaaatcccagttacagtttagctcaagaaaatcagcagtacctaacctattttagagtatgtaaatcatgtgtaaaatggaattagtgtgatgagaaacacaacaaaattctacctagaaacaaaatttcacaagaaaagagactgtaggaaaactctcctacagtcattgaccgctaaaaaaaaaaatccacctagaaacaacatgcgtgcgcaactagaattattactctccaattgtacttattttttgctctaggacaaacactagaccccctcaaaaaagaacactagaacaccatttgtttttgtccttctatttgtggtgagggctaagcaaacaagagttaatagtacataatcacaaactataaactatacttatttgttatgtattccctccgttcggaaataagtgacatggttttagttcaaatttgaactaaactcacaacacttatttccgaattgaggaagtagtaatttgcatcagtccctccaatgtatgtataattgttcccctgtgatttgatttgacaggcaagaccacaacagtgaggacaaggtacactgtcatgtcgaaggagctcctggatgaatacccagaattgaagacagaaggctctccaacattgacgccacggcttgacatctgcaatgccgcggtgcttgaacttggtgctgctgcagctcgtgccgcccttggtgagtggggccgtccagcagctgatattacccacctggtctacatctcctccagcgagcttcgcctcccagggggtgatcttttcctggcaacccgtcttggcctctctccaaacacagtgcgcacatcccttctcttccttggctgctcaggtggtgctgccgccctccgcactgccaaggacattgcagagaacaacccagggagccgtgtccttgtaatagcggcggagacaactgtgttgggcttccggccgccaagccctgatcgtccttatgacctcgttggtgctgctctgtttggtgatggtgcatcagctgcaattattggagcaagccctatcaaagcagaagaggatcctttcttggagcttgagttctcaacccaggagttcctaccagggacagacaaggtaattgatggcaagatctcagaggaagggattaacttcaaactggggcgtgatttgcctgaaaagattgaaagccgcatagaaggcttctgcaggatactcatggacaaggttgggataaaggagttcaatgacgtattttgggctgtgcatcccggcggaccagcaatattgaacaggctggaggtttgtcttgaacttgagccagataaactcaagatcagtagaaaggccctgatgaactatgggaatgtgagcagcaacacagtcttctatgtgctggagtatttgagggatgaattgaagaaaggggcaataagggaagagtggggattgatcttggcttttggcccaggcatcacatttgaaggaatgctagttcggggcattaactga

SEQ ID NO: 4 - DNA, 1785 bp, Zea mays
  source 1..1785: mol_type = genomic DNA; note = reverse orientation
  3'UTR 1..1172: note = reverse orientation
  exon 1173..1253: note = reverse orientation
  intron 1254..1502: note = reverse orientation
  exon 1503..1686: note = reverse orientation
  5'UTR 1687..1785: note = reverse orientation
acaagcagtattaaagtaatcctgcaatcattgcatagtacagtggaacaacactacaacaggctgtatagtgccttatttctacaatgtctaggaagcaaactcaagtaccttacactgtttgatagatgaagaacacaaatacagtaaaatattagcgatgtgctatgtggtgctcagcttccaccgagacatctatgctctttatgtgatgtttctgtatatataattaatctttatgcttgccatgtacccaccattatattagcttgtgaaggttttctttacacctcgaactagcagtccttcaaaggtgatgcctgggccaaaagccaagatcaatccccactcttcacttattgcccctttctttaactcgtccctcaagtactccagcacatagaagattgtattgctgctcacatttccatagttcattaaggcttttctgctaatcttgagcttctctggctgaagtttaaggcagaactcaagcctgttcaatattgctggtccaccaggatgcacagcccaaaatatatcgttgaaatcctttatccctaccttgttcatgagagtcctgcagaaaccttctatgcggctttcaatcttctcaggcaaatcacgccctagtttaaaattaattccttcctctgagattttgccatcaattaccttatcagtcccaggtaggaactcctgcgtggagaactcaagctccaagaaaggattttctgctggtgtcatgggacctgctcctataatcacggctgatgcaccatcaccaaacagcgcagcaccaacaaggtcataaggacggtcataacttggtggccggaagcctagcacagtggtctcagcagctgttactaggacacggctccctggattgttctcagcaatgtccttggcagtgcggagggcagcagcaccaccggagcagccaaggaagagaagggaagtgcgcacggtgtttgggctaaggccaaggcgagcagccaggtgaaggtcgccccctgggagacgaaggtcacttgatgagatgtagatgaggtgggtaatatcagctgcagggcgaccccattcgtcaagggcagcgcgagctgcggcagcaccaaggtcgatcaccgcggcattgcagatgtcaaggcggggtgtcagtgtcggagtaccctccatcttcagctctgggtgcttatccaatagctccttggacataacagtgtaccttgtcttcactgttgtagtcgtgcctggaaaattaaatcatttgggcaatactcatctgtttatcggaggcaaatcgaatcatcacataaatactgaaggctgtatatggtgatctattgtactcattgtcctttccttattcagtgagcgttgttgtttagaccatagatgtttatgactagagctaagctgcatttgtcgacctaaaattttcaacgtgaagacaagatgaatttatagaacaacaactgtgatgaaacaaagaagcttacaaaggcgctgaagcttggccctggtggcgggatcgtcgcagctgctttcttggaggtagctctccacaaccttctcctgaggcagaacttggtcaggaaagctcttgcccaaggcgagcagcattgccttggccctggagctctggctcttgccattggcgcccgtgcttccactgccggtcattgtcgccgcactggcgttccttgatctactacctttctgtttctggtgctgtactgctgtgattttcttgtgcggcgaggccacggcaacatctttggt

SEQ ID NO: 5 - DNA, 3001 bp, Zea mays
  source 1..3001: mol_type = genomic DNA; note = reverse orientation
  3'UTR 1..263: note = reverse orientation
  exon 246..1252: note = reverse orientation
  intron 1253..1354: note = reverse orientation
  exon 1355..1631: note = reverse orientation
  5'UTR 1631..1639: note = reverse orientation
gaggggtttgagaagcttttgaatgagatcagcttgaacaagccggatacagtggttctccaagaaaggcgagacagccaagggggaggagctgcttgatgtgatggaatcaaaggaggtagccccgaattaccttacctacaatgctatcatgcaggggtattgcaaggaggggaatgtcggcgcagcgatgcgtgtgttcaagaagatgaaggtgatgaagaggcgggaggggagaagtgacttgggtgtgtctgctcattcgcagatatatgtggtgctgtttaggagtttggtggagaaacagaagtttggtgatgcgctgtggatttttaagagctgctttgcaatgaaggcagcaccaccatttgaggctgttaaaggtttggtagagggtttggtgaaggcaggcaggtcagcagaggccaaggatgttgttgccaagatggcctttcttgtgaaaggcgatgctaaacttgcttgggagaaggttgttggcgaatttttttttcaaagagggaacaacaaatttgaatccatgatttgctaggctttcatgcattttggacttggaatgatgatggcaaagaagataggctttcaacatgggatccaaagttgccttcttgttgtcactggagtttattttgcaaggtccgtggttagatcaggatgtaatggatgctccctttactgtaagaacttgctgaattttgtcttttaataattttgaaatgatgttaaagtttatatttgttgaagtgcttaggagatcctgatacatgatacatatactatctatctatctatctatcatcttaacatgtttgttgtaatgtgtgtttgatgcctcatcacataattgctctgcttttcctttgaaaatatgatagctgcgatgtaatcagttgactcatgcataaatctgtattgaagtaaatcctgcaattagtgcatagtacagtggaacaacaggctgtataatgccttatttctgcaatgtttttcttggaggcaaactcaactacttacattgtttgatagataaacacataaatacagtaaaatgttagcgatgtactacgcggtgcacagctttcaccaatgcatctatgctctttatcctatgtttctgtatatatgattaatctttatgcttgctatgcaaaccttccctctgttatattagcttgtgaatgttttcttaacaccttggactagaagtccttcgaaggtgatgcctgggccaaaagccaagatcaatccccagtcttcacttattgtccctttctttaactcgtccctcaagtactccagcacatagaagattgtattgctgctcacatttccgtagttcatcaaagccttcctgctaatcttgagcttctctggctgaagttgaaggcagatctcaagcctgtttagtattgctggtccgccaggatgcacagcccaaaacacatcgttgaaatcctttatccctacctggttcatgagagtcctgcagaacccttctatgcggctttcaatcttctcaggcaaatcacgccccagtttaaaattaattccttcctctgagatgttgccatcgatcactttatcagtgccaggtaggaactcctgtgtggagaactgaagctccaagaaaggattttcagctggtgccatggggcctgctcctataatcacagctgaggctccatcaccaaacagggcagcaccaacaaggtcataaggacggtcataactgggtggccggaagcctagcacagtggtctcagcagccgttactaggacacggctccctgggttgttctcagcgatgtccttggcagtgcggagggcagcagcaccaccggagcagccaaggaagagaagggaagtgcgcacggtgtttggactaaggccaaggcgagcagccaggtgaaggtcaccccccgggagacgaagttcactggatgagatgtagatgaggtgggtgatatcagctgcagggcggccccattcgtggagggcagcacgagctgcagcagcaccaaggtcgatcaccgcggcattgcagatgtcaaggcggggtgtcaatgttggagtaccctccatcttcagctctgggtgcttatccagaagctccttggacataacagtgtaccttgtcctcactgtggtggtcgtgcctggaaagtaaaatcatttggaaaatactcatctgcttactagaggcaaatcaaatcatcacatagatactcaaggtaaagataaagaccaatttagcatacaaaggcgctcgagcttggccctggtgtcgggatcgtcgcagctgctttcttggaggtagctcgccgcaaccttctcctggggcaggacttggtcagggaagcccttgcccaaggcgagcagcatcgccttgccccttgagccctggctcttgccgttggcgtccgtgctgccagtgtccgtcattgtcaccgcacgggcgctccttggtccacctttctgtttccagtgctgtactgttgtggttttcttggtgcggccaggccatggcaaacacatcttcggtgcttttaatgggtggcgcggtttgcggaagtgtttgtttggtttcggttctgcaggaggacggaggaggcatgggtgaaaacatggtaggtcgcggactggtcgtctcactctcgcttcttttctggggggagatggttcattctttccacccccttctttggtctgcccagtgtttttgcccatccagttggtgtgatctttcctacttgtccttatatggtcgtcccgcaggtacgagcgtgaagtgatgctagaaaatgtaacgctgttcagtttacatgttggttgatgctgaagccttgtggctaggtgctatgtcctatgatgttttgctgcagatgtttcttttagcgcaatgtggcaggtgttctgtcgttgcatttcatgtgctaggaattgggcatatgttcaaaggtctcagccgtcgaactgaactgac

SEQ ID NO: 6 - DNA, 3500 bp, Triticum aestivum
  source 1..3500: mol_type = other DNA
cccgggagtagcatgtttagtccgacagcccctgttcttcacccacaaccgggcctcgtcctggtcatgtcgagcaaaccggcgatgtgaggctacgcattgtctgtagaagactgctgtgtgcaacagatgctcacgacgagtatacagagtatcgccaaagataagaaacggaagcatgcagctgcagtcaaacaggtggcgatgttgggagtgaccgaacaattctacctcgtcttgggcagtttttgaagccaatgctagccgtgccgcttggtacaggccgtctagagagcgcacgcacggtgtgggcaaggatcgtcggagccagggagccgtgtcgcgcgggcttgagttgggcgccgagtgtgtgcatataacaatcctggcacggtggcacaagttgcgcgcagaccgcgtattaggcgtcccaggcacaagcagcaacagaagagaacctccaggtttcagctccagaacaaccttgtagtgatgttgaaaactgacaatttccaagaaacagtatacccaccgaagagaagtgaagagtcactgctcatactagtaacaaactgtaaccacatttcacgagcaagtgtgtctgcctgcccacagactccagcatcaaccacgtttcaggtaaacaaacaagcacgagacatttttacaacagggcacggggtcacatgaaaaaagatcacaccgaatagatgggcaaaaactatgggcgagccaaagaagggggtgtgaagaacgaaccttcgccgagccgcccttccacaaaaactggaagaaaaccagatgaccaatccacaacctaccatccttttcagtttacactcatgccgtcgttccctcaggcctaacaccaaacaacccgctccctgcaagcctgaacccaaacagccccatggcgagcatcccctattaaagcggagcacccatcatgcacacagcgaaatcacaggagcggagaacttgtaaacctgaagcggaaggaagcaaggagcgactgtgaggcagcgatgtctggcctggtggagtcgggagcattggcggaggcggcaacggcagaagccaaggcttggggggcaaggccaagctgctcgcgctcggcaagggactccctgaacaagttctaccccaggagaagctcgtggagacctacctccaggacaccagctgcgacgatcccgccaccagggccaagctcgaacgcctctgtgagttcccttccttcatgtcatttgtagttttacatataacagcttcccgggctgagagttttaagttggcaacaatagttgttctctgtcgacttcgaaaacaaaaatagttgattctccgtaaagagatcattgacactagtgagttgtagtaatactgccatttggttgcagcatcttagtttacagaatctaccatgctttagtataactgtcagttatcataggtgcaaagtgcgacaaatctccactcggtaaataaaatgttaacatggtcattattactaaatcccagttacagtttagcttaagaaaatcagcagtacctaacctattttatagagtatataaattgtgtaaaatggaattagtgtgatgagaaacacaacaaaattccacctagaaacaaaattccacaagaaaagactgtaggaaaactcgcctacagtcattgaccgctccaaaaaaaatccacctagaaacaacatgcatgcacaactagaattattactctccaattgtacttattttttgctctaggagaaacactaggacacccctcaaaaaagaacactagaacaccatttgtttttgtccttctgtttgtggtgagggctaagcaaataagagttaatagtacataatcacaaactataaactatacttatttgttatgtattccctccgtttggaaataagtgacgtggttttagttcaaatttgaactaaacccacaacacttatttccgaattgagggagtagtaatttgcatcagtccctccaatgtatgtataatggttcccctgtgatttgatttgacaggcaagaccacaacagtgaggacaaggtacactgtcatgtcgaaggagctcctggatgaacacccagaattgaagacagaaggcactccaacattgacgccacggcttgacatctgcaatgccgcggtgcttgaacttggtgctgctgcagctcgtgccgcccttggtgagtggggccgtccggcagctgatattacccacctggtctacatctcctccagcgagcttcgcctcccagggggtgatcttttcctggcaacccgtcttggcctctctccaaacacagtgcgcacatcccttctcttccttggctgctcaggtggtgctgccgccctccgcactgccaaggacattgcagagaacaacccagggagccgtgtccttgtaatagcggcggagacaactgtgttgggcttccggccgccaagccctgatcgtccttatgaccttgttggtgctgctctgtttggtgatggtgcatcagctgcaattattggagcaagccctatcaaagcagaagaggatcctttcttggagcttgagttctcaacccaggagttcctaccagggacagacaaggtaattgatggcaaaatctcagaggaagggattaacttcaaactggggcgtgatttgcctgaaaagattgaaagccgcatagaaggcttctgcaggatactcatggacaaggttgggataaaggagttcaatgatgtattttgggctgtgcatcccggtggaccagcaatattgaacaggctggaggtttgtcttcaacttgagccagataaactcaagatcagtagaaaggccctgatgaactatgggaatgtgagcagtaacacagtcttctatgtgctggagtatttgagggatgaattgaagaaaggggcaataagggaagagtggggattgatcttggcttttggcccaggcatcacatttgaaggactgctagttcggggcattaactgaaattgaaggagatccaagtaggttactaccagatatatggaaaggtcacaaacaagctttacatggcacggtagcatttcacaattataattatacagtgtaaggatagcaattcgcgactataattatatactgtaagaatatcataagcatagcgtcttgatgatttctgttcactacatactcagtactttgtaattttatgctttatgctatcaattatcaaacaatgtcagttactcgagtttgttcccaagactgtataagctgtgatttttttttaatctgtgctggttgtacaatgtacaagtgcatgaccaaactagtactggtacaactaccttcctctcagaaggaaaacgaagcagaacaactgaggcttgaaacataccattatcataaaactattctgaattacactaaacaaacggttacagtgatagatagtagatagtatcatgattcccttagcacttc

SEQ ID NO: 7 - DNA, 3999 bp, Triticum aestivum
  source 1..3999: mol_type = other DNA
tactcaccacaactgggcctcggtcctggtcgtgtcgagcaaaccggcgatgtgcaacagaaggtcacgagcacatagagaatatcgccgaagataacaaacggaagcatgcagatgcagtttaacaggcggccatgttggagtgacccaacaattctccctcatctttgcagtttttcaagccaatgctggctgtgccgcttggtccaggccgcacgcacggtgtgggcaaggattcatcggagccagggagccatgccgcgaggccgcgggcacgggcttgggttgggggcgtgccaaaagccacgccgagtgtgtgcatataacaatcctggcacggtggcagaagttgtggacagactgcatattaggcgtcccaggcacaagcagcaacagaagagaacctccaggtttcagctccaaaaccttgtagtgatgttgaaaactgacaatttctaagaaacattatacccacctaagagaagtggagagacacagctcagtgctcatactaacaaactgtaaccacatttcacgagcaagtgtgtctgcctgcccacagactccagcatcaaccacgtttcaggtaaacaaacaagcatgagacatttttacaacagggcatggggtcacatgaaaaaagatcacaccgaatagatgggcaaaaactatgggcgagccaaagaagggggtgagaggaacgaaccttcgccgagccgcccttccacaaaaactggaagaaaaccagatgaccagtccacaacctaccatccttttcggttttcactcacaccgtcgttccctcaggcctaacaccaaacaacccgttccctgcaagcctgaacccaaacagccccatggcgagcatcccctattaaagcagcacgcccatcacgcacacagcgaaatcacaggagcggagaacttgtaagcctgaaacggaagcaagcaaggagcgcctgtgaggcagcgatgtctggctggtggactcgggagcgtcggcgggggcagcaacggcagaagccatggctcggggggcaaggccaagctgctcgcgctcggcaagggtctccctgagcaagttctaccccaggagaagctcgtcgagacctacctccaggacaccagctgcgacgatcccgccaccagggccaagctcgagcgcctctgtgagttcccttccttcatgtcatctgcagttttacatacctgcttctcgggctgaggcatttaagttggcaacaatagttggctctccttaaagagatcactggcaacagtgagttgtaataaggttgccatctggttgtaacatcttagtgtacagaatctaccatgctttagtttaactatcagttatcataggtgcaaagtgcaattagtacaatgggaaagacaatcaatctccactcagtaaataaataaaatgttcacatggtcattagtactaaatcccggtcacagtttaggttaagaaaatcagcagtatctaaactattttacagtataaatcatgtgtaaaatggaattagtgtgatgagaaacacaacaaaatgccacctagaaacaaaattccacaagaaaagactaggagaactctcctacggtcattcaccgctcaaaaaaaaatccacctagaaacaacatgcatgcacaactagaattattactctccaattgtaattattttttgctctaggagaaacaccccctcaaataagaacactagaacaccatttattttcctccttctgtttgtggtgagggctaagcaaataagagttaatagtacataatcacaaactataaactatacttatttgttatttgaactaaaaccacgtcacttatttccgaatggagggagtagtaatttgcatcagtccctccaatgtatgtataatggttcccctgtgatttgatttgacaggcaagaccacaacagtgaggacaaggtacactgtcatgtcgaaggagctcctggatgaacacccagaattgaagacagaaggcactccaacattgacaccacggcttgacatctgcaatgccgcggtgcttgaacttggtgctgctgcagctcgtgccgcccttggtgagtggggccgtccagcagctgatattacccacctggtctacatctcctccagcgagcttcgcctcccggggggtgatcttttcctggcaacccgccttggcctctctccaaacacagtgcgcacatcccttctcttccttggctgctcaggtggtgctgccgccctccgcactgccaaggacattgcagagaacaacccagggagccgtgtccttgtaatagcggcggagacaactgtgttgggcttccggccgccaagccctgatcgtccttatgaccttgttggtgctgctctgtttggtgatggtgcatcagctgcaattattggagcaagccctatcaaagcagaagaggatcctttcttggagcttgagttctcaacccaggagttcctaccagggacagacaaggtaattgatggcaagatctcagaggaagggattaacttcaaactggggcgtgatttgcctgaaaagattgaaagccgcatagaaggcttctgcaggatactcatggacaaggttgggataaaggaattcaatgatgtattttgggctgtgcatcctggtggaccagcaatattgaacaggctggaggtttgtcttgaacttgagccaggtaaactcaagatcagtagaaaggccctgatgaactatgggaatgtgagcagcaacacagtcttctatgtgctggagtatttgagggatgaattgaagaaaggggcaataagggaagagtggggattgatcttggcttttggcccaggcatcacatttgaaggaatgctagttcggggcactaactgaaatcgaaggagatccaagtaggttactaccagatacatggaaagatcacaaacaagctttacatggcacagtagcatttcacaattataattatacagtgtaaggatagcacttcgcgactataattatatactctaagaatatcataagcatagcgtcttgatgatttctgttcactacatactcagtactttgtaattttatgctttatgctatcaattatgaaacaatgtcggttactcaagtttgttcccaagactgtataagctgtgattttttttttaatctgtgctggttatacaatgtacaagtgcatgagcaaactaatactggtacaactaccttcctctcagaaggaaaaagaagcagaacaactgaggcttgaaacataccattatcataaaactattctgaattatactaagcaaacggttacagtgataggatagtagatagtatcatgatccccttagcacttcaacaatgtaatatctttttaagtattaccaaaaggcaacaaaatagaagaagctcttacagaaaagggattacgaaatggcactgaccattctgccttatagagacattacactctgatctaactattggaccttgaaagataggcccatgaagacaatactagaacccatgtgaatgcccttcttgttcatcaacgttattcaaaggccaaaacacttgaataaatcagaactgacttgcatgcctccaacaaatcatggatctgaacttggtgctccctcttcaagagataactcaccagcaatcttctcccaagcaactttagcatcccctttcacaagaaagttcatcttggcaacaacacccttcgcctctgcagacctccctcccttgaccaacccctcgactaaacccttgacagcctcaaatgatggggctgccttcattgcaaagcagctgttacaaagccacagtgcatcatcaagcctttccttctctaccaaactcttaaacagcaggacatatgtctgggagtgcaccgacactcccaagtcactcctcccttcccgcctcttcatcaccttcatcctcttgaatactcgcatggcggccccg

SEQ ID NO: 8 - DNA, 3000 bp, Triticum aestivum
  source 1..3000: mol_type = other DNA
ccgcgccgagtgtgtgcatataacaaccctggcacggtggcagaagttgtggcgtcccaggcacaagcagcaacagaacagaacctccaggtttcagctccagaaccttataaccatatcacaaactgacaatttctgagaatcagtatacccaactaagacaaacgaagagacactgctcatatactactaacaaacagcaagagtgtctgcctgcccagagactccagcatcaaccacgtttcaggtaaacaagcatgagacagttttacaacagggcatgggggctcacacgaagatcacaccgaatagatgggcaaaaaccatgggcgaaccaaagaaggggggtgggagaacaaacctgcgcgagccgcccttccataaaaactggaagaaaaccagatgaccagtccacaacctaccacccttttcagttcacactcatgccgtcgttccctcaggcctaacaccaaacaacccgctcccagcaagcctgaacccaaacagccccatggcgagcatcccctattaaagcgaagcacccatcacgcacacagtgaaatcacaggagcggagaacttgtaaacctgaagcggaagcaagcaaggagcgcctgtgaggcagcgatgtctggcctggtggactcgggagcatcggcgggggcggcaacggcataagccacggctcggggggcaaggccaagctgctcgcactcggcaagggtctccctgaacaagttctaccccaggagaagctcgtcgagacctacctccaggacaccagctgcgacgatcccgccaccagggccaagctcgaacgcctctgtgagcttccttccttcatgtcatttgtagttttacatataactgcttctcgggctgagagttttaagttggcagcaatagttgttctccgtcgacttcgaaaacaaaaatagttgattctccgtaaagagatcattgccatttggttgtagcatcttagtttacagaatctaccatgctttagtataactgtcagttatcataggtgcaaagtgcaacgaatctccactcggtaaataaaatgttcacatggtcattattactaaatcccagttacagtttagctcaagaaaatcagcagtacctaacctattttagagtatgtaaatcatgtgtaaaatggaattagtgtgatgagaaacacaacaaaattctacctagaaacaaaatttcacaagaaaagagactgtaggaaaactctcctacagtcattgaccgctaaaaaaaaaaatccacctagaaacaacatgcgtgcgcaactagaattattactctccaattgtacttattttttgctctaggacaaacactagaccccctcaaaaaagaacactagaacaccatttgtttttgtccttctatttgtggtgagggctaagcaaacaagagttaatagtacataatcacaaactataaactatacttatttgttatgtattccctccgttcggaaataagtgacatggttttagttcaaatttgaactaaactcacaacacttatttccgaattgaggaagtagtaatttgcatcagtccctccaatgtatgtataattgttcccctgtgatttgatttgacaggcaagaccacaacagtgaggacaaggtacactgtcatgtcgaaggagctcctggatgaatacccagaattgaagacagaaggctctccaacattgacgccacggcttgacatctgcaatgccgcggtgcttgaacttggtgctgctgcagctcgtgccgcccttggtgagtggggccgtccagcagctgatattacccacctggtctacatctcctccagcgagcttcgcctcccagggggtgatcttttcctggcaacccgtcttggcctctctccaaacacagtgcgcacatcccttctcttccttggctgctcaggtggtgctgccgccctccgcactgccaaggacattgcagagaacaacccagggagccgtgtccttgtaatagcggcggagacaactgtgttgggcttccggccgccaagccctgatcgtccttatgacctcgttggtgctgctctgtttggtgatggtgcatcagctgcaattattggagcaagccctatcaaagcagaagaggatcctttcttggagcttgagttctcaacccaggagttcctaccagggacagacaaggtaattgatggcaagatctcagaggaagggattaacttcaaactggggcgtgatttgcctgaaaagattgaaagccgcatagaaggcttctgcaggatactcatggacaaggttgggataaaggagttcaatgacgtattttgggctgtgcatcccggcggaccagcaatattgaacaggctggaggtttgtcttgaacttgagccagataaactcaagatcagtagaaaggccctgatgaactatgggaatgtgagcagcaacacagtcttctatgtgctggagtatttgagggatgaattgaagaaaggggcaataagggaagagtggggattgatcttggcttttggcccaggcatcacatttgaaggaatgctagttcggggcattaactgaaattgaaggagatccaagtaggttactaccagatacatggaaagatcaaaaacaagctttatatggcacagtagcatttcacaattataattatacagtgtaaggatagcaattcgcggctataattatatactgtaagaatatcataagcatagcgtcttgatgatttctgttcactacatactcagcactttgtaattttatgctttatgctatcaattatcaaacaatgtcagttactcgagtttgttcccaagactgtataagctgttatttttttaatctgtgctggttgtacaatgtacaagtgtatgagcaaactaatactggtactaccttcctctcagaaggaaaaagaagcagaacaactgaggcttgaaacataccattatataaaact

SEQ ID NO: 9 - DNA, 20 bp, synthetic construct
  source 1..20: mol_type = other DNA
gcagcgatgtctggccttgg

SEQ ID NO: 10 - DNA, 20 bp, synthetic construct
  source 1..20: mol_type = other DNA
gatcgtcgcagctggtgtcc

SEQ ID NO: 11 - DNA, 20 bp, unidentified
  source 1..20: mol_type = unassigned DNA
gatcgtcgcagctggtgtcc

SEQ ID NO: 12 - DNA, 20258 bp, synthetic construct
  source 1..20258: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttcggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagcctagtaagccgccatggccccaaagaagaagaggaaggtcggcatccacggcgtcccagctgcgatggacaagaagtactccatcggcctcgacatcggcaccaacagcgtgggctgggccgtcatcaccgacgagtacaaggtgccatccaagaagttcaaggtcctgggcaacaccgaccgccacagcatcaagaagaacctcatcggcgctctcctgttcgactccggcgagacggctgaggctaccaggctcaagcgcaccgccaggaggaggtacaccaggaggaagaacaggatctgctacctccaagagatcttctccaacgagatggccaaggtggacgactccttcttccaccgcctggaggagagcttcctcgtcgaggaggacaagaagcacgagaggcacccaatcttcggcaacatcgtggacgaggtcgcctaccacgagaagtacccaaccatctaccacctgaggaagaagctcgtggactccaccgacaaggccgacctccgcctgatctacctcgccctggcccacatgatcaagttcaggggccacttcctgatcgagggcgacctcaacccagacaacagcgacgtggacaagctgttcatccaactcgtccagacctacaaccagctcttcgaggagaacccgatcaacgcttccggcgtggacgctaaggctatcctgagcgctaggctctccaagagcaggaggctcgagaacctgatcgcccagctcccaggcgagaagaagaacggcctgttcggcaacctcatcgctctctccctgggcctcaccccaaacttcaagagcaacttcgacctcgccgaggacgccaagctgcaactctccaaggacacctacgacgacgacctggacaacctcctggcccagatcggcgaccaatacgccgacctgttcctcgccgccaagaacctgtccgacgccatcctcctgagcgacatcctccgcgtgaacaccgagatcaccaaggccccactctccgccagcatgatcaagcgctacgacgagcaccaccaggacctgaccctcctgaaggccctggtcaggcaacagctcccagagaagtacaaggagatcttcttcgaccagagcaagaacggctacgctggctacatcgacggcggcgcctcccaagaggagttctacaagttcatcaagccaatcctggagaagatggacggcaccgaggagctcctggtgaagctcaacagggaggacctcctgaggaagcagcgcaccttcgacaacggcagcatcccacaccaaatccacctcggcgagctgcacgctatcctgaggaggcaagaggacttctacccattcctcaaggacaacagggagaagatcgagaagatcctgaccttccgcatcccctactacgtcggcccactcgctaggggcaactccaggttcgcttggatgacccgcaagagcgaggagacgatcaccccgtggaacttcgaggaggtcgtcgacaagggcgcttccgctcagagcttcatcgagaggatgaccaacttcgacaagaacctgccaaacgagaaggtgctcccaaagcactccctcctgtacgagtacttcaccgtctacaacgagctcaccaaggtgaagtatgtgaccgagggcatgcgcaagccagccttcctgagcggcgagcagaagaaggccatcgtggacctcctgttcaagaccaacaggaaggtgaccgtcaagcaactcaaggaggactacttcaagaagatcgagtgcttcgactccgtggagatcagcggcgtcgaggaccgcttcaacgcctccctcggcacctaccacgacctcctgaagatcatcaaggacaaggacttcctggacaacgaggagaacgaggacatcctcgaggacatcgtgctgaccctcaccctgttcgaggacagggagatgatcgaggagcgcctgaagacctacgcccacctcttcgacgacaaggtcatgaagcaactcaagaggaggaggtacaccggctggggcaggctgagccgcaagctcatcaacggcatccgcgacaagcagtccggcaagaccatcctcgacttcctgaagagcgacggcttcgccaacaggaacttcatgcaactgatccacgacgactccctcaccttcaaggaggacatccaaaaggctcaggtgtccggccagggcgacagcctgcacgagcacatcgctaacctcgctggcagcccagccatcaagaagggcatcctgcagaccgtgaaggtcgtcgacgagctcgtgaaggtcatgggcaggcacaagccagagaacatcgtcatcgagatggcccgcgagaaccagaccacccagaagggccaaaagaactccagggagcgcatgaagcgcatcgaggagggcatcaaggagctgggcagccaaatcctcaaggagcacccagtggagaacacccaactgcagaacgagaagctctacctgtactacctccagaacggcagggacatgtatgtggaccaagagctggacatcaaccgcctctccgactacgacgtggaccacatcgtcccacagtccttcctgaaggacgacagcatcgacaacaaggtgctcaccaggagcgacaagaaccgcggcaagtccgacaacgtcccaagcgaggaggtggtcaagaagatgaaaaactactggaggcagctcctgaacgccaagctgatcacccaaaggaagttcgacaacctcaccaaggctgagaggggcggcctctccgagctggacaaggccggcttcattaaaaggcagctggtggagacgcgccaaatcaccaagcacgtcgcccaaatcctcgacagccgcatgaacaccaagtacgacgagaacgacaagctgatcagggaggtgaaggtcatcaccctgaagtccaagctcgtgagcgacttcaggaaggacttccagttctacaaggtccgcgagatcaataattaccaccacgcccacgacgcttacctcaacgctgtggtcggcaccgccctgattaaaaagtacccaaagctcgagtccgagttcgtgtacggcgactacaaggtgtacgacgtccgcaagatgatcgccaagtccgagcaagagatcggcaaggccaccgccaagtacttcttctacagcaacatcatgaacttcttcaagaccgagatcaccctggccaacggcgagatcaggaagcgcccactcatcgagacgaacggcgagacgggcgagatcgtgtgggacaagggcagggacttcgccaccgtgcgcaaggtcctctccatgccacaggtgaacatcgtcaagaagaccgaggtccaaaccggcggcttctccaaggagagcatcctgccaaagaggaacagcgacaagctcatcgcccgcaagaaggactgggatccaaagaagtacggcggcttcgactccccaaccgtggcctacagcgtcctcgtggtcgccaaggtggagaagggcaagtccaagaagctgaagagcgtgaaggagctcgtcggcatcaccatcatggagaggtccagcttcgagaagaacccagtggacttcctcgaggccaagggctacaaggaggtcaagaaggacctgatcattaaactcccaaagtacagcctcttcgagctggagaacggcaggaagcgcatgctggcttccgctggcgagctccaaaagggcaacgagctcgccctgccatccaagtatgtgaacttcctctacctggcctcccactacgagaagctcaagggcagcccagaggacaacgagcaaaagcagctgttcgtcgagcagcacaagcactacctcgacgagatcatcgagcaaatctccgagttcagcaagcgcgtgatcctcgccgacgccaacctggacaaggtcctctccgcctacaacaagcacagggacaagccaatccgcgagcaggccgagaacatcatccacctcttcaccctgaccaacctcggcgctccagctgccttcaagtacttcgacaccaccatcgacaggaagcgctacacctccaccaaggaggtgctggacgccaccctcatccaccagtccatcaccggcctctacgagacgaggatcgacctgagccaactcggcggcgactccagcccaccaaagaagaagaggaaggtcagctggaaggacgcttccggctggagccgcatgtgaggtacctcacatcgatcgacgaccaaggatatgattattatctatctagcttgtggtggtggttgaacaataataagcgaggccgagctggctgccatacataggtattgtgtggtgtgtgtgagagagagagaaacagagttcttcagtttgctatctctctctgcatgtttggcgtcagtctttgtgctcatgtacgtacgtgtgtctacctgcatgttggttgatccgattgcatctgctgtaaccatatattaattggtccacgatgatatgatttgatactatatatatactaaaaccggacttcttattataatacttgtagtatataagtttcttacgcccgcaattgatcgattcagaaggagttctagctagctaaaacatgcagattcagaatatcagatttttaggactactggagatccagaaccttcgtgtccttgtacccgtgattttggatcccctttctccccactacaatcgttggcgcaatcttgttctgctcgcctagaatggtacgccctcaccgaccatgtcctcgccgatgcctaccccgccacgccatctggatggtgcatgaatgtcgtcgttttgtcctggatgctaggcacactctcccccgagttgatggagcctcctcgcacctctggtggcactgcgccgcgcctggcttgccatcgaggagcaattcctcggcaatcgcgaagctcgcacacttcgtctcgacgccgagttccatgtcttcatgtaggcgatctcttcgtcagcgactattgtctattgacgcaagatgaaggggatggacgaggcccttggtgatcttggtgaggtcatccatgaccgtacccttgtcctaaacgtgttgtgtggtctgaatgagaggtttgcccacataaaggtccacttcaagcactcgaatccgttcccctccttcaccgacgtttgtaatgatctcatccttgaggagatcgactccagcgcgcctcctccgcctccgaccacctaattagctaagggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaggatttggtgcctgatgagtccgtgaggacgaaacgagtaagctcgtcgcaccacagcatagcaagttaaaataaggctagtccgttatcaacttgaaaaagtggcaccgagtcggtgcttggccggcatggtcccagcctcctcgctggcgccggctgggcaacatgcttcggcatggcgaatgggactaattagtggtgcctgatgagtccgtgaggacgaaacgagtaagctcgtcgcaccacagcatagcaagttaaaataaggctagtccgttatcaacttgaaaaagtggcaccgagtcggtgcttggccggcatggtcccagcctcctcgctggcgccggctgggcaacatgcttcggcatggcgaatgggacagatctaattaactagcgctgcctgatgagtccgtgaggacgaaacgagtaagctcgtcgcagcgatgtctggccttgggttttagagctatgctgtggtgggccggcatggtcccagcctcctcgctggcgccggctgggcaacatgcttcggcatggcgaatgggactagttaaacgatcctgatgagtccgtgaggacgaaacgagtaagctcgtcgatcgtcgcagctggtgtccgttttagagctatgctgtggtgggccggcatggtcccagcctcctcgctggcgccggctgggcaacatgcttcggcatggcgaatgggacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcaatagactacggaccttcgaactaggctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgactaaatagatgcagaagctgatcaacagcgtgcagaactacgcctggggcagcaagaccgccctgaccgagctgtacggcatggagaaccccagcagccagcccatggccgagctgtggatgggcgcccaccccaagagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatccccatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagaccccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagaccactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgatagctacgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcttaagcctgcaggcggaccgcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 13 - DNA, 6001 bp, Triticum aestivum
  source 1..6001: mol_type = genomic DNA
cctattacaaagatggataaaacctatattgctttctgcaaagaaaaccaaccaacacttaccctaaaagcatggttgggtttgttatacttattggtgaagcttgcgagtacattcaatgtattgaaccttgtggttgcaacgtagcatgttgcatgacttatgcataatgagcaaggattatgaactaggcttacgattctatactccacttgtacctgtgagtggatggacataacaagataatattgtattttgttgtattttattgtaagctagccttgagctaagtagatacgtaatgtaataaagactagatcagcaatgtaatactcatgacatgtggttatcttaatatttgatcgtgtaatgtgtgtgctagcaattgatctagcttgtgactagcacaaaagcgcaaatagtctgttttcttatcagggaccgggtcgctacaagcagagtaccactgtgtagcaaatacaatggctgaatgtttttgactccggcagatactcgtcaaactccattgctccatcaacaaggtaattgttgtcttctgcgacaatgtatctaccatatacctctacatcaaccccgttcatcatcaacgtacacattgagttagacattcacttcgtacgcgaaaaggtctctctcttggacagttccgtgtcgctcatgtgcctactccgggacagcttacagatatcatgacgaaagggctaccgtcggcaagctttgaaggatttcgttccagccttggcatcatctctgatgacctgcgactgcttgggtggtgtgagagaagataacgatggggctccatattgcatgcgtttctccgtaccaatcctatatcttcattgttacgtacaatcttgcatagattctttagtttctttattaaaaaccgtgattttttttaggtgtcaaaaaccgtgattagatttagagattaggtgggttgtacaatgggctgggcaattcccaaacctacacggacgagcacgtggaagtgggaaggcccaaggccaagcaacccaactaactcccacttggggcggattggttggtagcaatggtaggtagacgtgtcgctcgctcccatctcctatccgcgcattcgtggccatccatcctcccgtccaatggacctaaccccgtcctcccaacggccagctttcccttacttacccacccttcccgcctatatatcccgtcgcccgcactctcatggcaccacaacacaccacgccagtacaagcggcgcctgtcatttgtctaccactctgctagctgcttccctctgcgtccggcgaacttagacgtcacacagccggccactggtgcgacaactcctagctcgtcggccgctggtacgtgcctacgtagatccatcgatggcggccaccatgaccgtggaggaggtgaggaaggcgcagcgggcggaggggccggccaccgtgctcgccatcggcacggcgacgcccgcgaactgcgtgtaccaggccgactacccggactactacttcaagatcaccaagagcgaccacatggccgacctcaaggagaagttcaagaggatgtgtaagtctatgttcaccgtctctgtgttcgggcgtgcatgcacggcttgtttcccataacggcgctactatacagtaatatacatgtgtctgcatggatgtcaatattttactccttatattaattcccgtaaacttttgttgatagttagaaaataagtactgggaggcacatcgatcactagcaataagccaagtactagccatggatcggagtagcatatatacgtgaaacgacgaacacaataagcatggccatgaaaactgggtaacggccgcacacgccgaacctgttgtccatttaactgtttattctatttttagctgcaagtgtttatattaattcccgtatacttttttttgtgaaatatgtatactcctgcacgtaattcttttttgataaccctgcacgtgggtagcacactcgagtgtctcgcctcctactccctccattcctaaatataaatatttgtagagatttcattatgaaccacataaggatgtatatggatgcattttagagtgtatattcactcatttcgctccgtatgtagtccatagaagaatctccacaaagccttatatttacgaatggaaggagtacatatcagtagcatgcatgcatgtttaaaggtcaacactcaacagtgttacagtggatcggttaacctacgaacgggttgattacagaacgtgtttatatgtggactaatttccttagatgcaccaaaattaattaccggttggtcgaacaatatgttcccatatctcatgtatttaattttttcagttgaagcatttaaatagaaaacaaaactgattttcagaagaaaacaacgacaacggtttttttttacagggtaaagggagttttattccatatagtgacaagattgcactcctaattaatacaagatcgtgagttgacaatgatttgcaaaatatctaatagtagtattgcgttgatttcctgaaaagaatatcagttttacaaaaaatagacatgtttgcatgaaccgccaaaaaactaataatttccaaagacaagcaattaatccagaatgtcactaccggacgcgattatgacttgacactgtacctagaacttttcttttgagtgcactctgtacctagaactagtgggccactcgacaaagaaagtaccagcaagtactggaaacatgagcgaatccttgagttgacttgagagctaatctctacattagtttcttcaaatagaaattaacctcctacattaaagagagagagaaacccaaatgtagacatcgcttgcacacctaattaacccatgaatacttgtctcctgtctggtttgccttttcttggtctggacacgcgagcgagctaactctgcctgcctgcgggccttgattcgatttgcaggtgacaagtcgcagatcaggaagagatatatgcacctaacggaggagatccttcaggagaaccccaacatgtgcgcctacatggcgccctccctggacgcgcgccaggacatcgtcgtcgtcgaggtccccaagctcggcaaggcggccgcgcagaaggccatcaaggagtggggccagccgcgctccaagatcacccacctcgtcttctgcaccacctctggcgtcgacatgcccggcgccgactaccagctcaccaagatgctcggcctccgcccctccgtcaagcgcctcatgatgtaccagcagggctgcttcgccggcggcaccgtgctccgcctcgccaaggacctcgccgagaacaaccgcggcgcgcgcgtgctcgtcgtctgctccgagatcaccgccgtcaccttccgcggcccgcacgagtcccacctcgactccctcgtcggccaggcgctcttcggcgatggtgcggccgcggtgatcatcggcgccgaccccgacgtgtccgtggagcgtcccctgttccagctcgtgtccgcgagccagaccatcctgccggactcggagggcgccatcgacggccacctccgggaggtcggcctcaccttccacctgctcaaggacgtgcccgggctcatctccaagaacatcgagcgcgccctagaggaggccttcaagccactcggcatcgacgactggaactccgtcttctggatagcgcacccaggcggcccggcgatccttgacatggtggaggccaaggtaaacctccacaaggagcggatgcgtgcgaccaggcatgtcctctccgaatacggcaacatgtccagtgcctgcgtcctcttcatcatggatgagatgcgcaagcgctccgccgaggatggccacgccacaaccggcgagggaatggactggggcgtcctcttcggcttcggccccggcctcacggtggagaccgtcgtcctccacagcgtccccatcaccgccggggcaaccgcatgatcatcgctccttatccatttatcttcctcatgcatggaaaaaacctacgtactacgtacagcagcttctacactgccgatcgagtttgtaccatatatattctaccgtacgtgcgtggtttgtatgtgttggtagaagatgcattctcctgttagtctgtgtcgcgcacgccgttgcatactttctagtagtaacacaaccgagcttttacaggtttattataagcaaagtgtggtcataatgtggagtgtgaggtgtattacctgtgtacgaaaagattggctgttcaatttgaaccttatgtggttggaaaacaaatggtgattctttcaaagctttattgaaatacatattctcctctctcctactttttgtttatgaatataatgctttatttaattcataaatgcagtgtcaaaagcgtttttatattatgggacggagggagtacaaaccataacgaacacactcgtccggcctctaaaagtacccagactcactataggaatcatatagtccgacacatattactcggcaagcacatatagcatgacaacctcataggtccatatatagaagcaacaagtcacatatataagtatacaacactctccaacaagcatacatatagcatgacaacctcatataggtccatagcataataagaatacaagcaacatagatactccaagaacaagtttaaaaccaacaagcagcatagacactccaagaaatgcaatgagcttcaccgtcgacagcatccgcaatgaaagctacaagttcagaagcacatgcatgagtttgcaagttcaaatgtcaaacaagctactaggggtaagtttcaagctctttatcaaaagatgtgcacatctaagttagaatgtgttgcttatggcatttgtgagaatatattagtggtgtggaagttgtgtctatggacacaaaatcatataatttatgagaaagcgatgatgacatcaatggtggttgcttatctcatcgaaagaggtgacttttcattgcaatcaaatgcaagcaatcatatcaaaaactagcctggcatggagtcattattgcctggagatcaaaggggcatgagttgtaggcgtcactggcttccttgcgcagggccatatgcacctccttgcatattatattgttcgatccaacactcggattgagatgtaaacatgagacggtaagatgtattaggtaaggatgaaaatggtgattgaaaagtattagaagagaactcacaaagttgaagttaggaagaggaggaaggatcttgcagatctccgccttgcatagcccctatactcaacatacctttgttgcgcatgtatggttacatgttctaagctgattgatggatggaatcatagtatactatggtaaagaacttgtcatgaaaaggagtgtaaaccttccattgtaggctacatgttgtagtgccaatatctcctacatctgctggatttgaggtgaccttcctccgtctttctttgggcctcttccacctatctcttgggctcctccaatagtcccgcttcttcatccacgcgggcctacattatgtcctcgcagggtcatcaccgttgcaatgcaatcatgtaattatgtgtaagatcaatgaagaagaacataactgcattgagttaagcaactgccgccagctccgacaaaaaaattgacttttgatcatcgtcagatggtccataaaccgtggtcatccaaaatgattttctactactaatgatctctacttgaaccgtgatagataaactagctatggcgcgagaaactatgttcaccatccttcaacaaaacagagagaccatctgccacaaataaaaacaggaacggtgaaagtggatcactttgccgaagctcgcgcgatggtgcaaacgaatccaacagggttccattaaatttgaccgtgtatagtgaatcatatggtgttactccctctcaatatttgttgtgatagattgagtcttg

SEQ ID NO: 14 - DNA, 5001 bp, Triticum aestivum
  source 1..5001: mol_type = genomic DNA
catgtggttaccttaatatttgatcatgtaatgtgtgtgctggcaattgatctagctcgactagcacagaagcgcgaagagtatgttttcttaccaggggccgggttgctacaagcagagtaccactctatggctaatacagtggttgaatattcttgactccggcaattacttgtcagactccattgctccatcaacaaggtaattgttgtcttctgcgacaatgtatctaccgtgtacctctccgttaaccctgttcatcatcgacgtacacattgagctagacattcatttcgtacgcgaaaatgtctctctcttggacagtttcgttcacgtgcctactccgggacagcttacagatatcatgacgaaagggctagcgtcggcaagcttagaaggattttgttccaggcttggcatcatcataactccgactgcttgggtggtgtgagagaagatagcgatggggctccatattgcatgtgtttctccgtaccaatcctatatcttcattgttacaatcttgcatagattctgtagttttagtttctttaaaaaccgtgattagatgtagagattaggtggattgtacaatgggctgggcaattcccaaacctacacagacgagcacgtgcaactgggaaggcccaaggccaagcaacccaactaacccccacttggggcggattggttggtagcaatggtaggtagacgtgtcgctcggccctcatctccctatccccgcattcgtggccatccatcctcccgtccgatggacctaacccccttctcccaacggccagctttcgcttacttacccacccttcccgcctatatatcccgtcgcccatactctcatggcaccacaccacaccacgccaccagtacaagcggcgcctgtcatttgtctaccacctgctagctagctgcttccctctgcgccccgcaaacttagacttcacacagccggccactggtgcgacaactactagctcgtcggccgctggtacgtacgtagatcccatcgatggcggccaccatgaccgtggaggaggtgaggaaggcgcagcgggcggaggggccggccaccgtgctcgccatcggcacggcgacgccggcgaactgcgtgtaccaggccgactaccccgactactacttcaagatcaccaagagcgaccacatggccgacctcaaggagaagttcaagaggatgtgtaagtctatgttcaccatctgtgcgttcgggcgtgcatgcacggcttgtttcccctaacggcgctactatatactactccctctgtaaactaatataagagcatttagatcactactttagtgttctaaatgctcttatattaatttacagagggagtactatacatgtgtctgcatggatgtcaatattttactccttatattaatccccgtaaacttttgttgatagttataaaataagtactaggaggcaaacgatcactagcaataagccaagatgtgtactagacatggatcggagtagcatatacatatatacgtgaaactgacacaataagcatggccatggaaactgggtaacggccgcacacgccgaacctgttgtccatttaactgttgttctgttttagctgcaaaggtaggtaaatatgtatactcctgcacgtaattgttttttgataaccctgcacgtgggtagtacactcgagtgtctcgcctacatatcagtagcatgcatgtttaaaggtcaacactcaacagtgttacactggatcggttcgaccaagaattaattcctaaacctacggacagttgattacagaacgtgtttatatgtggactaatttcctcgttaatggcacatctaaatcatccttagatcccgtaaaaaaaaccatccttagatgtacctttaattaccggttggtcgaacaatatgttcccatattttttccccgcaaaaaaaaatatgtttccatatttcgtattttttttcaattgaagcatttaaatctttttatagataacaaaactaattttgagaagaaaaaaacaagaatggtttttttggcagggtaaagggagttttattattcgatagtgacaggattacattctgctaatacaagatcgtgaattgacaatgatttgcaaaatatctaatactagtattgcgttgatttcttgaaaaaatatcagttttacaaagaatagacatgtttgcatgaaccgccaataatttactacgtagtaaacaatttctaaagacaagcaattaatccagaatgtcgctaccggacgcgactatgacttgacactgtacctagaacttttcttttgagtgcacactgtacctagaactagtgggccactcgacaaagaaggtactagcaagtatagaaacatgagcgaatccatgagttgacttcacagctaatctcctacatgaatttcttcaaatactaattaaacctcctacattaaagggagaaaacccaaatatagacatcggttgcatacctaattaacccatgaatacttgtctcccgtctactttgccttttctcggttggacacgcgagctaactctgcctgccggccttgatttgcaggtgacaagtcgcagatcaggaagagatacatgcacctaacggaggagatccttcaggagaaccccaacatgtgcgcctacatggcgccctccctggacgcgcgccaggacatcgtcgtcgtggaggtccccaagctcggcaaggcggccgcgcagaaggccatcaaggagtggggccagccgcggtccaagatcacccacctcgtcttctgcaccacctccggcgtcgacatgcccggcgccgactaccagctcaccaagatgctcggcttgcgcccctccgtcaagcgcctcatgatgtaccagcagggctgcttcgccggcggcaccgtgctccgcctcgccaaggacctcgccgagaacaaccgcggcgcgcgcgtgctcgtcgtctgctccgagatcaccgcggtcaccttccgcggcccgcacgagtcccacctcgactcgctggtcggccaggcgctctttggcgatggcgcggccgcggtgatcgtcggcgccgaccccgacgtgtccgtggagcgtcccctgttccagctcgtgtccgcgagccagacgatcctgccggactccgagggcgccatcgacggccacctccgggaggtcgggctcaccttccacctgctcaaggacgtgcccgggctcatctccaagaacatcgagcgtgcgctagaggaggccttcaagccactgggcatcgacgactggaactccgtcttctggatagcgcacccaggcggcccggcgatccttgacatggtcgaggccaaggtaaacctcaacaaggagcggatgcgcgccaccaggcatgtcctctccgagtacggcaacatgtccagcgcctgtgtcctcttcatcatggatgagatgcgcaagcgctctgccgaggatggccacgccacgaccggcgagggaatggactggggcgtcctcttcggcttcggccccggcctcacggtggagaccgtcgtcctccacagcgtccccatcaccgccggggcaaccgcatgatcatcgctccttctccatttatcttccccatgcatggaaaacaacctactacatacagcttctactgccgagtttgtaccatatatattctaccgtgcgtggtttgtactatgtgttggtagatgcattcgatctcctgttagtctgtgtcgcgcacgccgttgcatacttactagtagtaacacaaccgagcttttacaggtttattataagcaaagtgtggtcataatgtggagtgtgaggtgtatttacctgtgtacgaaaagattggctgttcaatttgaaccttatgtggttggaaagcaaatggtgattctttcagagctttattgaaatacatattctcctatctcctaccttttgtttatgatgaatataatgcttcatttaattcataatgtagcgtcaaggaggtacaaaccataatgaacatactcctcgcctctaaaagaacccagcctcatgcatcaaattatctaccacacacacacaaaattgaatgtttctagtatttattttgattgttctcgtcagggtgcagctgagcctaggctcggaagtggattaccgatatataatcatatagtccaacacatattactcagcaagcacatatagcatgacaacctcataggtccatagaagcaacaagtcacatatatacaacactctccaacaagcatgcatatagcatgacaacctcatataggtccatagcataataagaatacaagcaacatagacactccaagaacaagtttaaaaccaacaagtagcatagacacgtcaagaaatgcaatgagcttcatcgtcgacagcgtccgcgatgaaacctacaagttcagaagcacatgcataagtttgcaagttcaaatgtcaaacgagctactaggggtaagtttcaagctctttatcaaaagatgtgcacacttaagttagaatgtgttgcttatggcatttgtgagaatatattagtggtgtggaagttgtgtctatggacacaaaatcatataatttatgataaagcggtgatgacatcaatggtggttgcttatctcatcgaaagaggtgacttttcattgcaatcaaatgcaagcaaacatatcaagaactagcctggcctggagtcattgttgcttggagatcgaaggggcatgagttgtaggtgtcactagcttccttgtgaagggctagatgcacctccttgaatattgttcgatccagcactcggattgagatgtaaacatgagacggtaagatgtatcaggtaacgatgaaaatggtgattgaaaagtatcggaagagaactcacaaagtggaagttaggaagaggaggaatgatcttgtagatctccgccttgcatagcccctatactcaacatacc

SEQ ID NO: 15 - DNA, 4001 bp, Triticum aestivum
  source 1..4001: mol_type = genomic DNA
taacgatggggctccatattgcatgcgtttctccgtaccaatcctatatcttcattgttacaatcttgcatatattctgtagttttagtttgtttaaaaaccttgattagatgtagagattaggtgggttgtacaatgggctgagcaattcccaaacctacacagacgagcacgtgcaagtgggaaggcccaaggccaagcaacccaactaactcccacttggggcggattggttggtagcaatggtaggtagacgtgtcgctcggccctcatctcctgcccgcgcattcgtggccatccatcctcccgtccaatggacctaacccccttctcccaacggccagctttcccttacttacccacccttcccgcctatatatcccgtcgcccatactcatggcaccacaccacaccagtacaagcagcgcctgtcatttgtctaccacctgctagctgcttccctctgcgtcccgcaaacttagacgtcacgcagccggccactggtgcgacaactcctagctcgtcggccgctggtacgtacgtagatccatcgatggcggccaccatgaccgtggaggaggtgaggaaggcgcagcgggcggaggggccggccaccgtgctcgccatcggtacggcgacgcccgcgaactgcgtgtaccaggccgactacccggactactacttcaagatcaccaagagcgaccacatggccgacctcaaggagaagttcaagaggatgtgtaagtctatattcaccatctctgtgttcgggcgtgcatgcacggctaacggcgctactatatagtacgtaatatacatgtgtctgcatggatgtcaatattttactccttatagtaatccccgtaaacttttgttgatagttagaaaataagtactaggaggcaaaccgatcagtagcaataagccaggatatttttacgagtagccatggatcggagtagcatatagacgtgaaacgacgaacacaataagcatggccatgaaactgggtaacggccgcacacgctgaacctgttgtccatgtaactgttgttctgttttagctacaaaggtaggtaaatatgtgcacgtaattgtcttttgataaccctgcacgtgggtagcacactggagtgtctcgcctacatatcagtagcatgcatgtttaaaggtcaacactcaacagtgttacagtggatcggttcggccaaaaattaattcctaaatctacggacaggttgattacataacgtgcttatatgtggactaatttcctcactgatggctcatctaaatcatccttagatgtaccattaattaccggttggtcgaacaatatgttcccatacttttttccccgcaaaaaaaatatgttctcatatttcgtatttttttttcagttgaagcatttaaatctttttatagatagcaaaactaattttgagaagaaaaaaacgagaatggtttttttggcatgaacggccaaaaaactaataatttctaaagacaagcaattaatccagaatgtcgctaccggacgcgactatgacttgacactgtacctagaacttttcttttgagtgcacactgtacctagtgggccactcgacaaagaaagtatatgcaagtagaaacaagagcgaatccatgagttgacttcacagctaatctcctcctacatgaatttcttcaaataataattaacctcctacattaaagagagagagagagaaacccaaatatagacatcggttgcatacctaattaacccatgaatactttgtctcccgtctactttgccttttctcggttggacacgcgagcgagctaactctgcctgcctgcctgccggccttgatttgcaggtgacaagtcgcagatcaggaagagatacatgcacctaacggaggagatccttcaggagaaccccaacatgtgcgcctacatggcgccctccctggacgcgcgccaggacatcgtcgtcgtggaggtccccaagctcggcaaggcggcggcgcagaaggccatcaaggagtggggacagccgcggtccaagatcacccacctcgtcttctgcaccacctccggcgtcgacatgccgggggccgactaccagctcaccaagatgctcggcctgcgcccgtccgtgaagcgcctcatgatgtaccagcagggctgcttcgccggcggcacggtgctccgcctggccaaggacctcgccgagaacaaccgcggcgcgcgcgtgctcgtcgtctgctcggagatcaccgcggtgaccttccgtggcccgcacgagtcccacctcgactcgctggtgggccaggcgctcttcggcgacggcgcggccgcggtgatcattggcgcggaccccgacgtgtccgtggagcgtcccctgttccagctggtgtccgcgagccagacgatcctgccggactcggagggcgccatcgacggccacctccgggaggtcggcctcaccttccacctcctcaaggacgtgcctgggctcatctccaagaacatcgagcgtgccctagaggaggccttcaagccactgggcatcgacgactggaactccgtcttctggatagcgcacccaggcggcccagccatccttgacatggtggaggccaaggtaaacctcaacaaggagcggatgcgcgcgaccaggcatgtcctctccgagtacggcaacatgtccagtgcctgtgtcctcttcatcatggatgagatgcgcaagcgctccgccgaggatggccacgccacaaccggcgagggaatggactggggcgtcctcttcggcttcggccccggcctcacggtggagaccgtcgtcctccacagcgtccccatcactgccggcgccaccgcgtgatcgctccttctccatttattatcttcctctcatggaaaaacatacaactacagcttctactgccgagtttgtttgtacaatatatattctaccgtgcgtggtttgtatgtgttggtagatgcattctgctgttagtctgtgtcgcgcacgccgtcgcatacttactagtagtaacacaaccgagcttttacaggtttattataagcaaagtgtggtcataatgtggagtgtgaggtgtattacctgtgtacgaaaagattggctgttcaatttgaaccttatgtggttggaaagcaaatggtgattctttcagagctttattgaaatacatattctcctctctcctactttttatttatgaatataatgctttatttaattcataaatgtagcatcaaggaggtacaaaccataacgaacacactcgtctggcctctaaaagaacccagcctcactataggaatcatatatatagtccaacgcatattactcagcaagcacatatagcatggcaacctcataggtatatagaagcaacaagtcacatatataagtatacaacactctccaacaagcatacatatagcatgacaacctcatatagatccatagcataataagaatacaagcaacatagacgctccaagaacaagtttaaaaccaacaagcagcatagacactccaagaaatgcaatgagcttcaccgccgacagcgtccgtgatgaaacctacaagtttagaagcacatgcatgagtttgcaagttcaaatgtcaaacgagctactaggggtaagttcaagctctttatcaaaagatgtgcacacctaagttagaatgtgtcgcttatggcatttgtgggaatgtaatagtggtgtggaagttgtgtgtatggacacaaaatcatataatttatgagaaagcggtgatgacatcaatggttgttgcttatctcattgaaagaggtgacttttcattgcaatcaaatgcaatcaagcatatcagaaactagcctggcctggagtcattgtttcctggagatcgaaggggcatgagttgtaggcgtcactggcttccttgcgcagggccatatgcacctccttgcatattgttcgatccagcactcgga

SEQ ID NO: 16 - DNA, 17050 bp, synthetic construct
  source 1..17050: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcccctaaatagaccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagacgatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagacgaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagacggactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctccgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaacgcgttaactagctaggaattgggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcataatttctactaagtgtagatcgtagttcatcaaagccttcctgtttttttttcctagggggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcataatttctactaagtgtagattcagtgccaggtaggaactcctgttttttttcggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 17 - DNA, 17050 bp, synthetic construct
  source 1..17050: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcccctaaatagaccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagacgatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagacgaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagacggactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctccgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaacgcgttaactagctaggaattgggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcataatttctactaagtgtagatctgaccaagttctgcctcaggagtttttttttcctagggggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcataatttctactaagtgtagatccatcaattaccttatcagtcccttttttttcggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 18 - DNA, 17050 bp, synthetic construct
  source 1..17050: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcccctaaatagaccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagacgatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagacgaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagacggactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctccgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaacgcgttaactagctaggaattgggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcataatttctactaagtgtagatcctgagaagattgaaagccgcattttttttttcctagggggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcataatttctactaagtgtagattccctacctggttcatgagagtcttttttttcggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 19 - DNA, 19 bp, synthetic construct
  source 1..19: mol_type = other DNA
tcagtttacactcatgccg

SEQ ID NO: 20 - DNA, 20 bp, synthetic construct
  source 1..20: mol_type = other DNA
cgaagtcgacagagaacaac

SEQ ID NO: 21 - DNA, 20 bp, synthetic construct
  source 1..20: mol_type = other DNA
ggttttcactcacaccgtcg

SEQ ID NO: 22 - DNA, 20 bp, synthetic construct
  source 1..20: mol_type = other DNA
gttacaaccagatggcaacc

SEQ ID NO: 23 - DNA, 19 bp, synthetic construct
  source 1..19: mol_type = other DNA
tgggagaacaaacctgcgc

SEQ ID NO: 24 - DNA, 20 bp, synthetic construct
  source 1..20: mol_type = other DNA
tttcgaagtcgacggagaac

SEQ ID NO: 25 - DNA, 1785 bp, Zea mays; identical to SEQ ID NO: 4
  source 1..1785: mol_type = genomic DNA

SEQ ID NO: 26 - DNA, 6002 bp, Zea mays
  source 1..6002: mol_type = genomic DNA
ctctcatatgtacctgatattcgaaatgaagatacacaaggaaataaataataaatggtggtctacagtcatatcgtaaattggggtgtgtaacattttatcatgtacgtgctattatttaaatgacaaaatatttacggcgctcgccacttgtaattttggcgaagttatataagtcatgtagtgtgagcgcgcaattaagtcgtgtgacactgtacgtgatctattaacttgtaatttttttttgaagtcataaataataattaatctgaaacagtgaaagttatttatcacaaagtatctgccgctagctcaacaaactagtgagaacacatcactcgtgcgtcgtcgttaatgcgtagcaataggcaatagcaatgtgtcgtacgtattctctaggaaagtaagtagaaactactactagtactactgtgtaggcatgagttaaacacatggaggcatgatttaaacacatgcaatgcattcactgccgcagaaggaaaaaaatgtagagagaggggggggggtgtgattctacaaaaacgaccaatcctaagaatcatcgatcgactgttttacgtcaagcggaataccttttagacgtgcatgcaagtggcatagatcgatgacctacctttcaaaccgtgtttgttttcacatatccaggaaaatataatacgtgtgcgctagagctagctatatatatctagtcctacgtatagttgcgggtaacggttttccggtctttttattaatttaccacggacgacggagacgacgatattgttgaattatttgggcaggctgcagggccgccgacgacggacggagcgtgaatttctgtgcggcacacacggccgtggtagctgaccggacgacgacgagaccggtcgaccgcatgtagtgcaggtgggcgtgcctgatgagggagacgctgactctgactggctgctgctgggtgtgcaagcataccatactctactagtacaccacaccaggcttgccgcccgcccgcccgcggccacgcgacccaactaaccccggcgcggcgttcgttccagtgggcgggtctcgatccccggcacgtccgccccgcgcgtcgtcgccgcccacgtggcggcccatccgcccgtccaactgacctaacccggtcgccagcccgcgcgcttatataaacgcccccaccggcttccgtcctctcccacaccacagctcccacacacagcaggcagcagctcgatcgctagctcctccgaaccggaaagccaagcccgctttggcgatcgtgcttgcttgcttggttccttcaattcctgccggagccagcgagagctagctgctgctgtcgcgtggtagacgtcgtcttgccagcgagagcctagctcgatcggtctctctggtacaacgtagggagaaagatcgagggggagaggacgacgacgatggccggcgcgaccgtgaccgtggaggaggtgaggaaggcccagcgcgccaccggccccgccaccgtgctggcgatcggcaccgccacgcccgccaactgcgtgtaccaggccgactacccggactactacttccggatcaccaagagcgagcacctcaccgacctcaaggagaaattcaagaggatgtgtgagtgcccggcctcgattatatatctagctcactctctctcgccccgtctccatccctcgctcgctcgctagcttgaagaacatacactcacgtcacgtcgtcgtcgtctcctgctacctgcttaattgtttcacgtcgtcggtaggtctcgggcgacgactactagctgacgacggacgatgctagctagctgctgcctcgtgcgcagcagcttccacgtattaattttctccgtcccctcgcgccatgcacaaagacatgaatatttttactaacccacgtagtctattgtctctcctgggtaaacactcgtgggttagttactatctagctactacttatctgaatatttttactagcagtgggagaacagaaagctaatagactacggtcgatcgtacgtgtcgtctcttcgtttaatttttaacagattccaaaaacttctccatcatatgaatatatatactctatgttgcgatccggatagagagagaatataactgcaataaactccttgttgttgagatatgtatatgctgcttgctgctacacaattgctactacctaggcagtcgtacgttacaacaacaacaaacagcagcaaaacttgtgtgtgcaccatgaaacccccgtgttccatgatttttgggggcgcaggtttagcatggacactagctaggctaggctaggatatgttttgggtagggtccatatatatataccagctgctaacgagcgagtaacacacacgtacgcacgcacgcacgcattctagctcggccgataccgtacagacatgaaaaaaaagaaggtgcgtggtcaacgccgcctggccgtgcccgtaccaacaactaacacaaacgttttcattttcttggggtgaaaaaaaaagagagagaagtgagaacgagacgtaaagcaggcgtctgtgcacgggagtgaagagataatacagcttttgtgcccctgtcacgggctgttaggtgtgcaacacaaatggcacgtcgaggcgtcttttgccgccggcctctgctagttcttccatcactgttgcacactgaccactcaagaaacccgctctaattaagctgttgacctcttcttcttcttctagcgcgcgctagctgatgagtagctagcgcaccagagtccagaggtggtagcagtagaaacaggattcttttcgtggacgtcgatgtgccatgctagctgccatcgtccccactgcacggggcagacagggaccccgtgccgtcccatcgactcgactacctcaggcctgcagctagcgcaactcgtatagagagatggaatagtactccatcagcacggcattcccacaccacttggattttgtcgacctgaccggcaagcaactagccccttcctgttccatcaatcgaattgacgtagcaactagctgccttgacgtccgtgctcgggcatggccagtacacatacggccggccggtacggtttgttttaattgggccggtttgattttgacgtgcatgcaggcgacaagtcgatgatccggaagcgttacatgcacctgacggaggagttcctggcggagaacccgagcatgtgcgcgtacatggcgccgtcgctggacgcgcggcaggacgtggtggtggtggaggtgccgaagctggggaaggcggcggcgcagaaggcgatcaaggagtgggggcagccaaagtcgcggatcacgcacctggtgttctgcaccacgtccggggtggacatgccgggcgccgactaccagctgaccaaggcgctgggcctgcgcccctccgtgaaccgcctcatgatgtaccagcaggggtgcttcgcgggcggcacggtgctgcgcgtggccaaggacctcgcggagaacaaccgcggcgcgcgggtgctggtggtgtgctccgagatcacggccgtcacgttccgcggcccctccgagtcgcacctcgactcgctcgtgggccaggcgctgttcggcgacggcgcggcggccgtggtcgtgggcgccgacccggacgaccgcgtcgagcgcccgctcttccagctcgtctccgccgcccagaccatcctgcccgactcggagggcgccatcgacggccacctccgcgaggtggggctcaccttccacctgctcaaggacgtgcctggcctcatctccaagaacatcggccgcgcgctggacgacgcgttcaagccgctcggcatctccgactggaactccatcttctgggtggcgcaccccggcgggcccgccatcctcgaccaggtggaggccaaggtcgggctggacaaggccaggatgcgcgccacccgccacgtcctctccgagtacggcaacatgtccagcgcctgcgtcctcttcatcctcgacgagatgcgcaagcgctccgccgaggacggacaggccaccacgggcgagggcctcgactggggcgtcctcttcggcttcggcccgggactcaccgtcgagaccgtcgtgctccacagcgtccccatcaccaccggagcggccaccgcctgattcatcgattcatcaacgatcaaattcctcccctctccgatccaattcgtcgtcgtctcgttcgtcattattttacgtcgtccgtccgcaaataatgtgctctctgctataattgtcgtgtgtagtagtaagtagctgttactatttttccatgtactgtcagtcgcacaatcatcatatataattaatatctctatatatgcatttcatgcaagaccagggtagagctagcttgaggagaagacccttatcgttgtcactttagcatgcatggggtggtgaggtattgggtatacctgtgtaagacaagcttggctggtttaattatatattttttttaaaaaaaaatctgtttgatacaaaaataacacctgtaatgtgcatggtgacggcaaattgaccgccattttcagtgcacctaaagctgtattggataaatgcttctactcgattattgattgtaagaggtccactgttagcttgtgtacttgtctgatgtgagcccgttgctggtgggcactgtgccatccatctacctgtgcatgaaaagtttttttttttttttttttttgcccaaactactacacacgatgcagaagaaagcgcgattttaaaatatttgatgagtggtcacggtccaggccagcgaggagtttaaacttgagggttcaattggaggtgttgtgtctcccgtgtctggtgtgagaagtggtcctcgatttgccaaggcatcggtagccccggccccccgggatggtaaggtccatcgggccgggttttggctagccagccgggcagagcacaaagctgatgttgatgtgcggatgggggccacggcccagcagcagaaatgataggcatttgtcgttctgacataagtgctatgcatgatagtagtttctttctttttttgctgactgcttattacatgatgtaagtgtaaataatgtcattacaaaaatttgggttctaaaacgacctccgcttctgagccaaacagttttccaaaaaaacctagcttttcttcagcagagcatgcatcagcattttaagaagctataacaaacatacccactgcaccatccatctctcacctcacctttcagacttcactctcacccttgctgctctagtgcacagtctgcagcagactataactttagctcacaattaaattgacttttctatttcagaattaagtaatttacgcagatctgtctcgacacatacgaaaatagtgcacaggggactgaagctgaaacattggagccacggtgcagtggacaagacccaccaaactaggggtttgtccgtttgtgacctattggattgcaacaacagatccgtgaagatgttgaaatatggggaccatcaaactaagctaatgggaagagacatgaatggtacagtttatgttttcacaccgccggcctctgaccttggtcctcgtacgtgtagggatgagaaataattattttatttaattacacttgtaaataatatttttatttaattctggtggcaagaactcagattcagggtctgcatggctgcacctaaaatgagttccttaacaaacaaaaaaaaatatggatgttatacttgttatggtgataagatttctacaatatttgccagtataattttgaaaggaactgaaaagggctcgtaatcgttagaaataccatggttgtaggtaggatacaatgtacctggcatgcatgtgcttttgtgtggaaatgatttcattggtttggaactttgcattcaccagagtaattgactggatcgtacgccaaaaataaaattaagactagctagaaggaacaactcgatt

SEQ ID NO: 27 - DNA, 6000 bp, Zea mays
  source 1..6000: mol_type = genomic DNA
ctctcatatgtacctgatattcgaaatgaagatacacaaggaaataaataataaatggtggtctacagtcatatcgtaaattggggtgtgtaacattttatcatgtacgtgctattatttaaatgacaaaatatttacggcgctcgccacttgtaattttggcgaagttatataagtcatgtagtgtgagcgcgcaattaagtcgtgtgacactgtacgtgatctattaacttgtaatttttttttgaagtcataaataataattaatctgaaacagtgaaagttatttatcacaaagtatctgccgctagctcaacaaactagtgagaacacatcactcgtgcgtcgtcgttaatgcgtagcaataggcaatagcaatgtgtcgtacgtattctctaggaaagtaagtagaaactactactagtactactgtgtaggcatgagttaaacacatggaggcatgatttaaacacatgcaatgcattcactgccgcagaaggaaaaaaatgtagagagaggggggggggtgtgattctacaaaaacgaccaatcctaagaatcatcgatcgactgttttacgtcaagcggaataccttttagacgtgcatgcaagtggcatagatcgatgacctacctttcaaaccgtgtttgttttcacatatccaggaaaatataatacgtgtgcgctagagctagctatatatatctagtcctacgtatagttgcgggtaacggttttccggtctttttattaatttaccacggacgacggagacgacgatattgttgaattatttgggcaggctgcagggccgccgacgacggacggagcgtgaatttctgtgcggcacacacggccgtggtagctgaccggacgacgacgagaccggtcgaccgcatgtagtgcaggtgggcgtgcctgatgagggagacgctgactctgactggctgctgctgggtgtgcaagcataccatactctactagtacaccacaccaggcttgccgcccgcccgcccgcggccacgcgacccaactaaccccggcgcggcgttcgttccagtgggcgggtctcgatccccggcacgtccgccccgcgcgtcgtcgccgcccacgtggcggcccatccgcccgtccaactgacctaacccggtcgccagcccgcgcgcttatataaacgcccccaccggcttccgtcctctcccacaccacagctcccacacacagcaggcagcagctcgatcgctagctcctccgaaccggaaagccaagcccgctttggcgatcgtgcttgcttgcttggttccttcaattcctgccggagccagcgagagctagctgctgctgtcgcgtggtagacgtcgtcttgccagcgagagcctagctcgatcggtctctctggtacaacgtagggagaaagatcgagggggagaggacgacgacgatggccggcgcgaccgtgaccgtggaggaggtgaggaaggcccagcgcgccaccggccccgccaccgtgctggcgatcggcaccgccacgcccgccaactgcgtgtaccaggccgactacccggactactacttccggatcaccaagagcgagcacctcaccgacctcaaggagaaattcaagaggatgtgtgagtgcccggcctcgattatatatctagctcactctctctcgccccgtctccatccctcgctcgctcgctagcttgaagaacatacactcacgtcacgtcgtcgtcgtctcctgctacctgcttaattgtttcacgtcgtcggtaggtctcgggcgacgactactagctgacgacggacgatgctagctagctgctgcctcgtgcgcagcagcttccacgtattaattttctccgtcccctcgcgccatgcacaaagacatgaatatttttactaacccacgtagtctattgtctctcctgggtaaacactcgtgggttagttactatctagctactacttatctgaatatttttactagcagtgggagaacagaaagctaatagactacggtcgatcgtacgtgtcgtctcttcgtttaatttttaacagattccaaaaacttctccatcatatgaatatatatactctatgttgcgatccggatagagagagaatataactgcaataaactccttgttgttgagatatgtatatgctgcttgctgctacacaattgctactacctaggcagtcgtacgttacaacaacaacaaacagcagcaaaacttgtgtgtgcaccatgaaacccccgtgttccatgatttttgggggcgcaggtttagcatggacactagctaggctaggctaggatatgttttgggtagggtccatatatatataccagctgctaacgagcgagtaacacacacgtacgcacgcacgcacgcattctagctcggccgataccgtacagacatgaaaaaaaagaaggtgcgtggtcaacgccgcctggccgtgcccgtaccaacaactaacacaaacgttttcattttcttggggtgaaaaaaaaagagagagaagtgagaacgagacgtaaagcaggcgtctgtgcacgggagtgaagagataatacagcttttgtgcccctgtcacgggctgttaggtgtgcaacacaaatggcacgtcgaggcgtcttttgccgccggcctctgctagttcttccatcactgttgcacactgaccactcaagaaacccgctctaattaagctgttgacctcttcttcttcttctagcgcgcgctagctgatgagtagctagcgcaccagagtccagaggtggtagcagtagaaacaggattcttttcgtggacgtcgatgtgccatgctagctgccatcgtccccactgcacggggcagacagggaccccgtgccgtcccatcgactcgactacctcaggcctgcagctagcgcaactcgtatagagagatggaatagtactccatcagcacggcattcccacaccacttggattttgtcgacctgaccggcaagcaactagccccttcctgttccatcaatcgaattgacgtagcaactagctgccttgacgtccgtgctcgggcatggccagtacatacggccggccggtacggtttgttttaattgggccgggttgattttgacgtgcatgcaggcgacaagtcgatgatccggaagtgttacatgcacctgacggaggagttcctggcggagaacccgagcatgtgcgcgtacatggcgccgtcgctggacgcgcggcaggacgtggtggtggtggaggtgccgaagctggggaaggcggcggcgcagaaggcgatcaaggagtgggggcagccaaagtcgcggatcacgcacctggtgttctgcaccacgtccggggtggacatgccgggcgccgactaccagctgaccaaggcgctgggcctgcgcccctccgtgaaccgcctcatgatgtaccagcaggggtgcttcgcgggcggcacggtgctgcgcgtggccaaggacctcgcggagaacaaccgcggcgcgcgggtgctggtggtgtgctccgagatcacggccgtcacgttccgcggcccctccgagtcgcacctcgactcgctcgtgggccaggcgctgttcggcgacggcgcggcggccgtggtcgtgggcgccgacccggacgaccgcgtcgagcgcccgctcttccagctcgtctccgccgcccagaccatcctgcccgactcggagggcgccatcgacggccacctccgcgaggtggggctcaccttccacctgctcaaggacgtgcctggcctcatctccaagaacatcggccgcgcgctggacgacgcgttcaagccgctcggcatctccgactggaactccatcttctgggtggcgcaccccggcgggcccgccatcctcgaccaggtggaggccaaggtcgggctggacaaggccaggatgcgcgccacccgccacgtcctctccgagtacggcaacatgtccagcgcctgcgtcctcttcatcctcgacgagatgcgcaagcgctccgccgaggacggacaggccaccacgggcgagggcctcgactggggcgtcctcttcggcttcggcccgggactcaccgtcgagaccgtcgtgctccacagcgtccccatcaccaccggagcggccaccgcctgattcatcgattcatcaacgatcaaattcctcccctctccgatccaattcgtcgtcgtctcgttcgtcattattttacgtcgtccgtccgcaaataatgtgctctctgctataattgtcgtgtgtagtagtaagtagctgttactatttttccatgtactgtcagtcgcacaatcatcatatataattaatatctctatatatgcatttcatgcaagaccagggtagagctagcttgaggagaagacccttatcgttgtcactttagcatgcatggggtggtgaggtattgggtatacctgtgtaagacaagcttggctggtttaattatatattttttttaaaaaaaaatctgtttgatacaaaaataacacctgtaatgtgcatggtgacggcaaattgaccgccattttcagtgcacctaaagctgtattggataaatgcttctactcgattattgattgtaagaggtccactgttagcttgtgtacttgtctgatgtgagcccgttgctggtgggcactgtgccatccatctacctgtgcatgaaaagtttttttttttttttttttttgcccaaactactacacacgatgcagaagaaagcgcgattttaaaatatttgatgagtggtcacggtccaggccagcgaggagtttaaacttgagggttcaattggaggtgttgtgtctcccgtgtctggtgtgagaagtggtcctcgatttgccaaggcatcggtagccccggccccccgggatggtaaggtccatcgggccgggttttggctagccagccgggcagagcacaaagctgatgttgatgtgcggatgggggccacggcccagcagcagaaatgataggcatttgtcgttctgacataagtgctatgcatgatagtagtttctttctttttttgctgactgcttattacatgatgtaagtgtaaataatgtcattacaaaaatttgggttctaaaacgacctccgcttctgagccaaacagttttccaaaaaaacctagcttttcttcagcagagcatgcatcagcattttaagaagctataacaaacatacccactgcaccatccatctctcacctcacctttcagacttcactctcacccttgctgctctagtgcacagtctgcagcagactataactttagctcacaattaaattgacttttctatttcagaattaagtaatttacgcagatctgtctcgacacatacgaaaatagtgcacaggggactgaagctgaaacattggagccacggtgcagtggacaagacccaccaaactaggggtttgtccgtttgtgacctattggattgcaacaacagatccgtgaagatgttgaaatatggggaccatcaaactaagctaatgggaagagacatgaatggtacagtttatgttttcacaccgccggcctctgaccttggtcctcgtacgtgtagggatgagaaataattattttatttaattacacttgtaaataatatttttatttaattctggtggcaagaactcagattcagggtctgcatggctgcacctaaaatgagttccttaacaaacaaaaaaaaatatggatgttatacttgttatggtgataagatttctacaatatttgccagtataattttgaaaggaactgaaaagggctcgtaatcgttagaaataccatggttgtaggtaggatacaatgtacctggcatgcatgtgcttttgtgtggaaatgatttcattggtttggaactttgcattcaccagagtaattgactggatcgtacgccaaaaataaaattaagactagctagaaggaacaactcgatt

SEQ ID NO: 28 - DNA, 6001 bp, Zea mays
  source 1..6001: mol_type = genomic DNA
taacaaccatgtgaagacatcctaatttcgatgtaggcagtcgagcaagctggcctcctcttgcggaggaggtaataatagtgcaattgacctttttctaacattattcctgttttcactaaatatgtgagtgtgagtccacgtcgataagatggctccacaatccttaattagcacatggtgaaaaaggtatctgctctaaagtcttattcaaaacctctaaaactaatagtctaaaattaataattagcggctaaaactagccgagaagtttagaacgattcaactaatgaaccagctaactattagctacactttttaaatagtcattagttatcagctgccctaaacccagataaaaccagttaacagtttattagcttgctaacaagtggttctagacgtttggaacggcggcctaatcttttaacaagtggttctagacgtttggaacggcagcctaatctttttttcccctaggtatgtgaacgtacgatgcgagaaggcagacatgggtgttgtggcagcagtagtaagccacattgcgaagcaaaactacttgtgtaaaatcgaagtaaagttagcctcttgttttttgttttaggcgataaccacgttaggcataaagaaccgcagtttcaagtgcaccaactactgaaatccgaatttatgctagtacagctagccatttgatttgatttacttgcgatttgcgagtagcaaagtaagcagatagatgccgttgaggcgttaataccacgctcaccaccacctagtcacccctctacaccggaaggtgttgcctctgccctctgctttccagagttgcactacttgtgccgttgggcttgacgggggcggaagctgtgggctagtccagtgggccaattcccactgatgtgtttgttaagtgcacgcgctgggccggctgctcccttgatagctttttgggcctagttacataccaaccaaggcctctatcttcggccacaccacatgcatgcctgtgatcgaacttgatttatttatatcatcatcattgaaatggagatgcaatgcgctagtattatgtccattcttcctttttagacccagaggtctagagtttgaacaaacggtcagggttttcaacccaccagacaagtacagaaggcggggacggcaatagacctctcactattgaggtgcacgcactggaaaacgacagcctactacaattcctttttagatactactagaagggatttcatttcatacataggataggagtacaaccaacttgagctggtgcactgcaaattaatgacagctcaacaattcatttttttttttgttttgtcttgagcaaacagattaaaatataaaataaaaaaaccagccaagcttgtcttacacaggtactatacaatacctcaccatccatgcggttttatgactccactcttgagctagctcgcccataataaaattaattaagggttttaaaacagctactaggacacaagacagatagtaattaattacgcagatacattattcgtaggtcgggtcgttcatgtctggagcgatcgcgaggatagatggacgactcaggcggcggtgggcgctccggtggtgatggggacgctgtggagcacgacggtctcgacggtgaggcccgggccgaagccgaagaggacgccccagtcgagcccctcgccggtggtggcctggccgtcctcggcggagcgcttgcgcatctcgtccaggatgaagagcacgcaggcgctggacatgttgccgtactcggagaggacgtggcgggtggcgcgcatcctggccttgtccagcccgaccctggcctccacctggtccaggatggcgggcccgccggggtgcgccacccagaagatggagttccagtcggagatgccgagcggctcgaacgcgtcctccagcgcgcgctcgatgttcttggagatgagcccgggcacgtccttgagcaggtggaacgtcagccccacctcgcggaggtggccgtcgatggcgccctccgagtcgggcaggatggtctgcgccgccgacacgagctggaacagcgggcgctcgacccgcccgtccgggtcggcgcccaccacgacggccgccgcgccgtcgccgaacagcgcctgccccacgagcgagtccaggtgcgactcggacggcccgcggaacgtcacggccgtgatctccgagcacaccaccaggaccctcgccccgcggttgttctccgccaggtccttggccacgcgcagcaccgtgccgcccgcgaagcacccctgctggtacatcatcaggcggttcacggacgggcgcagccccagcgccttggtcagctggtagtcggcgcccggcatgtccacccccgacgtggtgcagaacaccaggtgcgtgatccgcgacttgggctgcccccactccttgatcgccttctgcgccgccgccttccccagcttcggcacctccgtcaccaccacgtcctgccgcgcgtccagcgacggcgccatgtacgcgcacatgctcgggttctccgacaggaactcctccgtcaggtgcatgtaccgcttccggatcatcgacttgtcgcctgcacggatgccatggccatgccatgcaatggaattagctaggagcaccgttgccttgacgatggcggatcggccggcatgccgttgctgtctgccctgtgggccctgagcccaacatattcttgggctacaagcatgcaagtacgcgtagtatatagcacgtaggccggcccggcccgtcgccgtccacgaaaagaagcaaataagtgtagcaccagtttgtctcctccggcattaaagtttgattagatttctaagatttgtttgttactagtaccaagagattaacaaggcctgattatttttaattttttttgggaaggttggatcagcagcaggccgtcaactagctaggagtagctagcaactagtagacgcgccttgacttattattattaccatgcttgctactaacagtctgttattattcaacttacacttttttgttattattttcaagatgatccgtacgtgtgctgtcgccggcatgcacgcacggccgttgttgaccctcctccacttcatcacattcccatgaactccgtacggccggtattgggctagctagctagctagctagaatgcacgcttgtcgcgtgctcgtcgatggcccccggaattccaggaacacggggtttttatgcatgtgtggtgcacacaagtttgtgctgttttgtgtcacgtatccagttcatggttatatatcacatatagtctaaggtatctacactagcagaaaagagttcaaagcacgcggtgggatttgattttttgcaaatgaatttgattatcaaccgccagtactatttgtagtggcaatttttttttaaaaaaacagccactagaaaacattttcatccttaatttttttagtttttcaaacgacatcggatggtaaaaccaccaacataaaaatttgtagatcttcaaaagttatggaactttataattgaaaactttttttatttgaaatcatctcgtctctcaaaaaatgcattcgaatttgtcaaatgcaaaatgcaaattttgcaaacgacctcgcatggaaagaaaggtatcaagataaaagttatataacttcaaaagttacgaaactttgtagttaacaacttttttcgtttggattcgttcatagcctcaagtagtcaatttatactcgaatttgttctacgtgggcaacaaaactgcaatatagatacaaggcacgtcatataggtgaagtggtagaggaggcttcgcgtgagggagtggttttttgtgggttagattccaaccggccgcgaattttccgcgaaaaatatcacgacttgcgacttcaacggagccacgaatttgtacaattttttttctactggcccgtagaactagcggatctgaaaaacgctaatgcaaatgtgttcagaaccgactgtatagagcttctctgtaccagtgctgagccttgtatcttatatattaattcactagaacacttaagacatgtctcatttacgtgggttgtatggatgaaatcgtatattagaaccatgctagtgtgctacgtcgtgttttttttcttcacttgtctccttcaaacaccacttaattaagatacgagtaatttatataatataggttgtacgtactgcatgtgccctagctaacaaaccgtttgttggctaattctagtgcgtgcgtggtttaatttgctaccgtacccggggcaatctctctagctaattcttctctacctggacatgtggtcctagctgattaaggattaaatatttggctcgcgtgagtgcatgcatgcatgcatgtctgcttctgtcgtcgtcaacagagtaggaagaagactagtggtgcaataataagtgcggaattttgaagcaagagacgagcgagaccgtagtcatgcatcgctcgatctccactgcagtactgtacggtggtcgtcattgtcaagctatagagtctagtacgacgttataacgccatgcagcatgcgtgcgtggtcgatcgtcgacgagaagaagaaacaagagagcgagcgggagacggtgtgtggacgacgacgatgacgacgacgtgctgaacgaagcaagctcttgcggttttgatgcgtagagcgagcgtggacgacgatagtaggtagggtagggtagggtaggggtagtagagatactcacacatcctcttgaacttctccttgaggtcggtgaggtggtcgctcttggtgatccggaagtagtagtccgggtagtcggcctggtacacgcagttggcgggcgtcgccgtcccgatggccagcacggtggcggggccggtcgcgcgctggcccttcctcacctcgtccacggtcacggtggcgccggccatcgatcgtctctcttcctctcgtacgttgtaccagaggcggccggcaggggtcgtcggagcaagcgagagctagtagtcgtcggaggagctctttcacgctagttagctagtcttaatggcagaggaggcccgccggggtgcggtgcttatatagccgcgcggtcgggttaggtgagttggacgggcgcaagggccgccacggggtgctttagtagttagttgggtcgcgtgggtatggctagttctcgctcgttctcgcgtcgtcacctggtcagctaccacggcttcgtttcaacaataatcgtctctcggtggtgaaaacccacgtactggcaaaccaacctttttccgtagtagtaataatgtagtatatttttacaaccgtacgtcggccctcttgtgttcaggataatggccacccgttacgatttaggaacatgtacgttaacactaaaccagtacgtgttgttgattcatcgatgcactgcaggctagctgcagctgcacgacaatataattgctcgcaggattatggggatccaacaaacaaaagacacgtccacaccacttctacattctgtattaccctcttttttttttgcccttgtctcccttacactactggacacggcttatttgccgagttctgcaaaggctcgaaagcacgcactcgacaaagaaggctcgttaaaacggtggtcatcggtgacaacggcttgtttgtcgggtacttgcaattggccactcggtaaagtaaagtcgtcgtgacggcagcgggtgactgtgacagagccttttgcctgggtctgaggcactcggcaaaaaacctccgacgggcctccctgcatagtttctataccgagtgcgccgtatgacactcgacagaggttgatcctttgccgagtgccagttgctgaagcactcggcaaaagattgcacgtctgccgagtacctgagacatatcactcggcaaacgtggagcctttgccgagtaccttagacataacactcacaaagataatttcccggttaccagc

SEQ ID NO: 29 - DNA, 23 bp, synthetic construct
  source 1..23: mol_type = other DNA
ctgaccaagttctgcctcaggag

SEQ ID NO: 30 - DNA, 23 bp, synthetic construct
  source 1..23: mol_type = other DNA
ccatcaattaccttatcagtccc

SEQ ID NO: 31 - DNA, 23 bp, synthetic construct
  source 1..23: mol_type = other DNA
cgtagttcatcaaagccttcctg

SEQ ID NO: 32 - DNA, 23 bp, synthetic construct
  source 1..23: mol_type = other DNA
tcagtgccaggtaggaactcctg

SEQ ID NO: 33 - DNA, 23 bp, synthetic construct
  source 1..23: mol_type = other DNA
cctgagaagattgaaagccgcat

SEQ ID NO: 34 - DNA, 23 bp, synthetic construct
  source 1..23: mol_type = other DNA
tccctacctggttcatgagagtc

SEQ ID NO: 35 - DNA, 20 bp, synthetic construct
  source 1..20: mol_type = other DNA
ggcaagagctttcctgacca

SEQ ID NO: 36 - DNA, 22 bp, synthetic construct
  source 1..22: mol_type = other DNA
tcttggaggtagctctccacaa

SEQ ID NO: 37 - DNA, 16 bp, synthetic construct
  source 1..16: mol_type = other DNA
ttctgcctcaggagaa

SEQ ID NO: 38 - DNA, 25 bp, synthetic construct
  source 1..25: mol_type = other DNA
ttaattccttcctctgagattttgc

SEQ ID NO: 39 - DNA, 20 bp, synthetic construct
  source 1..20: mol_type = other DNA
ttctccacgcaggagttcct

SEQ ID NO: 40 - DNA, 21 bp, synthetic construct
  source 1..21: mol_type = other DNA
caattaccttatcagtcccag

SEQ ID NO: 41 - DNA, 22 bp, synthetic construct
  source 1..22: mol_type = other DNA
gctgctcacatttccgtagttc

SEQ ID NO: 42 - DNA, 22 bp, synthetic construct
  source 1..22: mol_type = other DNA
caacttcagccagagaagctca

SEQ ID NO: 43 - DNA, 16 bp, synthetic construct
  source 1..16: mol_type = other DNA
aagccttcctgctaat

SEQ ID NO: 44 - DNA, 23 bp, synthetic construct
  source 1..23: mol_type = other DNA
catcgatcactttatcagtgcca

SEQ ID NO: 45 - DNA, 23 bp, synthetic construct
  source 1..23: mol_type = other DNA
gctgaaaatcctttcttggagct

SEQ ID NO: 46 - DNA, 19 bp, synthetic construct
  source 1..19: mol_type = other DNA
taggaactcctgtgtggag

SEQ ID NO: 47 - DNA, 17 bp, synthetic construct
  source 1..17: mol_type = other DNA
gggcgtgatttgcctga

SEQ ID NO: 48 - DNA, 24 bp, synthetic construct
  source 1..24: mol_type = other DNA
cgttgaaatcctttatccctacct

SEQ ID NO: 49 - DNA, 18 bp, synthetic construct
  source 1..18: mol_type = other DNA
agattgaaagccgcatag

SEQ ID NO: 50 - DNA, 18 bp, synthetic construct
  source 1..18: mol_type = other DNA
caggatgcacagcccaaa

SEQ ID NO: 51 - DNA, 22 bp, synthetic construct
  source 1..22: mol_type = other DNA
gaagattgaaagccgcatagaa

SEQ ID NO: 52 - DNA, 17 bp, synthetic construct
  source 1..17: mol_type = other DNA
cctggttcatgagagtc

SEQ ID NO: 53 - DNA, 2199 bp, Oryza sativa
  source 1..2199: mol_type = genomic DNA; note = reverse orientation
  exon 1..983: note = reverse orientation
  intron 984..2002: note = reverse orientation
  exon 2003..2199: note = reverse orientation
tcatgagagtgggttacgcagcaccattgcctctattgtgatacctggtccaaaggctagcattactccccactcaagttgttgatgctgatctccttccttctcccgacggcgacgcaagtcatcaagaacaaacgcgatcgtcgtgccgctcatattcccatactcgttcaacacatggcggcttgccgcaagctttgctggttgcagctgaagctcgccttctatgttgtcaaggatcgcacggccaccagggtgcactgcccagaagaggtcattccaattaggatccatgtttccaaccgatcggaatgtgtcaagcaagcactgccggatgttgtccttgatcagtgttggcacctggatagagaggtggaaatctatgccactggcggtggtctgcatgccaagtgcatgctcagtacttggtatcgttgtttgcgaggcgaggaccatctcgaatagtgggcgctcttcatcgaccagtgggccagcgccgacaatgattgctccggcaccatccccaaacagtccatggccgacaattttgctttcatctgggtttgagaagcatattagcgtcagctcggcacaggctacgagaacgcgtgcatcacggttgttctcggtgagctccttggcaagttgaagtgccctaccgcctccagagcaaccgtggaggctaaggatggtgcgagaaacagaagggttgaggccgagtagtgatgccagcttgaggtcagcgctaggagcactgcacccggagtaggtgctgaagatgaggtgcgtaatgtcaatggccggacggccccactcggcgatggccttccgtgtggctgactcggcgagcttcgggacctcagtagagacgatgtctacacgagtttcaagggagggcatgtgtttgtcgatgatctccgggtgctcacagatgagcttgtcatcaaggtggatgtagcgtttctctattcccgatctgtgacctgaaatggaaaaggatgcacccacatctcttacaattagtacatagcactaagagattataagaataacatgagacatttcaattaagatgccgaatatttacaaaacaagaatatatgatattatgcaagcattttccgtaataaaacaaaaatggtttatatatgtcctatttttaggaagataatcatgagtttaatgttctgaaagattggattagcacatatgtttcgttcaaacatggtctaatgatgtacttgtgggtgcataactctatccactaagactcaattcccggtgccaacatatagacaagtaagtgctatttttcagtacaaatttattaagatccacggatttattgctatgactactctgtagatctcttgtatttttgttaaaagcgtagtctttaaatctatacgagtacagtggcatgggtgcgagatggtagcctgatgagcaggagggacattatatgacaaactatcatatttaacgaggtattagtggtggatccaggatttcagagatggagatgcaaaactttactgttatgattcaagataagagataaagtgaaaaaaatatactagcaaaacacttaaatatagcgagtacaaatacaatgacataaataatgtagaaattcaaatggagcatatagtcctggatgaccttaagtgggcctcgtattgagcatactgcgtatatgtaccaagtatatactgtatatagtagattttttttcaaaaattttgggatgcaaccacaccccatgcatccacattgcatcccacacgcgaaataaagaaaatttggctgaactccaacatcatttcactcttctttctacgtagtatagtataaatatttgtatggtttataatgaattatggcaggagtgtcctaaaaatttaagcgcttgcataatttattctcattagagtttatgaaaaacaactaacgataatgatgatcaatgcaacgatcgaaatgaaaggattgttctacttacatatcctcttcattttgtccttcagctcaataagatgctcgctcttggttagaccaaagtagtagtcggcaaacccatcttgatagacgatatttgccgggtttgcagtgccgatggcgaggaccgcagcagggccttccgcgtgctgtgtgcacaggcggctgtcaacaacagcggcagcagctgctccaggcatg

SEQ ID NO: 54 - DNA, 1828 bp, Oryza sativa
  source 1..1828: mol_type = genomic DNA; note = reverse orientation
  3'UTR 1..134: note = reverse orientation
  exon 135..1156: note = reverse orientation
  intron 1157..1637: note = reverse orientation
  exon 1638..1827: note = reverse orientation
aacactgaaacacacactgatgtagaaattgaagcctgatcgacgtgaacaggaacgatgacacgaaacaattaaaataattaaacaaagtagtgtagtaaagtttacgccagaagttgtaccattaagtagtattactgcataataagaggggcggcggcggtgatggggacgctgtggagaacgatagtctcgacggtgaggccaggaccgaagccgaagaggacgccccagtccatgccctcgccggtggtggcgtggccgtcgtcggcagagagcttgcgcatctcgtcgaggacgaagaggacggtggcgctggccatgttgccgtactcggagaggacgttgcgggaggcgcgcatgcgggccttgtggagggcgaccttctcctcaacctggtcgaggatggccggaccgcccgggtgcaccacccagaagatggagttccagtcgtggacgccgagcggcatgaaggcgtcggtgagcgcgccctcgacactgtcggatatgaactccgggacgtccttgggcaggtggaaggtgagacccacctcgcggaggtgccccacgatggcgtcctccgtgcccggcaggatggtctggctcgccgacacaatctggaacagcggccgctcatcggctgcctcgtcggggtcagagccgacgatcaccgccgctgcgccgtcgccgaagagcgcgtgcccgacgagggagtcgaggtgcgactcggacggcccccggaaggccatcgccatgatctccgagcacaccaccagcacccgcgcgccgcggttgttctcggccaggtccttggccacgcgcagcaccgtgccaccggcgaagcacccctgctggtacatcatcagccgcttcaccgacgtgggtaggcccagcatcttggccacctggtagtcggctccgggcatgtccactccgttggtcgtgcagaacacgaggtgggtgatccgcgacatcggctgcccccactctttgatcgcctcctcggctgccgccttccccagcttgggcacctccgccaccaccatgtcctgcctcgcgtccagcgacggcgccgtgaacacgcacatgctagggttctcctgtaggatctcctcagtaagatgcgtgtacctcttccgtatctgcgagctttcacctgcatatatcatggatgcgcgatattaatttgcacctgcgctattttaatcttaattattattaattattattgttatacggtttagatagggtgaaatttactctggaaattcgcttaaatttttttagaaaatcacgagctgcaattaggagtccgactatcgtctcaagttaatgtgcgagtttttttaagaaatatttcttatacgactccttttgtatttcaaaaaatgaacaaacaataaaattgactcaaacacgaatctatatttccaaaagcgaacgaaattacaaaccgactcaaataaaaatgacataccaaaataccgacaaaaacatgatcagtttttataatagaagtgatggcgcagtactttaatttctataaagggattcacctttcacttatttagactagatacagtatgtgtactagtataatatagatgcaaattaactgattactcattagtgcttacacatcctctggaacctctccttcaggttggttaggtgctcgctgttggtgaccctgaagtagtagtccgggtagtcggtctggtacacgcagttagccggcgttgccgtcccgatcgctagcaccgccgccatcccctccgctctttgcatcctcctaacctcctccaacttcactgttgatgtcaccatc

SEQ ID NO: 55 - DNA, 2138 bp, Oryza sativa
  source 1..2138: mol_type = genomic DNA; note = reverse orientation
  3'UTR 1..343: note = reverse orientation
  exon 344..1338: note = reverse orientation
  intron 1339..1749: note = reverse orientation
  exon 1750..1972: note = reverse orientation
  5'UTR 1973..2138: note = reverse orientation
gctgtgtaaacgaactgtaactgcgcacaaagatctttatttatttaacttttgatattacaaagatcgtcattaagcagtaattaacaaatggcatgcatgaccatgattatagctactccacacttttgggttcaaggtcacttccaaaacacccagttaaggtccaaacatctctccttttagcagagctactccctcaacttgatgccctaattaaaaatgtccatgacccaaacggacaaacaaagtggtaggactcaacacattttattataggacttaccttccaatagaatgcaattcatgtataagtaaaataattaaatttacttaagtttatttaatttcccttgaggccgcttccgacgcggagcaaaatggtctcaacggtgacgcccggcccgaacgccatcagcgcgccccagtcggctccctcggcgccgtcggcgacgaaccggcggcgcagctcgttgagcacgaagatcaccgtggtgccgctcatgttgccgtactcgcgcagcacctgccggctcgccgccatggcgccgttgtccagcccgagcgcctcctcgatgaggtccaggatccttcggccacccgggtggacggcccagaagaggtccctccacctggcggcgacgccgatggcgccgaacgcgtcggccaggcaccgctcgatgctgccggcgagcatctccggcacgccgccgccgatgtggtagtccatgccgcccttcttgtgctgcatggtgatggcgccctcgctgtccgggacggtggcctgcgacgcgaacgccatctcgaacagcgggcgctcgacggcgccgaccgggtcggcgccgacgatgacggcgcccgagccgtcgccgaacagcgcctgcccgatgatggtgtcgttgcaccccacctcgggcccgtagaacgcgatgagcgtggactcggcgcaggcgacgaggacgcgcgcgccgcggttgttctcggcgagctccttggcgagctgcagcgcccggccgccgccgtagcagccgtggaggctgagcacggtgcgcgacacggtggggcggaggccgaggagcgacgccagcaggcggtcgccgccgggcgccttgacgccggagtaggtgctgaagatgaggtgggtgatgtcggtggccggccggccccactcggcgatcgccttcgccgaggcggccgcggcgagctccggcacgaccttggagacgatctccaccctggcgtcgagtgacgtcaggtgtcggtcggcgaactccgggtgtgcctccatgacgtcgtcgttcacgaacatgaaccgcttctcgatgcctgacttgttacctgaaacatacgtacatccacacattagcacacatgaaatgcactaattattgttatttgcttattcaattataatgcgctaaattacactttgtaccggatgaagtattgcaagttaaattttgcatcaaggtgaaaaaaaaataagtttcagttcgcaccatcccaaagatatacaatgagtcaatcgatctaaaatttgttggaattgattgtatagtaaaatggaagttttttttgtgtggaaattgaagcttaattaacactttgcccaatgcaatgtgcatccatatactttttcaatcttgttcttactactacatatatttctaaggtgaaaaagagaagaaaaatggtttgaaattaacaacatatatacacaatgtgcatgcatggatgcataatgcttacagattctcctgagcttgttcttgagctcaggcaggtgctcgctcttggtgacgcggaagtagaagtcggcgtactcatcctgcggcacgcacgtctccgggttcgccgtgccgatggcgagcacggtggccgggccatcggcgcgctgcgcacgccggatctcgtgggtgctgacgacggccgcaggggcggcggcggcggcggtggccggaacaggtgccatgaacgtcgacgctgtgggtgacaaattcaaactatatagcaaatgcaagcgagctagcttagctaggtaagtgcttgtatgatcaatggatcagaggatgctagctatagagatcagcggctgcacaaggagatgggagatcgatgtgtcgtggctgcgtggtttg

SEQ ID NO: 56 - DNA, 3411 bp, Oryza sativa
  source 1..3411: mol_type = genomic DNA; note = reverse orientation
  3'UTR 1..389: note = reverse orientation
  exon 390..1399: note = reverse orientation
  intron 1400..3054: note = reverse orientation
  exon 3055..3241: note = reverse orientation
  5'UTR 3242..3411: note = reverse orientation
aggtacacaaaattaattaagttttcaaattgaatagtaacagccaagcatctcttacacaggtttacaataaacctcaccatccatgacacttaattgacctccctttctcttttcagtatacatatatatgctactaccaaccaaaccacccatacatgacacatgcaaaacaaagtaataattttttttttcactttttatccctaccaacacacggcaaaagggaataactttttaattcgataacgttgttattgtagtagtagttattattaggaaatatatatatatcgatatcgatatatatataatttttttttatgatcgatgataaaagatgacgtgtggcgtacgtaggccggccggcggcgaccagggcggagcgctcaggcggcggcgccggcggtgatggggacgctgtggaggacgacggtctcaacggtgaggccggggccgaagccgaagaggacgccccagtccatgccctcgccggtggtggcgtggccgtcctcggcggagcgcttgcgcatctcgtcgaggatgaagagcacgcaggcgctcgacatgttgccgtactcggacagcacgtggcgcgtcgccctcatcctctccttgtccagcccaaccttcgcctccacctggtccaggatcgccggacctccggggtgcgccacccagaagatggagttccagtccgagatccccagcggtgtgaacgcgtcgcccagcgcgcgctcgatgttcttcgagatgagccccggcacgtccttgagcaggtggaacgtcagcccgacctccctcaggtggccgtcgatggcgccctcgctgtccgggaggatggtctggctcgccgacaccatctggaacagcggccgctcgacggcctcgtcggggtcggagccgacgatcaccgccgccgcgccgtcgccgaacagcgcctgcccgaccatggagtcgaggtgggactcggagggcccccggaacgtcaccgccgtgatctcggagcacacggcgaggacgcgcgcgccgcggttgttctcggcgaggtccttggcgacgcggagcaccgtgccgccggcgaagcacccctgctggtacatcatgaggcggttcacgttgggcctcaggccgagcatcttggcgagctggtagtcggcgccgggcatgtcgacgccggaggtggtgcagaagacgaggtgggtgatgcgggagcgcggctgcccccactccttgatcgccttctgcgccgccgccttccccagcttggggacctcgacgacgacgatgtcctgccgcgcgtccagcgacggcgccatgtacgcgcacatgttggggttctcctgcaggatctcctccgtcaggtgcatgtacctcttcctgatctgcgacttgtcacctgcatccatgacgtcagcagcatccttaatttcccataattaattatttcttctcacagtatccatgttttggcgggaaaatgaaattaattaattaaagaaactcttgtatatatgtatagttaggtgtggaagaattaatcttttttctttttattttcttttttttctcccacaattccatttcctgatcctccgatgacgtcagcatcgtttccctaatttctttttttctcacagtatctattttgggcgggaaaaatgaaattaattaattaagtaactcttgtatatatgtgtagttaagtgtggaagaattaatcagggtaattaactaattagctctccagtcgagtcatgatcgaagtgagagtggccactgctgctgtgtcttgttgctatatggcgtgtgggccctgggtctttataaggggtaggtatagtacgatatcatggacttgtagttgggaaaaaagttttttcttttttttaaggttgaagaaatggttttcgtgcgcaacaaagtaattcagattaattactccttgcaaaaaaagataagaaaaagtgaactcgggtaattaaaaatcgatctatcaatttgcttattttttttatatttagaaaatctatgaacttcaaccgtcaacccataaacgacccgtgaaacgtccaaatccgcctgcttttatgcatgcaaattaatttggtactttaatttgctttctttctttttctccaaaagctgcagtagatcttccatctgatctccttgattaatcaagaagatgcatggtcaactaatcatatattagaaaaattgtaaagcacaggctgaagaaaacatcagcacttcatatctggttgcttggtcaatttttaaagaaatcaagttgcctctatggggtcccttatactcttgacatgtcagtaagtgagacacctgaacctactactcctacacaacctaactagcttgtatgagttagctatatcaggtcaaaccgttgtcctctttccatggccatgcttcttgtgtgttcttgtgtgtgtatggatgaccatgcttaaggataactaacaaactccctaactaattatctcaagacaaaagtactcttagcatgctacaagaagaaacatatatagtattatgtacactgttcccttgtaaacatctgtgcagctatatactagcttgtgattgtccatgtccatggatacacctagctaggtgctagattttgtcatatataactgctagctagtgtatattacttcagagaatgtaagctttgatttttttcctttatagaaaaaaatataatacatatagcttttagctaattataatcccctttttttttcacttctctctgttttttttataactagatggccggcatccatcatgtagctccatacgtacgcatgaggtaattaattaacaaaattaaccaaagagaacaaaccaaagcttgcatatatacatatgcatacagccatcacgtaaaatttcatggagatcatcatatatatatccatgcatgaatgcataatgcatatatataaaaatgcatatggccggattgatcaaaatatattaatgaggtagaagaagaagaagcagagagatgtgagatgggatgcttacacatcctcttgaacttctccttgagctcgaccatgtgctcgctcttggtgatcctgaagtagtagtccgggtagtcggcctggtacacgcagttcgccggcgtcgccgtcccgatcgccagcaccgtcgccggcccctccgccctctgcgccctcctcacctcctccaccgtcaccgccgccgccatctctctcgactaattcaccaggtcaccggcgatgacgaagacgacgatcggtgagcttagctagaaccagcagcagcagcagtgtaagtggcacacacagctagctagctctcgctgatgatgagtagcagcttgctggtgctggggcttgtgcgctgcttgctgctgct

SEQ ID NO: 57 - DNA, 2278 bp, Oryza sativa
  source 1..2278: mol_type = genomic DNA
  5'UTR 1..73
  exon 74..269
  intron 270..1091
  exon 1092..2065
  3'UTR 2066..2278
actgtcacacagcgaaatcacaggactagaatacttgctgactactgaggcgaaaggataccgggtggcgaagatggctgaccttggattcggcgatgccaggagtggcaatggcagcaggagccaatgctccagggggaaggcgatgctgctcgccctcggcaagggcctccctgagcaagttcttccccaggagaaggtcgtcgagacctacctccaggacaccatctgcgacgatcctgcaacaagggcaaagctggaaagactttgtgagttttgttcatgtcacttggtattttacatcctgctttctaggtttgagaattttcagaagccaacatagttggcttagctctcctgaaaaatataactggaccctgagttatcaaaagattgttgtttgttggcttggctgcatcactgtgtgtagaatctagctttatttttatgcatctgagactattaatcatcataggttgataatggaactagtagaatgagaaagataaatagatcatcgtttagtgaaaatgggcacatgatcatcagtactactaaattttagtagcacttctgcttaacaaaaatcagtagggtgtggaaaatgaaattattacagtgaaaaaggcaacagattgtaattagtaaacaacatgtgcacatgataagtactccctaacttacaggagcacttggctctaggaaaatcactacaatgctgcttggcaagtctttggattatagtccaaatatgatattgaattttagatattcattgtatgggttttcttcatcacttttattggtcagctttcttttcatgattgctaaggggacatgtaaaaattcaacctagaattattcttaatcaaagttcaaaatcattttggattatagtctgcagtacaaataagcacaacacaaatttagttgccatttttctggcttggccaagggctgacaaaataagagttataggagataatcacaatatatataccttgaatttgttctgtcatgacttgcacagtccctccaatgcacaaaattgtgggtacttattttcctacaatttgattttttttttacaggcaagaccaccacagtgaggacaaggtacactgtcatgtcaaaggagctcctagacgagcacccagagctcaggactgagggaactccaacactgacgccacggcttgacatctgcaatgctgcagtgcttgagcttggtgctactgcagcccgtgccgcccttggtgaatgggggcgtccagcagctgacatcacccaccttgtctacatctcgtccagtgagcttcgcctcccagggggtgaccttttcctggcaactcgccttggcctccatccaaacaccgtccgcacttcccttctcttccttggctgctccggtggcgctgccgcgctccgcaccgccaaggacattgctgagaacaacccagggagccgcgtccttgtagtagccgcggagacgacggtgctgggattccggccaccaagtcctgaccgtccttacgatcttgttggtgctgccctgtttggtgacggcgcatcagctgcgatcattggagcaggccccattgctgctgaggagagtcccttcctagagcttcagttctcaacacaggagttcctaccagggacggacaaggtaattgatggcaagatcactgaggaagggattaatttcaaactggggcgtgatttgcccgaaaagattgaaaaccgtatagaagggttctgcaggacactcatggatcgggttgggataaaggagttcaatgatgtattctgggctgtgcatcctggtggtccagcaatactgaacaggctagaggtttgccttgaactccagccagagaagctcaagatcagtagaaaggccctgatgaactatggtaatgtgagcagcaacaccgtcttctatgtgttggagtatttgagggatgagttgaagaaagggatgataagggaagaatggggactgatcttggcttttggcccaggcatcacatttgaaggaatgctagttcgaggcattaactgagactgaaaggggtccaagaagactttcagctagatggaagaatcacaaacataccatttccaggttaagtatatagtatgaacttgaaacatctcataaggagcatagatgtcttgacagcatctgtgcactacatagttattgttttactgtaatctatgttttatgtatcaaacaatgtgaggtactctagtttgaatgcaagatacttga